(19) United States
(12) Patent Application Publication
(10) Pub. No.: US 2015/0218141 A1
(43) Pub. Date: Aug. 6, 2015

(54) SUBSTITUTED CARBAMATE COMPOUNDS

(71) Applicant: HOFFMANN-LA ROCHE INC, Little Falls, NJ (US)

(72) Inventors: Christine E. Brotherton-Pleiss, Sunnyvale, CA (US); Zhi Chen, Livingston, NJ (US); Shawn David Erickson, Leonia, NJ (US); Kyungjin Kim, Livingston, NJ (US); Hongju Li, Edison, NJ (US); Allen John Lovey, North Caldwell, NJ (US); Yimin Qian, Wayne, NJ (US); Sung-Sau SO, Verona, NJ (US); Peter Michael Wovkulich, Apalachin, NY (US); Lin Yi, Basking Ridge, NJ (US)

(73) Assignee: HOFFMANN-LA ROCHE INC., Little Falls, NJ (US)

(21) Appl. No.: 14/688,038

(22) Filed: Apr. 16, 2015

Related U.S. Application Data
(63) Continuation of application No. PCT/EP2013/071402, filed on Oct. 14, 2013.
(60) Provisional application No. 61/714,266, filed on Oct. 16, 2012.

(51) Int. Cl. C07D 413/04; C07D 207/09; C07D 223/04; C07D 401/04; C07D 211/34; C07D 401/12

(52) U.S. Cl. CPC C07D 413/04; C07D 211/34; C07D 401/12; C07D 223/04; C07D 401/04; C07D 207/09

(57) ABSTRACT

The invention is concerned with the compounds of formula (I):

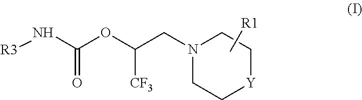

and pharmaceutically acceptable salts thereof, wherein Y, R1 and R3 are defined in the detailed description and claims. In addition, the present invention relates to methods of manufacturing and using the compounds of formula (I) as well as pharmaceutical compositions containing such compounds. The compounds of formula (I) are antagonists of the TRPA1 channel and may be useful in treating inflammatory diseases and disorders associated with that channel.

### CROSS REFERENCE TO RELATED APPLICATIONS

This application is a continuation of International Application No. PCT/EP2013/071402 having an international filing date of Oct. 14, 2013, the entire contents of which are incorporated herein by reference, and which claims benefit under 35 U.S.C. §119 to U.S. Provisional Patent Application No. 61/714,266 filed Oct. 16, 2012, the entire contents of which are incorporated herein by reference.

### FIELD OF THE INVENTION

The present invention relates to organic compounds useful for therapy and/or prophylaxis in a mammal of an inflammatory disease or disorder, and in particular to substituted carbamate compounds, their manufacture, pharmaceutical compositions containing them and their use as Transient Receptor Potential (TRP) channel antagonists.

All documents cited to or relied upon below are expressly incorporated herein by reference.

### BACKGROUND OF THE INVENTION

TRP channels are a class of ion channels found on the plasma membrane of a variety of human (and other animal) cell types. There are at least 28 known human TRP channels which are broken into a number of families or groups based upon sequence homology and function. TRPA1 is a non-selective cation conducting channel that modulates membrane potential via flux of sodium, potassium and calcium. TRPA1 has been shown to be highly expressed in the human dorsal root ganglion neurons and peripheral sensory nerves. In humans, TRPA1 is activated by a number of reactive compounds such as acrolein, allylisothiocyanate, ozone as well as unreactive compounds such as nicotine and menthol and is thus thought to act as a ‘chemosensor’. Many of the known TRPA1 agonists are irritants that cause pain, irritation and neurogenic inflammation in humans and other animals. Therefore, it would be expected that TRPA1 antagonists or agents that block the biological effect of TRPA1 channel activators would be useful in the treatment of diseases such as asthma and its exacerbations, chronic cough and related maladies as well as being useful for the treatment of acute and chronic pain. Recently, it has also been shown that products of tissue damage and oxidative stress, e.g. 4-hydroxynonenal and related compounds, activate the TRPA1 channel. This finding provides additional rationale for the utility of small molecule TRPA1 antagonists in the treatment of diseases related to tissue damage, oxidative stress and bronchial smooth muscle contraction such as asthma, chronic obstructive pulmonary disease (COPD), occupational asthma, and virally-induced lung inflammation.

### SUMMARY OF THE INVENTION

The invention provides for a compound of formula (I):

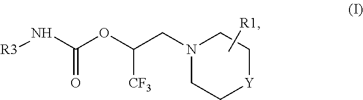

wherein:
Y is —(CH₂)ₙ— or —CF₂—;
n is 0, 1 or 2;
R1 is —X-R2, hydrogen, —CN, —CF₃, alkoxy, cycloalkyl, unsubstituted lower alkyl or lower alkyl substituted with alkoxy;
X is a single bond, —CH₂—, —O—, —C(O)—, S, —CH₂—O— or —O—CH₂—;
R2 is unsubstituted phenyl, phenyl mono- or bi-substituted independently with alkoxy, —CN, —CF₃, —OCF₃, halogen, —O(CH₂)₂OCH₃ or —SO₂CH₃, unsubstituted pyridinyl, pyridinyl substituted with —CN or —CF₃, or methyl-[1,2,4]oxadiaolyl; and
R3 is unsubstituted phenyl, unsubstituted pyridinyl, phenyl mono- or bi-substituted independently with halogen, or pyridinyl mono or bi-substituted independently with halogen, or a pharmaceutically acceptable salt thereof,
with the proviso that said compound is not 1-piperidineethanol-a-(trifluoromethyl)-phenylcarbamate ester, carbamic acid (4-chlorophenyl)-2,2,2-trifluoro-1-(1-piperidinylmethyl)ethyl ester, carbamic acid (3-fluorophenyl)-2,2,2-trifluoro-1-(1-piperidinylmethyl)ethyl ester or carbamic acid (4-methoxyphenyl)-2,2,2-trifluoro-1-(1-piperidinylmethyl)ethyl ester.

The invention also provides for pharmaceutical compositions comprising the compounds, methods of using the compounds and methods of preparing the compounds.

All documents cited to or relied upon below are expressly incorporated herein by reference.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the R variables of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “lower alkyl substituted by halogen” refers to the fact that one or more hydrogen atoms of a lower alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms. In particular embodiments the alkyl has 1 to 10 carbon atoms.

The term “lower alkyl” refers to an alkyl moiety having 1 to 7 carbon atoms. In particular embodiments the lower alkyl has 1 to 4 carbon atoms and in other particular embodiments the lower alkyl has 1 to 3 carbon atoms. Examples of lower alkyls include methyl, ethyl, propyl, isopropyl, butyl, isobutyl, sec-butyl and tert-butyl.

The term “alkoxy” denotes a group of the formula —O—R′, wherein R′ is an alkyl group. Examples of alkoxy moieties include methoxy, ethoxy, isopropoxy, and tert-butoxy.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

The term “heteroaryl” denotes a monovalent aromatic heterocyclic mono- or bicyclic ring system of 5 to 12 ring atoms, comprising 1, 2, 3 or 4 heteroatoms selected from N, O and S, the remaining ring atoms being carbon. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

The terms “halo”, “halogen” and “halide”, which may be used interchangeably, refer to a substituent fluoro, chloro, bromo, or iodo.

“Cycloalkyl” means a monovalent saturated carbocyclic moiety having mono- or bicyclic rings. The cycloalkyl moiety can optionally be substituted with one or more substituents. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

Unless otherwise indicated, the term “hydrogen” or “hydro” refers to the moiety of a hydrogen atom (—H) and not H₂.

In the present description and claims, the representation of hydrogen may be omitted according to the IUPAC convention in the representation of chemical structures. The person skilled in the art therefore understands that when the valence of an atom is not fully represented (e.g. a carbon or nitrogen atom) on a chemical structure, said atom is in fact substituted with one or more hydrogen atoms. For example, “—N—” means “—NH—”.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers”. Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either an individual enantiomer or as a mixture thereof. A mixture containing equal proportions of the enantiomers is called a “racemic mixture”. All such isomers, stereoisomers, enantiomers, chiral compounds and racemic mixtures fall within the scope of the invention described herein.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

In detail, the present invention provides for compounds of formula (I):

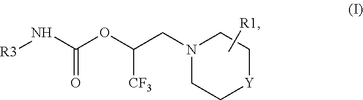

wherein:
Y is —(CH₂)ₙ— or —CF₂—;
n is 0, 1 or 2;
R1 is —X-R2, hydrogen, —CN, —CF₃, alkoxy, cycloalkyl, unsubstituted lower alkyl or lower alkyl substituted with alkoxy;
X is a single bond, —CH₂—, —O—, —C(O)—, S, —CH₂—O— or —O—CH₂—;
R2 is unsubstituted phenyl, phenyl mono- or bi-substituted independently with alkoxy, —CN, —CF₃, —OCF₃, halogen, —O(CH₂)₂OCH₃ or —SO₂CH₃, unsubstituted pyridinyl, pyridinyl substituted with —CN or —CF₃, or methyl-[1,2,4]oxadiaolyl; and
R3 is unsubstituted phenyl, unsubstituted pyridinyl, phenyl mono- or bi-substituted independently with halogen, or pyridinyl mono or bi-substituted independently with halogen,
or a pharmaceutically acceptable salt thereof,
with the proviso that said compound is not 1-piperidineethanol-a-(trifluoromethyl)-phenylcarbamate ester, carbamic acid (4-chlorophenyl)-2,2,2-trifluoro-1-(1-piperidinylmethyl)ethyl ester, carbamic acid (3-fluorophenyl)-2,2,2-trifluoro-1-(1-piperidinylmethyl)ethyl ester or carbamic acid (4-methoxyphenyl)-2,2,2-trifluoro-1-(1-piperidinylmethyl)ethyl ester.

In another embodiment, the invention provides for a compound according to formula (I), wherein Y is —(CH₂)ₙ—.

In another embodiment, the invention provides for a compound according to formula (I), wherein n is 1.

In another embodiment, the invention provides for a compound according to formula (I), wherein R1 is —X-R2.

In another embodiment, the invention provides for a compound according to formula (I), wherein R1 is —CN, —CF₃, alkoxy, cycloalkyl, unsubstituted lower alkyl or lower alkyl substituted with alkoxy.

In another embodiment, the invention provides for a compound according to formula (I), wherein X is a single bond.

In another embodiment, the invention provides for a compound according to formula (I), wherein X is —CH₂—.

In another embodiment, the invention provides for a compound according to formula (I), wherein X is —O—.

In another embodiment, the invention provides for a compound according to formula (I), wherein R2 is unsubstituted phenyl.

In another embodiment, the invention provides for a compound according to formula (I), wherein R2 is phenyl mono- or bi-substituted independently with alkoxy, —CN, —CF₃, —OCF₃, halogen, —O(CH₂)₂OCH₃ or —SO₂CH₃.

In another embodiment, the invention provides for a compound according to formula (I), wherein R2 is unsubstituted pyridinyl.

In another embodiment, the invention provides for a compound according to formula (I), wherein R2 is pyridinyl substituted with —CN or —CF₃.

In another embodiment, the invention provides for a compound according to formula (I), wherein R3 is phenyl mono- or bi-substituted independently with halogen.

In another embodiment, the invention provides for a compound according to formula (I), wherein R3 is pyridinyl mono- or bi-substituted independently with halogen.

In another embodiment, the invention provides for a compound according to formula (I), wherein the compound is:
(4-Chlorophenyl)-carbamic acid 2,2,2-trifluoro-1-[3-(2-methoxyphenyl)-piperidin-1-ylmethyl]-ethyl ester;
(4-Chlorophenyl)-carbamic acid 2,2,2-trifluoro-1-[3-(3-methoxyphenyl)-piperidin-1-ylmethyl]-ethyl ester hydrochloride;
(4-Chlorophenyl)-carbamic acid (S)-2,2,2-trifluoro-1-[(R)-3-(3-methoxyphenyl)-piperidin-1-ylmethyl]-ethyl ester hydrochloride;
(4-Chlorophenyl)-carbamic acid (S)-2,2,2-trifluoro-1-[(S)-3-(3-methoxyphenyl)-piperidin-1-ylmethyl]-ethyl ester hydrochloride;
(4-Chlorophenyl)-carbamic acid (S)-2,2,2-trifluoro-1-((R)-3-phenylpiperidin-1-ylmethyl)-ethyl ester hydrochloride;
(4-Chlorophenyl)-carbamic acid (S)-2,2,2-trifluoro-1-((S)-3-phenylpiperidin-1-ylmethyl)-ethyl ester hydrochloride;
(4-Chlorophenyl)-carbamic acid (R)-2,2,2-trifluoro-1-((R)-3-phenylpiperidin-1-ylmethyl)-ethyl ester hydrochloride;
(4-Chlorophenyl)-carbamic acid (R)-2,2,2-trifluoro-1-((S)-3-phenylpiperidin-1-ylmethyl)-ethyl ester hydrochloride;
(4-Chlorophenyl)-carbamic acid 1-(3-benzylpiperidin-1-ylmethyl)-2,2,2-trifluoroethyl ester hydrochloride salt (1:1);
(4-Chlorophenyl)-carbamic acid (S)-1-((S)-3-benzylpiperidin-1-ylmethyl)-2,2,2-trifluoroethyl ester toluenesulfonic acid salt;
(4-Chlorophenyl)-carbamic acid (S)-1-((R)-3-benzylpiperidin-1-ylmethyl)-2,2,2-trifluoroethyl ester toluenesulfonic acid salt;
(4-Chlorophenyl)-carbamic acid (S)-2,2,2-trifluoro-1-[3-(3-methoxybenzyl)-piperidin-1-ylmethyl]-ethyl ester hydrochloride salt (1:1);
(4-Chlorophenyl)-carbamic acid 1-(3-cyanopiperidin-1-ylmethyl)-2,2,2-trifluoroethyl ester;
(4-Chlorophenyl)-carbamic acid 1-(3-benzyloxypiperidin-1-ylmethyl)-2,2,2-trifluoroethyl ester;
(4-Chlorophenyl)-carbamic acid 2,2,2-trifluoro-1-(3-trifluoromethylpiperidin-1-ylmethyl)-ethyl ester;
(4-Chlorophenyl)-carbamic acid 2,2,2-trifluoro-1-(3-methoxypiperidin-1-ylmethyl)-ethyl ester;
(4-Chlorophenyl)-carbamic acid 1-(3-ethylpiperidin-1-ylmethyl)-2,2,2-trifluoroethyl ester;
(4-Chlorophenyl)-carbamic acid 1-(3-cyclohexylpiperidin-1-ylmethyl)-2,2,2-trifluoroethyl ester;
(4-Chlorophenyl)-carbamic acid 2,2,2-trifluoro-1-[3-(3-methoxypropyl)-piperidin-1-ylmethyl]-ethyl ester;
(4-Chlorophenyl)-carbamic acid 2,2,2-trifluoro-1-[3-(4-methoxybenzyl)-piperidin-1-ylmethyl]-ethyl ester;
(4-Chlorophenyl)-carbamic acid (S)-1-[3-(3,5-dimethoxyphenyl)-piperidin-1-ylmethyl]-2,2,2-trifluoroethyl ester;
(4-Chlorophenyl)-carbamic acid 2,2,2-trifluoro-1-[(S)-3-(pyridin-4-yloxy)-piperidin-1-ylmethyl]-ethyl ester;
(4-Chlorophenyl)-carbamic acid 2,2,2-trifluoro-1-[(R)-3-(pyridin-4-yloxy)-piperidin-1-ylmethyl]-ethyl ester;
(4-Chlorophenyl)-carbamic acid (S)-1-[(R)-3-(3,5-bis-trifluoromethylphenyl)-piperidin-1ylmethyl]-2,2,2-trifluoro-ethyl ester;
(4-Chlorophenyl)-carbamic acid (S)-1-[(S)-3-(3,5-bis-trifluoromethylphenyl)-piperidin-1ylmethyl]-2,2,2-trifluoroethyl ester;
(S)—N-(4-Chlorophenyl)-4,4,4-trifluoro-3-[(R)-3-(3-trifluoromethylphenyl)-piperidin-1-ylmethyl]-butyramide;
(S)—N-(4-Chlorophenyl)-4,4,4-trifluoro-3-[(S)-3-(3-trifluoromethylphenyl)-piperidin-1-ylmethyl]-butyramide;
(4-Chlorophenyl)-carbamic acid 2,2,2-trifluoro-1-(4-phenyl-azepan-1-ylmethyl)-ethyl ester;
(4-Chlorophenyl)-carbamic acid (S)-2,2,2-trifluoro-1-[3-(3-methyl-[1,2,4]oxadiazol-5-yl)-piperidin-1-ylmethyl]-ethyl ester hydrochloride;
(4-Chlorophenyl)-carbamic acid (S)-2,2,2-trifluoro-1-[3-(3-trifluoromethylphenyl)-piperidin-1-ylmethyl]-ethyl ester hydrochloride;
(4-Chlorophenyl)-carbamic acid (S)-2,2,2-trifluoro-1-[(R)-3-(3-trifluoromethylphenyl)-piperidin-1-ylmethyl]-ethyl ester hydrochloride;
(4-Chlorophenyl)-carbamic acid (S)-2,2,2-trifluoro-1-[3-(4-fluoro-3-methoxy-phenyl)-piperidin-1-ylmethyl]-ethyl ester hydrochloride;
(4-Chlorophenyl)-carbamic acid (S)-2,2,2-trifluoro-1-[(R)-3-(4-fluoro-3-methoxyphenyl)-piperidin-1-ylmethyl]-ethyl ester hydrochloride;
(4-Chlorophenyl)-carbamic acid (S)-1-(4,4-difluoropiperidin-1-ylmethyl)-2,2,2-trifluoroethyl ester hydrochloride;
(4-Chlorophenyl)-carbamic acid (S)-2,2,2-trifluoro-1-{3-[4-fluoro-3-(2-methoxyethoxy)-phenyl]-piperidin-1-ylmethyl}-ethyl ester;
(4-Chlorophenyl)-carbamic acid (S)-2,2,2-trifluoro-1-{(R)-3-[4-fluoro-3-(2-methoxy-ethoxy)-phenyl]-piperidin-1-ylmethyl}-ethyl ester hydrochloride;
(4-Chlorophenyl)-carbamic acid 2,2,2-trifluoro-1-(3-phenylpiperidin-1-ylmethyl)-ethyl ester hydrochloride;
(4-Chlorophenyl)-carbamic acid 1-[3-(4-chlorophenyl)-piperidin-1-ylmethyl]-2,2,2-trifluoro ethyl ester;
(4-Chlorophenyl)-carbamic acid (S)-2,2,2-trifluoro-1-[3-(4-methoxyphenyl)-piperidin-1-ylmethyl]-ethyl ester;
(4-Chlorophenyl)-carbamic acid (S)-1-(3-benzoylpiperidin-1-ylmethyl)-2,2,2-trifluoro-ethyl ester hydrochloride;
(4-Chlorophenyl)-carbamic acid (S)-2,2,2-trifluoro-1-(3-phenoxypiperidin-1-ylmethyl)-ethyl ester hydrochloride;
(4-Chlorophenyl)-carbamic acid (S)-2,2,2-trifluoro-1-[3-(3-fluorophenyl)-piperidin-1-ylmethyl]-ethyl ester hydrochloride;
(4-Chlorophenyl)-carbamic acid (S)-2,2,2-trifluoro-1-[3-(4-fluorophenyl)-piperidin-1-ylmethyl]-ethyl ester hydrochloride;
(4-Chlorophenyl)-carbamic acid (S)-1-[3-(3-ethoxyphenyl)-piperidin-1-ylmethyl]-2,2,2-trifluoroethyl ester hydrochloride;
(4-Chlorophenyl)-carbamic acid (S)-2,2,2-trifluoro-1-(3-phenylsulfanylpiperidin-1-ylmethyl)-ethyl ester hydrochloride;
(4-Chlorophenyl)-carbamic acid (S)-1-[3-(4-chlorophenoxy)-piperidin-1-ylmethyl]-2,2,2-trifluoroethyl ester hydrochloride;
(4-Chlorophenyl)-carbamic acid (S)-1-[3-(4-cyanophenoxy)-piperidin-1-ylmethyl]-2,2,2-trifluoroethyl ester hydrochloride;
(4-Chlorophenyl)-carbamic acid (S)-1-[3-(3-cyanophenoxy)-piperidin-1-ylmethyl]-2,2,2-trifluoroethyl ester hydrochloride;
(4-Chlorophenyl)-carbamic acid (S)-1-[3-(4-fluorophenoxy)-piperidin-1-ylmethyl]-2,2,2-trifluoroethyl ester hydrochloride;
(4-Chlorophenyl)-carbamic acid (S)-1-[3-(3-methoxyphenoxy)-piperidin-1-ylmethyl]-2,2,2-trifluoroethyl ester hydrochloride;
(4-Chlorophenyl)-carbamic acid (S)-2,2,2-trifluoro-1-[3-(3-trifluoromethoxyphenoxyl)piperidin-1-ylmethyl]-ethyl ester hydrochloride;
(4-Chlorophenyl)-carbamic acid (S)-2,2,2-trifluoro-1-[3-(4-fluoro-phenoxymethyl)-piperidin-1-ylmethyl]-ethyl ester hydrochloride;
(4-Chlorophenyl)-carbamic acid (S)-1-[(S)-3-(4-chlorophenoxy)-piperidin-1-ylmethyl]-2,2,2-trifluoroethyl ester hydrochloride;
(4-Chlorophenyl)-carbamic acid (S)-1-[(R)-3-(4-chlorophenoxy)-piperidin-1-ylmethyl]-2,2,2-trifluoroethyl ester hydrochloride;
(4-Chlorophenyl)-carbamic acid (S)-2,2,2-trifluoro-1-(3′,4′,5′,6′-tetrahydro-2′H-[2,3]bipyridinyl-1′-ylmethyl)-ethyl ester hydrochloride;
(4-Chlorophenyl)-carbamic acid (S)-1-[3-(4-cyanopyridin-2-yloxy)-piperidin-1-ylmethyl]-2,2,2-trifluoroethyl ester hydrochloride;
(4-Chlorophenyl)-carbamic acid (S)-2,2,2-trifluoro-1-[3-(pyridin-3-ylmethoxy)-piperidin-1-ylmethyl]-ethyl ester hydrochloride;
(4-Chlorophenyl)-carbamic acid (S)-2,2,2-trifluoro-1-(6-trifluoromethyl-3′,4′,5′,6′-tetrahydro-2′H-[2,3]bipyridinyl-1′-ylmethyl)-ethyl ester hydrochloride;
(4-Chlorophenyl)-carbamic acid (S)-2,2,2-trifluoro-1-(6′-trifluoromethyl-3,4,5,6-tetrahydro-2H-[3,3]bipyridinyl-1-ylmethyl)-ethyl ester hydrochloride;
(5-Chloropyridin-2-yl)-carbamic acid (S)-2,2,2-trifluoro-1-[(R)-3-(3-trifluoromethylphenyl)-piperidin-1-ylmethyl]-ethyl ester hydrochloride;
(5-Chloropyridin-2-yl)-carbamic acid (S)-2,2,2-trifluoro-1-[3-(3-trifluoromethylphenyl)-piperidin-1-ylmethyl]-ethyl ester hydrochloride;
(4-Bromophenyl)-carbamic acid (S)-2,2,2-trifluoro-1-[3-(3-methoxyphenyl)-piperidin-1-ylmethyl]-ethyl ester hydrochloride;
(4-Fluorophenyl)-carbamic acid (S)-2,2,2-trifluoro-1-[3-(3-methoxyphenyl)-piperidin-1-ylmethyl]-ethyl ester hydrochloride;
(4-Chlorophenyl)-carbamic acid (S)-2,2,2-trifluoro-1-[3-(3-methanesulfonylphenyl)-piperidin-1-ylmethyl]-ethyl ester hydrochloride;
(4-Fluorophenyl)-carbamic acid (S)-2,2,2-trifluoro-1-[3-(3-methanesulfonylphenyl)-piperidin-1-ylmethyl]-ethyl ester hydrochloride;
(4-Chlorophenyl)-carbamic acid (S)-1-(3-benzylpyrrolidin-1-ylmethyl)-2,2,2-trifluoroethyl ester;
(3-Chloro-4-fluoro-phenyl)-carbamic acid (S)-2,2,2-trifluoro-1-[3-(3-trifluoromethylphenyl)-piperidin-1-ylmethyl]-ethyl ester;
(3,4-Dichlorophenyl)-carbamic acid(S)-2,2,2-trifluoro-1-[3-(3-trifluoromethylphenyl)piperidin-1-ylmethyl]-ethyl ester;
(3,4-Difluorophenyl)-carbamic acid (S)-2,2,2-trifluoro-1-[3-(3-trifluoromethylphenyl)piperidin-1-ylmethyl]-ethyl ester; or
(6-Chloropyridin-3-yl)-carbamic acid (S)-2,2,2-trifluoro-1-[3-(3-trifluoromethylphenyl)-piperidin-1-ylmethyl]-ethyl ester.

Representative compounds of the invention have been shown to modulate TRPA1 activity. Accordingly, the compounds of the invention are useful for treating diseases and conditions mediated by TRPA1 activity. Such diseases and conditions include but are not limited to: pain (acute, chronic, inflammatory, or neuropathic pain); itch or various inflammatory disorders; inner ear disorders; fever or other disorders of thermoregulation; tracheobronchial or diaphragmatic dysfunction; gastrointestinal or urinary tract disorders; chronic obstructive pulmonary disease; incontinence; and disorders associated with reduced blood flow to the CNS or CNS hypoxia.

In a specific embodiment, compounds of the invention can be administered to treat pain, including but not limited to neuropathic and inflammatory pain, among others. Certain types of pain may be considered a disease or disorder, while other types may be considered symptoms of various diseases or disorders, and pain may include various etiologies. Exemplary types of pain treatable with a TRPA1-modulating agent according to the invention include pain associated with, arising from, or caused by: osteoarthritis, rotator cuff disorders, arthritis (e.g., rheumatoid arthritis or inflammatory arthritis; see, Barton et al. Exp. Mol. Pathol. 2006, 81(2), 166-170), fibromyalgia, migraine and headache (e.g. cluster headache, sinus headache, or tension headache; see, Goadsby Curr. Pain Headache Reports 2004, 8, 393), sinusitis, oral mucositis, toothache, dental trauma, dental extractions, dental infections, burn (Bolcskei et al., Pain 2005, 117(3), 368-376), sunburn, dermatitis, psoriasis, eczema, insect sting or bite, musculoskeletal disorders, bony fractures, ligamentous sprains, plantar fasciitis, costochondritis, tendonitis, bursitis, tennis elbow, pitcher's elbow, patellar tendonitis, repetitive strain injury, myofascial syndrome, muscle strain, myositis, temporomandibular joint disorder, amputation, low back pain, spinal cord injury, neck pain, whiplash, bladder spasms, Gl tract disorders, cystitis, interstitial cystitis, cholecystitis, urinary tract infection, urethral colic, renal colic, pharyngitis, cold sores, stomatitis, external otitis, otitis media (Chan et al., Lancet, 2003, 361, 385), burning mouth syndrome, mucositis, esophageal pain, esophageal spasms, abdominal disorders, gastroesophageal reflux disease, pancreatitis, enteritis, irritable bowel disorder, inflammatory bowel disease, Crohn's disease, ulcerative colitis, colon distension, abdominal constriction, diverticulosis, diverticulitis, intestinal gas, hemorrhoids, anal fissures, anorectal disorders, prostatitis, epididymitis, testicular pain, proctitis, rectal pain, labor, childbirth, endometriosis, menstrual cramps, pelvic pain, vulvodynia, vaginitis, orolabial and genital infections (e.g. herpes simplex), pleurisy, pericarditis, non-cardiac chest pain, contusions, abrasions, skin incision (Honore, P. et al., J Pharmacal Exp Ther., 2005, 314, 410-21), postoperative pain, peripheral neuropathy, central neuropathy, diabetic neuropathy, acute herpetic neuralgia, post-herpetic neuralgia, trigeminal neuralgia, glossopharyngeal neuralgia, atypical facial pain, gradiculopathy, HIV associated neuropathy, physical nerve damage, causalgia, reflex sympathetic dystrophy, sciatica, cervical, thoracic or lumbar radiculopathy, brachial plexopathy, lumbar plexopathy, neurodegenerative disorders, occipital neuralgia, intercostal neuralgia, supraorbital neuralgia, inguinal neuralgia, meralgia paresthetica, genitofemoral neuralgia, carpal tunnel syndrome, Morton's neuroma, post-mastectomy syndrome, post-thoracotomy syndrome, post-polio syndrome, Guillain-Barre syndrome, Raynaud's syndrome, coronary artery spasm (Printzmetal's or variant angina), visceral hyperalgesia (Pomonis, J. D. et al. J. Pharmacal. Exp. Ther. 2003, 306, 387; Walker, K. M. et al., J. Pharmacal. Exp. Ther. 2003, 304(1), 56-62), thalamic pain, cancer (e.g. pain caused by cancer, including osteolytic sarcoma, by treatment of cancer by radiation or chemotherapy, or by nerve or bone lesions associated with cancer (see, Menendez, L. et al., Neurosci. Lett. 2005, 393 (1), 70-73; Asai, H. et al., Pain 2005, 117, 19-29), or bone destruction pain (see, Ghilardi, J. R. et al., J. Neurosci. 2005, 25, 3126-31)), infection, or metabolic disease. Additionally, the compounds may be used to treat pain indications such as visceral pain, ocular pain, thermal pain, dental pain, capsaicin-induced pain (as well as other symptomatic conditions induced by capsaicin such as cough, lachrymation, and bronchospasm).

In another specific embodiment, compounds of the invention can be administered to treat itch, which may arise from various sources, such as dermatological or inflammatory disorders.

In another specific embodiment, compounds of the invention can be administered to treat inflammatory disorders, including disorders selected from the group consisting of: renal or hepatobiliary disorders, immunological disorders, medication reactions and unknown/idiopathic conditions. Inflammatory disorders treatable with an inventive agent include, for example, inflammatory bowel disease (lBO), Crohn's disease, and ulcerative colitis (Geppetti, P. et al., Br. J. Pharmacal. 2004, 141, 1313-20; Yiangou, Y. et al., Lancet 2001, 357, 1338-39; Kimball, E. S. et al., Neurogastroenterol. Motif., 2004, 16, 811), osteoarthritis (Szabo, A. et al., J. Pharmacal. Exp. Ther. 2005, 314, 111-119), psoriasis, psoriatic arthritis, rheumatoid arthritis, myasthenia gravis, multiple sclerosis, scleroderma, glomerulonephritis, pancreatitis, inflammatory hepatitis, asthma, chronic obstructive pulmonary disease, allergic rhinitis, uveitis, and cardiovascular manifestations of inflammation including atherosclerosis, myocarditis, pericarditis, and vasculitis.

In another specific embodiment, compounds of the invention can be administered to treat inner ear disorders. Such disorders include, for example, hyperacusis, tinnitus, vestibular hypersensitivity, and episodic vertigo.

In another specific embodiment, compounds of the invention can be administered to treat tracheobronchial and diaphragmatic dysfunctions including, for example, asthma and allergy-related immune responses (Agopyan, N. et al., Am. J. Physiol. Lung Cell Mol. Physiol. 2004, 286, L563-72; Agopyan, N. et al., Toxicol. Appl. Pharmacal. 2003, 192, 21-35), cough (e.g., acute or chronic cough, or cough caused by irritation from gastroesophageal reflux disease; see, Lalloo, U. G. et al., J. Appl. Physiol. 1995, 79(4), 1082-7), bronchospasm, chronic obstructive pulmonary disease, chronic bronchitis, emphysema, and hiccups (hiccoughs, singultus).

In another specific embodiment, compounds of the invention can be administered to treat gastrointestinal and urinary tract disorders such as, bladder overactivity, inflammatory hyperalgesia, visceral hyperreflexia of the urinary bladder, hemorrhagic cystitis (Dinis, P. et al., J Neurosci., 2004, 24, 11253-11263), interstitial cystitis (Sculptoreanu, A. et al., Neurosci Lett., 2005, 381, 42-46), inflammatory prostate disease, prostatitis (Sanchez, M. et al., Eur J Pharmacal., 2005, 515, 20-27), nausea, vomiting, intestinal cramping, intestinal bloating, bladder spasms, urinary urgency, defecation urgency and urge incontinence.

In another specific embodiment, compounds of the invention can be administered to treat disorders associated with reduced blood flow to the CNS or CNS hypoxia. Such disorders include, for example, head trauma, spinal injury, thromboembolic or hemorrhagic stroke, transient ischaemic attacks, cerebral vasospasm, hypoglycaemia, cardiac arrest, status epilepticus, perinatal asphyxia, Alzheimer's disease, and Huntington's Disease.

In other embodiments, compounds of the invention can be administered to treat other diseases, disorders, or conditions mediated through TRPA1 activity, such as: anxiety; learning or memory disorders; eye-related disorders (such as glaucoma, vision loss, increased intraocular pressure, and conjunctivitis); baldness (e.g., by stimulating hair growth); diabetes (including insulin-resistant diabetes or diabetic conditions mediated by insulin sensitivity or secretion); obesity (e.g., through appetite suppression); dyspepsia; biliary colic; renal colic; painful bladder syndrome; inflamed esophagus; upper airway disease; urinary incontinence; acute cystitis; and envenomations (such as marine, snake, or insect stings or bites, including jellyfish, spider, or stingray envenomations).

In one specific embodiment, compounds of the invention are administered to treat pain (including but not limited to acute, chronic, neuropathic and inflammatory pain), arthritis, itch, cough, asthma, or inflammatory bowel disease.

In another embodiment, the invention provides for a method for treating neurpathic pain or inflammatory pain, comprising the step of administering a therapeutically effective amount of a compound according to formula (I) to a subject in need thereof

In another embodiment, the invention provides for a pharmaceutical composition, comprising a therapeutically effective amount of a compound according to formula (I) and a pharmaceutically acceptable carrier.

In another embodiment, the invention provides for a compound according to formula (I) for use as a therapeutically active substance.

In another embodiment, the invention provides for the use of a compound according to formula (I) for the treatment or prophylaxis of a respiratory disorder.

In another embodiment, the invention provides for the use of a compound according to formula (I) for the preparation of a medicament for the treatment or prophylaxis of a respiratory disorder.

In another embodiment, the invention provides for a compound according to formula (I) for the treatment or prophylaxis of a respiratory disorder.

In another embodiment, the invention provides for a method for treating a respiratory disorder selected from chronic obstructive pulmonary disorder (COPD), asthma, allergic rhinitis and bronchospasm, comprising the step of administering a therapeutically effective amount of a compound according to formula (I) to a subject in need thereof

In another embodiment, provided is an invention as hereinbefore described.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Compounds of the invention may be made by any number of conventional means. For example, they may be made according to the processes outlined in Schemes 1 to 4 below.

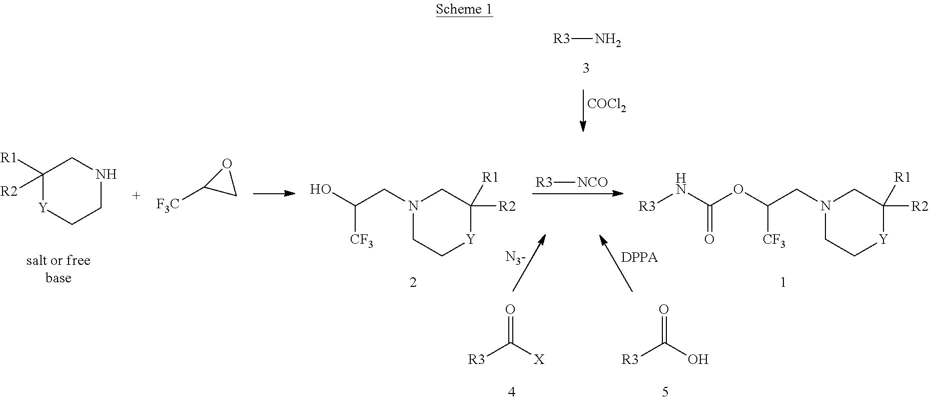

According to Scheme 1, a substituted cyclic amine as a free base or a salt may be reacted with 2-trifluoromethyloxirane to yield a substituted cyclic amino-1,1,1-trifluoropropan-2-ol of formula 2. This transformation is well-documented in the chemical literature and familiar to those skilled in the art. It proceeds under various reactions conditions, for example, the cyclic amine free base and an epoxide can be combined in an aprotic solvent such as dichloromethane or acetonitrile or neat at room temperature or with heating. Alternatively, a common salt of the cyclic amine can be combined in an aprotic solvent such as dichlormethane, tetrahydrofuran or acetonitrile in the presence of a base such as diisoproplyethylamine, triethylamine or cesium carbonate and may be followed by addition of trifluoromethyloxirane. The reaction can proceed at room temperature or with heat. Starting 2-trifluoromethyloxirane is commercially available. A large variety and number of R1,R2-substituted cyclic amines may be purchased from commercial sources or prepared by known procedures. Examples of commercially available cyclic amines include 3-(3-methoxyphenyl)-piperidine, 3-phenylpiperidine, 3-benzylpiperidine, 3-benzylpyrrolidine, 3-(3-methoxybenzyl)piperidine, 3-cyanopiperidine, 3-(benzyloxy)piperidine, 3-(trifluoromethyl)piperidine, 3-methoxypiperidine, 3-ethylpiperidine, 3-cyclohexylmethylpiperidine, 4-(piperidin-3-yloxy)pyridine dihydrochloride, 3-(3-trifluoromethyl-phenyl)-piperidine hydrochloride, 3-(3-methyl-1,2,4-oxadiazol-5-yl)piperidine, 3-(4-chlorophenyl)piperidine, 3-(4-methoxyphenyl)piperidine, 3-(4-fluorophenyl)piperidine hydrochloride, 3-(3-fluorophenyl)piperidine hydrochloride, 3-phenoxypiperidine, 4,4-difluoropiperidine hydrochloride, phenyl(piperidin-3-yl)methanone, 3-(4-fluorophenoxyl)piperidine hydrochloride, 3-(3-methoxyphenoxyl)piperidine, 3-(4-chlorophenoxyl)piperidine, 3-(piperidin-3-yloxy)benzonitrile, 4-(piperidin-3-yloxy)benzonitrile, 3-phenylsulfanylpiperidine hydrochloride, 3-[(3-fluorophenoxy)methyl]piperidine, 2-(piperidin-3-yl)pyridine, 2-(piperidin-3-yl)-6-(trifluoromethyl)pyridine, 3-[(piperidin-3-yloxy)methyl]pyridine, and 3-benzylpyrrolidine. Substituted cyclic amines such as piperidines can alternatively be prepared using published procedures [example: Stamos, D. et al. US2009/0131440, Bagley, S. W. US2002-429506P, U. Hacksell, L.-E. Arvidsson, U. Svensson, and Nilsson, J. Med. Chem. 1981, 12, 1476; K. Kamei et al. Bioorg. and Med. Chem. 2006, 14, 1978; M-Y. Chang, R-T. Hsu, H-P. Chen, and P-J. Lin, Heterocycles 2006, 68, 1173]. Intermediate of formula 2 can then be reacted with isocyanates (R3NCO) by well-established methods to yield compounds of formula 1. For example, the alcohol and the isocyanate (R3NCO) can be combined in an aprotic solvent such as dichloromethane, toluene or acetonitrile at room temperature or with heating. Alternatively the alcohol and the isocyanate (R3NCO) can be combined in an aprotic solvent such as dichloromethane, toluene or acetonitrile followed by the addition of a base such as N,N-diisopropylethylamine or triethylamine at room temperature or with heating. A large variety and number of isocyanates may be purchased from commercial sources or prepared by known procedures. Examples of commercially available isocyanates include 1-isocyanato-4-methyl-benzene, 2-chloro-5-isocyanatopyridine, 1,2-difluoro-4-isocyanatobenzene, 1,2-dichloro-4-isocyanatobenzene, 1-fluoro-4-isocyanatobenzene, 1-bromo-4-isocyanatobenzene, 2-chloro-1-fluoro-4-isocyanatobenzene and 1-chloro-4-isocyanatobenzene. Isocyanates can be prepared using published procedures. The isocyanates may be synthesized from an amine 3 by treatment with phosgene or a phosgene equivalent, such as trichloromethylchloroformate (diphosgene), bis(trichloromethyl)-carbonate (triphosgene), or N,N′-carbonyldiimidazole (CDI). The isocyanate may also be derived from a heterocyclic or aromatic carboxylic acid derivative, such as an ester, an acid halide or an anhydride by a Curtius-type rearrangement. Thus, reaction of acid derivative 4 with an azide source, followed by rearrangement affords the isocyanate. The corresponding carboxylic acid 5 may also be subjected to Curtius-type rearrangements using diphenylphosphoryl azide (DPPA) or a similar reagent.

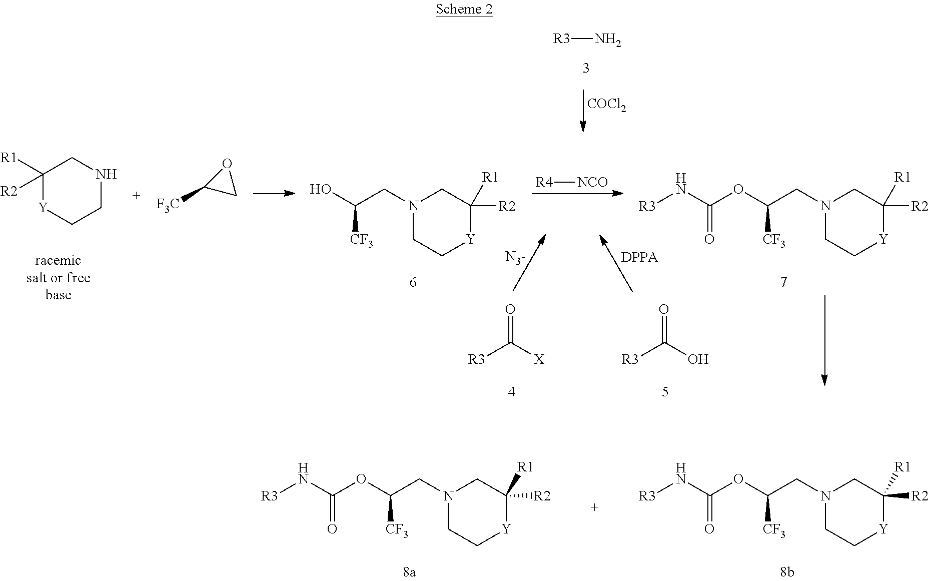

Alternatively, compounds of the invention may be made according to the processes outlined in Scheme 2. A substituted cyclic amine as a free base or a salt may be reacted with a chiral (S) 2-trifluoromethyloxirane to yield a diastereomeric mixture of substituted cyclic amino-1,1,1-trifluoropropan-2-ol of formula 6. This transformation is well-documented in the chemical literature and familiar to those skilled in the art. It proceeds under various reactions conditions, for example, the cyclic amine free base and a chiral epoxide can be combined in an aprotic solvent such as dichloromethane or acetonitrile or neat at room temperature or with heating. Alternatively, a common salt of the cyclic amine can be combined in an aprotic solvent such as dichlormethane, tetrahydrofuran or acetonitrile in the presence of a base such as diisoproplyethylamine, triethylamine or cesium carbonate and may be followed by addition of trifluoromethyloxirane. The reaction can proceed at room temperature or with heat. Starting (S) 2-trifluoromethyloxirane is commercially available. A large variety and number of R1,R2-substituted cyclic amines may be purchased from commercial sources or prepared by known procedures. Examples of commercially available cyclic amines include 3-(3-methoxyphenyl)-piperidine, 3-phenylpiperidine, 3-benzylpiperidine, 3-benzylpyrrolidine, 3-(3-methoxybenzyl)piperidine, 3-cyanopiperidine, 3-(benzyloxy)piperidine, 3-(trifluoromethyl)piperidine, 3-methoxypiperidine, 3-ethylpiperidine, 3-cyclohexylmethylpiperidine, 4-(piperidin-3-yloxy)pyridine dihydrochloride, 3-(3-trifluoromethyl-phenyl)-piperidine hydrochloride, 3-(3-methyl-1,2,4-oxadiazol-5-yl)piperidine, 3-(4-chlorophenyl)piperidine, 3-(4-methoxyphenyl)piperidine, 3-(4-fluorophenyl)piperidine hydrochloride, 3-(3-fluorophenyl)piperidine hydrochloride, 3-phenoxypiperidine, 4,4-difluoropiperidine hydrochloride, phenyl(piperidin-3-yl)methanone, 3-(4-fluorophenoxyl)piperidine hydrochloride, 3-(3-methoxyphenoxyl)piperidine, 3-(4-chlorophenoxyl)piperidine, 3-(piperidin-3-yloxy)benzonitrile, 4-(piperidin-3-yloxy)benzonitrile, 3-phenylsulfanylpiperidine hydrochloride, 3-[(3-fluorophenoxy)methyl]piperidine, 2-(piperidin-3-yl)pyridine, 2-(piperidin-3-yl)-6-(trifluoromethyl)pyridine, 3-[(piperidin-3-yloxy)methyl]pyridine, and 3-benzylpyrrolidine. Substituted cyclic amines such as piperidines can be prepared using published procedures [example: Stamos, D. et al. US2009/0131440 Bagley, S. W. US2002-429506P; U. Hacksell, L.-E. Arvidsson, U. Svensson, and J. L. G. Nilsson, J. Med. Chem. 1981, 12, 1476, K. Kamei et al. Bioorg. and Med. Chem. 2006, 14, 1978; M-Y. Chang, R-T. Hsu, H-P, Chen, and P-J. Lin, Heterocycles 2006, 68, 1173]. Intermediate of formula 6 can then be reacted with isocyanates (R3NCO) by well-established methods to yield compounds of formula 7. For example, the alcohol and the isocyanate (R3NCO) can be combined in an aprotic solvent such as dichloromethane, toluene or acetonitrile at room temperature or with heating. Alternatively the alcohol and the isocyanate (R3NCO) can be combined in an aprotic solvent such as dichloromethane, toluene or acetonitrile followed by the addition of a base such as N-ethyl-N-isopropylpropan-2-amine or triethylamine at room temperature or with heating. A large variety and number of isocyanates may be purchased from commercial sources or prepared by known procedures. Examples of commercially available isocyanates include 1-isocyanato-4-methyl-benzene, 2-chloro-5-isocyanatopyridine, 1,2-difluoro-4-isocyanatobenzene, 1,2-dichloro-4-isocyanatobenzene, 1-fluoro-4-isocyanatobenzene, 1-bromo-4-isocyanatobenzene, 2-chloro-1-fluoro-4-isocyanatobenzene and 1-chloro-4-isocyanatobenzene. Isocyanates can be prepared using published procedures. The isocyanates may be synthesized from an amine 3 by treatment with phosgene or a phosgene equivalent, such as trichloromethylchloroformate (diphosgene), bis(trichloromethyl)-carbonate (triphosgene), or N,N′-carbonyldiimidazole (CDI). The isocyanate may also be derived from a heterocyclic or aromatic carboxylic acid derivative, such as an ester, an acid halide or an anhydride by a Curtius-type rearrangement. Thus, reaction of acid derivative 4 with an azide source, followed by rearrangement affords the isocyanate. The corresponding carboxylic acid 5 may also be subjected to Curtius-type rearrangements using diphenylphosphoryl azide (DPPA) or a similar reagent. The diasteromeric mixture (intermediate 7) can also be separated to yield 8a and 8b by known chromatographic methods of purification such as flash chromatography on silica and/or by reverse-phase preparative HPLC (high performance liquid chromatography) or super critical fluid chromatography. Chromatographic columns can be purchased from commercial sources. Examples of commercially-available columns are SF-15 silica columns, SF-25 silica columns, Prep C₁₈ reverse-phase column, Pirkel's Whelk chiral column and Diacel AD chiral column.

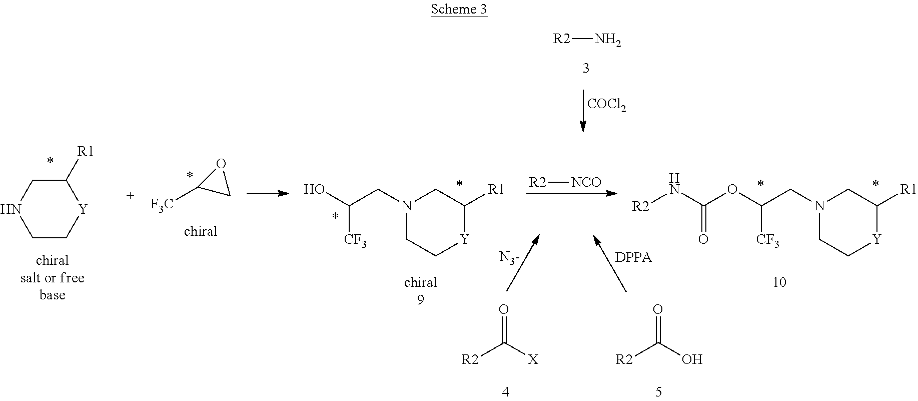

According to Scheme 3, a substituted cyclic amine of (R) configuration as a free base or a salt may be reacted with (S) 2-trifluoromethyloxirane to yield a (S)-1,1,1-trifluoro-3-((R)-3-substituted-piperidin-1-yl)-propan-2-ol or (S)-1,1,1-trifluoro-3-((R)-3-substituted-pyrrolidin-1-yl)-propan-2-ol of formula 9. Alternatively a substituted cyclic amine of (S) configuration as a free base or a salt may be reacted with (S) 2-trifluoromethyloxirane to yield a (S)-1,1,1-trifluoro-3-((S)-3-substituted-piperidin-1-yl)-propan-2-ol or (S)-1,1,1-trifluoro-3-((S)-3-substituted-pyrrolidin-1-yl)-propan-2-ol of formula 9. Alternatively a substituted cyclic amine of (R) configuration as a free base or a salt may be reacted with (R) 2-trifluoromethyloxirane to yield a (R)-1,1,1-trifluoro-3-((R)-3-substituted-piperidin-1-yl)-propan-2-ol or (R)-1,1,1-Trifluoro-3-((R)-3-substituted-pyrrolidin-1-yl)-propan-2-ol of formula 9. Alternatively a substituted cyclic amine of (S) configuration as a free base or a salt may be reacted with (R) 2-trifluoromethyloxirane to yield a (R)-1,1,1-trifluoro-3-((S)-3-substituted-piperidin-1-yl)-propan-2-ol or (R)-1,1,1-trifluoro-3-((S)-3-substituted-pyrrolidin-1-yl)-propan-2-ol of formula 9. This transformation is well-documented in the chemical literature and familiar to those skilled in the art. It proceeds under various reactions conditions, for example, the chiral cyclic amine free base and a chiral epoxide can be combined in an aprotic solvent such as dichloromethane or acetonitrile or neat at room temperature or with heating. Alternatively, a common salt of the chiral cyclic amine can be combined in an aprotic solvent such as dichlormethane, tetrahydrofuran or acetonitrile in the presence of a base such as diisoproplyethylamine, triethylamine or cesium carbonate and may be followed by addition of chiral trifluoromethyloxirane. The reaction can proceed at room temperature or with heat. Starting (S) 2-trifluoromethyloxirane and (R) 2-trifluoromethyloxirane are commercially available. Chiral substituted cyclic amines are commercially available or can be prepared using published procedures or variations thereof [example: M. Amat, M. Canto, N. Llor, C. Escolano, E. Molins, E. Espinosa, and J. Bosch, J. Org. Chem. 2002, 67, 5343; J. J. Verendel, T. Zhou, J-Q. Li, A. Paptchikhine, O. Lebedev, and P. G. Andersson, J. Am. Chem. Soc. 2010, 132, 8880; F. Colpaert, S. Mangelinckx, and N. De Kimpe, J. Org. Chem. 2011, 76, 234 and references cited therein]. Examples of commercially-available chiral cyclic amines are: (S)-3-(3-methoxyphenyl)piperidine, (R)-3-phenyl piperidine, (S)-3-phenylpiperidine, (S)-3-(4-fluorobenzyl)-piperidine hydrochloride, (S)-3-phenylpyrrolidine hydrochloride, (R)-3-phenylpyrrolidine hydrochloride. Intermediate of formula 9 can then be reacted with isocyanates (R2NCO) by well-established methods to yield compounds of formula 10. For example, the alcohol and the isocyanate (R2NCO) can be combined in an aprotic solvent such as dichloromethane, toluene or acetonitrile at room temperature or with heating. Alternatively the alcohol and the isocyanate (R2NCO) can be combined in an aprotic solvent such as dichloromethane, toluene or acetonitrile followed by the addition of a base such as diisopropylethylamine or triethylamine at room temperature or with heating. A large variety and number of isocyanates may be purchased from commercial sources or prepared by known procedures. Examples of commercially available isocyanates include 1-isocyanato-4-methyl-benzene, 2-chloro-5-isocyanatopyridine, 1,2-difluoro-4-isocyanatobenzene, 1,2-dichloro-4-isocyanatobenzene, 1-fluoro-4-isocyanatobenzene, 1-bromo-4-isocyanatobenzene, 2-chloro-1-fluoro-4-isocyanatobenzene and 1-chloro-4-isocyanatobenzene. Isocyanates can be prepared using published procedures. The isocyanates may be synthesized from an amine 3 by treatment with phosgene or a phosgene equivalent, such as trichloromethylchloroformate (diphosgene), bis(trichloromethyl)-carbonate (triphosgene), or N,N′-carbonyldiimidazole (CDI). The isocyanate may also be derived from a heterocyclic or aromatic carboxylic acid derivative, such as an ester, an acid halide or an anhydride by a Curtius-type rearrangement. Thus, reaction of acid derivative 4 with an azide source, followed by rearrangement affords the isocyanate. The corresponding carboxylic acid 5 may also be subjected to Curtius-type rearrangements using diphenylphosphoryl azide (DPPA) or a similar reagent.

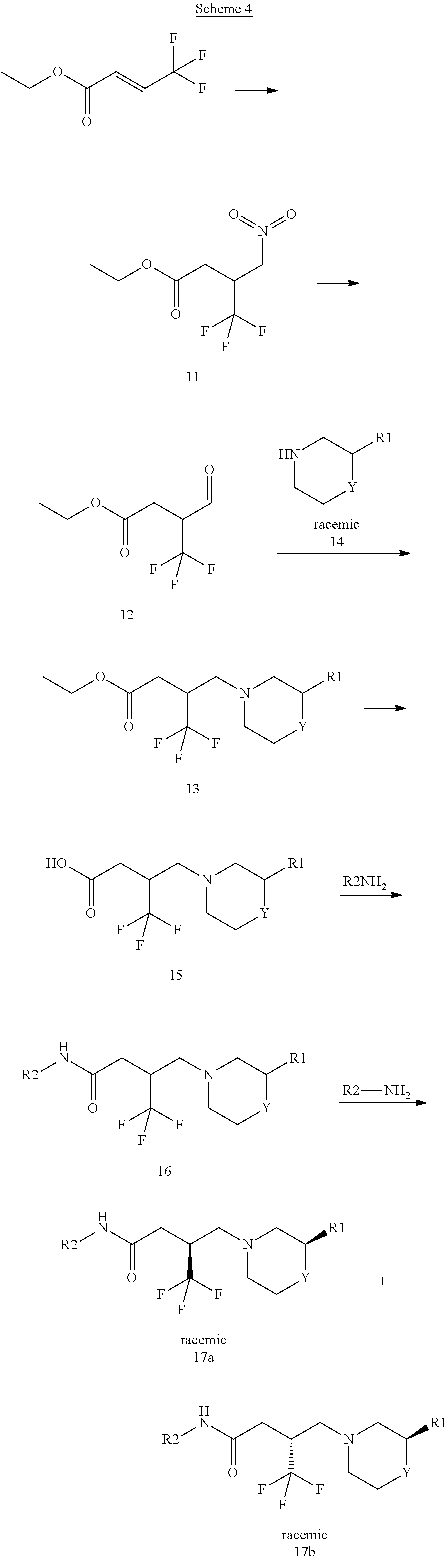

Alternatively, compounds of the invention may be made according to the processes outlined in Scheme 4. Intermediate of formula 11 could be prepared from commercially available (E)-4,4,4-trifluoro-but-2-enoic acid ethyl ester by reaction with nitromethane under Michael reaction conditions. This transformation is well-documented in the chemical literature and familiar to those skilled in the art. It proceeds under various reactions conditions, for example, nitromethane and an α,β-unsaturated ester can be combined in a solvent such as ethanol, ethyl acetate, toluene or acetonitrile or neat at room temperature or with heating in the presence of a base such as DBU, tetramethylguanidine, triethylamine, diisoproplyethylamine or Triton B. Intermediate of formula 11 can then be converted to the aldehyde intermediate 12 by a sequence such as a modified Nef reaction that is described in the literature [for example: Steliou, K. and Poupart, M. A. J. Organic Chem. 1985, 50, 4971]. Intermediate of formula 12 can then be reacted with cyclic amines to yield γ-aminoesters of formula 13. This transformation is well-documented in the chemical literature and familiar to those skilled in the art. It proceeds under various reductive amination reaction conditions, for example, the aldehyde and the cyclic amine 14 can be combined in an aprotic solvent such as dichloroethane, dichloromethane, tetrahydrofuran and treated with a reducing agent such as sodium triacetoxyborohydride. Alternatively the aldehyde and the cyclic amine 14 could be combined in an alcoholic solvent such as ethanol and treated with a reducing agent such as sodium cyanoborohydride or sodium borohydride. A large variety and number of R1,R2-substituted cyclic amines (14) may be purchased from commercial sources or prepared by known procedures. Examples of commercially available cyclic amines include 3-(3-methoxyphenyl)-piperidine, 3-phenylpiperidine, 3-benzylpiperidine, 3-benzylpyrrolidine, 3-(3-methoxybenzyl)piperidine, 3-cyanopiperidine, 3-(benzyloxy)piperidine, 3-(trifluoromethyl)piperidine, 3-methoxypiperidine, 3-ethylpiperidine, 3-cyclohexylmethylpiperidine, 4-(piperidin-3-yloxy)pyridine dihydrochloride, 3-(3-trifluoromethyl-phenyl)-piperidine hydrochloride, 3-(3-methyl-1,2,4-oxadiazol-5-yl)piperidine, 3-(4-chlorophenyl)piperidine, 3-(4-methoxyphenyl)piperidine, 3-(4-fluorophenyl)piperidine hydrochloride, 3-(3-fluorophenyl)piperidine hydrochloride, 3-phenoxypiperidine, 4,4-difluoropiperidine hydrochloride, phenyl(piperidin-3-yl)methanone, 3-(4-fluorophenoxyl)piperidine hydrochloride, 3-(3-methoxyphenoxyl)piperidine, 3-(4-chlorophenoxyl)piperidine, 3-(piperidin-3-yloxy)benzonitrile, 4-(piperidin-3-yloxy)benzonitrile, 3-phenylsulfanylpiperidine hydrochloride, 3-[(3-fluorophenoxy)methyl]piperidine, 2-(piperidin-3-yl)pyridine, 2-(piperidin-3-yl)-6-(trifluoromethyl)pyridine, 3-[(piperidin-3-yloxy)methyl]pyridine, and 3-benzylpyrrolidine. Substituted cyclic amines such as piperidines can be prepared using published procedures [example: Stamos, D. et al. US2009/0131440; Bagley, S. W. US2002-429506P; U. Hacksell, L.-E. Arvidsson, U. Svensson, and J. L. G. Nilsson, J. Med. Chem. 1981, 12, 1476; K. Kamei et al. Bioorg. and Med. Chem. 2006, 14, 1978; M-Y. Chang, R-T. Hsu, H-P, Chen and P-J. Lin, Heterocycles 2006, 68, 1173]. Intermediate of formula 13 can then be reacted under hydrolysis conditions to yield intermediate of formula 15. This transformation is well-documented in the chemical literature and familiar to those skilled in the art. It proceeds under various reactions conditions, for example, the ester is combined in a solvent such as methanol, ethanol, isopropanol, tetrahydrofuran, dioxane and water and treated with a base such as sodium hydroxide or potassium hydroxide with heating or at room temperature.

Intermediate of formula 15 (X═OH) can then be coupled to aromatic amines or heteroaromatic amines (R2-NH₂) by a variety of well-established methods to yield compounds of formula 16. For example, the acid and amine can be combined in a solvent such as dimethylformamide and treated with any number of peptide coupling reagents such as 2-(1H-7-azabenzotriazol-1-yl)-1,1,3,3-tetramethyl uronium hexafluorophosphate methanaminium or bromo-tris-pyrrolidino phosphoniumhexafluorophosphate, dicyclohexyl carbodiimide. Alternatively, intermediate of formula 15 (X═OH) can be converted to the acid chloride of formula 15 (X═Cl) using reagents such as oxalyl chloride or thionyl chloride in a solvent such as dichloromethane with dimethyl formamide and then the intermediate of formula 15 (X═Cl) can converted to the intermediate of formula 16 by reaction with aromatic amines or heteroaromatic amines (R2-NH₂). The mixture (intermediate 16) can be separated to yield racemic 17a and racemic 17b by known chromatographic methods of purification such as flash chromatography on silica and/or by reverse-phase preparative HPLC (high performance liquid chromatography) or super critical fluid chromatography. Chromatographic columns can be purchased from commercial sources. Examples of commercially-available columns are SF-15 silica columns, SF-25 silica columns, Prep C₁₈ reverse-phase column, Pirkel's Whelk chiral column and Diacel AD chiral column. Alternatively, following the same scheme except starting with chiral amine 14 chiral 17a and 17b could be prepared. Chiral substituted cyclic amines are commercially available or can be prepared using published procedures or variations thereof [example: M. Amat, M. Canto, N. Llor, C. Escolano, E. Molins, E. Espinosa, and J. Bosch, J. Org. Chem. 2002, 67, 5343; J. J. Verendel, T. Zhou, J-Q. Li, A. Paptchikhine, O. Lebedev, and P. G. Andersson, J. Am. Chem. Soc. 2010, 132, 8880; F. Colpaert, S. Mangelinckx, and N. De Kimpe, J. Org. Chem. 2011, 76, 234 and references cited therein]. Examples of commercially-available cyclic amines are: (S)-3-(3-methoxyphenyl)piperidine, (R)-3-phenyl piperidine, (S)-3-phenylpiperidine, (S)-3-(4-fluorobenzyl)-piperidine hydrochloride, (S)-3-phenylpyrrolidine hydrochloride, (R)-3-phenylpyrrolidine hydrochloride.

### EXAMPLES

Although certain exemplary embodiments are depicted and described herein, the compounds of the present invention can be prepared using appropriate starting materials according to the methods described generally herein and/or by methods available to one of ordinary skill in the art.

Intermediates and final compounds were purified by either flash chromatography and/or by reverse-phase preparative HPLC (high performance liquid chromatography). Unless otherwise noted, flash chromatography was performed using (1) the Biotage SP1™ system and the Quad 12/25 Cartridge module (from Biotage AB), (2) the ISCO CombiFlash® chromatography instrument (from Teledyne Isco, Inc.), or (3) an Analogix® IntelliFlash280™ chromatography instrument (from Analogix Inc., a subsidiary of Varian Inc.). Unless otherwise noted, the silica gel brand and pore size utilized were: (1) KP-SIL™ 60 Å, particle size: 40-60 micron (from Biotage AB); (2) Silica Gel CAS registry No: 63231-67-4, particle size: 47-60 micron; or (3) ZCX from Qingdao Haiyang Chemical Co., Ltd, pore size: 200-300 mesh or 300-400 mesh. Reverse-phase preparative HPLC was performed using a Waters® Delta-Prep™ 3000 HPLC system from Waters Corporation using one or more of the following columns: a Varian Pursuit® C-18 column (10 μm, 20×150 mm) from Varian, Inc., an Xbridge™ Prep C₁₈ column (5 μm, OBD™ 20×100 mm) from Waters Corporation, or a SunFire™ Prep C₁₈ column (5 μm, OBD™ 30×100 mm) from Waters Corporation.

Mass spectrometry (MS) or high resolution mass spectrometry (HRMS) was performed using a Waters® ZQ™ 4000 (from Waters Corporation), a Waters® Quattro Micro™ API (from Waters Corporation), a Micromass® Platform II (from Micromass, a division of Waters Corporation), a Bruker® Apex®II FTICR with a 4.7 Tesla magnet (from Bruker Corporation), a Waters® Alliance® 2795-ZQ™2000 (from Waters Corporation), or an MDS Sciex™ API-2000™n API (from MDS Inc.). Mass spectra data generally only indicates the parent ions unless otherwise stated. MS or HRMS data is provided for a particular intermediate or compound where indicated.

Nuclear magnetic resonance spectroscopy (NMR) was performed using a Varian® Mercury300 NMR spectrometer (for the ¹H NMR spectra acquired at 300 MHz) and a Varian® Inova400 NMR spectrometer (for the ¹H NMR spectra acquired at 400 MHz) both from Varian Inc. NMR data is provided for a particular intermediate or compound where indicated.

All reactions involving air-sensitive reagents were performed under an inert atmosphere. Reagents were used as received from commercial suppliers unless otherwise noted.

### I. Preparation of Certain Intermediates

### Intermediate A

### 1-Chloro-2-fluoro-4-isocyanatobenzene

4-chloro-3-fluoroaniline (2 g, 13.7 mmol) was dissolved in 60 mL of dichloromethane. Under ice bath, saturated sodium bicarbonate solution (60 mL) was added. The mixture was stirred at 0° C. and triphosgene (1.36 g, 4.58 mmol) was added. The mixture was stirred at 0° C. for 1 hr and then extracted with dichloromethane and water. The organic layer was dried with sodium sulfate and filtered. The filtrate solution was concentrated and the residue was treated with 50 mL of hexanes. The hexane solution was concentrated to remove all solvents. The residue was dissolved in 12 mL of hexanes and filtered. The solution was concentrated and dried to give 1-chloro-2-fluoro-4-isocyanatobenzene as an off white solid (1.69 g). ¹H NMR (400 MHz, CHLOROFORM-d) ppm 6.82-6.90 (m, 1H) 6.93 (dd, J=9.22, 2.65 Hz, 1H) 7.31-7.41 (m, 1H).

### Intermediate B

### (R)-2-(3-Trifluoromethylphenyl)-oxirane

Solutions of 4.006 g (15 mmol) of 2-bromo-1-(3-trifluoromethyl-phenyl)-ethanone, 15 mL of anhydrous tetrahydrofuran and 15 mL of 1M borane-THF in tetrahydrofuran were added simultaneously to a stirring solution of 1.5 mL of 1M (R)-(3aR)-tetrahydro-1-methyl-3,3-diphenyl-1H,3H-pyrrolo[1,2-c][1,3,2]oxazaborole in toluene and 15 mL of anhydrous tetrahydrofuran, cooled in an ice water bath at about 15 degrees, over 12.5 minutes. The cooling bath was removed and the mixture stirred at room temperature. After 1.5 hours, ca 0.48 g of methanol was added dropwise (gas evolution) and the mixture stirred for 5 minutes, then 15 mL of 2 M sodium hydroxide was added over 3 minutes. The mixture was stirred at room temperature. After 1.5 hours, the mixture was concentrated under reduced pressure to remove the tetrahydrofuran, and the remaining aqueous phase was extracted twice with diethyl ether. The combined ether extracts were dried over anhydrous magnesium sulfate, filtered and concentrated under reduced pressure to give 2.553 g (90%) of (R)-2-(3-trifluoromethyl-phenyl)oxirane as a light amber liquid. ¹H NMR (400 MHz, DMSO-d₆) δ ppm 2.87 (dd, J=5.27, 2.51 Hz, 1H) 3.12 (dd, J=5.27, 4.02 Hz, 1H) 4.04 (dd, J=4.27, 2.51 Hz, 1H) 7.54-7.58 (m, 2H) 7.60 (s, 1H) 7.62-7.67 (m, 1H).

### Intermediate C

### 3-[4-Fluoro-3-(2-methoxy-ethoxy)-phenyl]-piperidine hydrochloride

To a stirred solution of 2-fluoro-5-(piperidin-3-yl)phenol hydrochloride (450 mg, 1.94 mmol) in dichloromethane (3 mL) and THF (4 mL) was added triethyl amine (983 mg, 9.71 mmol). After the reaction mixture was stirred for 10 minutes, di-tert-butyl carbonate (372 mg, 2.14 mmol) was added at 0° C. The mixture was stirred for 3 h at room temperature and then concentrated in vacuo. The residue was purified by flash chromatography (20/1 dichloromethane/methanol eluent) to provide 3-(4-fluoro-3-hydroxy-phenyl)-piperidine-1-carboxylic acid tert-butyl ester as a colorless oil (461 mg, 80%), which was used as is in the next step.

A mixture of 3-(4-fluoro-3-hydroxy-phenyl)-piperidine-1-carboxylic acid tert-butyl ester (450 mg, 1.52 mmol), 1-bromo-2-methoxyethane (635 mg, 4.57 mmol), and potassium carbonate (212 mg, 1.52 mmol) in acetone (5 mL) was heated at 60° C. overnight. The reaction mixture was filtered through a celite pad and concentrated in vacuo. The residue was purified by flash chromatography (40/1 dichloromethane/methanol eluent) to provide 3-[4-fluoro-3-(2-methoxy-ethoxy)-phenyl]-piperidine-1-carboxylic acid tert-butyl ester (500 mg, 93%) which was used as is in the next step.

To a solution of 3-[4-fluoro-3-(2-methoxy-ethoxy)-phenyl]-piperidine-1-carboxylic acid tert-butyl ester in THF (4 mL) was added 4M HCl in dioxane. The reaction mixture was stirred overnight and concentrated in vacuo. The residue was triturated with ether and filtered to provide 3-[4-fluoro-3-(2-methoxy-ethoxy)-phenyl]-piperidine hydrochloride. LCMS MH+=254.

### II. Preparation of Certain Embodiments of the Invention

Absolute stereochemistry, where assigned, is based on comparison of biological potency and/or relative retention time on silica gel tlc and chromatography to analogs prepared from chiral building blocks of known absolute configuration, described in examples 3-8.

### Example 1

### (4-Chlorophenyl)-carbamic acid 2,2,2-trifluoro-1-[3-(2-methoxyphenyl)-piperidin-1-ylmethyl]-ethyl ester

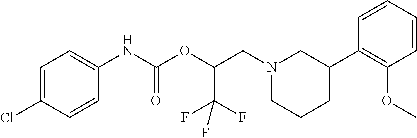 [SMILES: COc1ccccc1C1CCCN(CC(OC(=O)Nc2ccc(Cl)cc2)C(F)(F)F)C1]

### Step 1

To a round bottomed flask containing 0.382 g (2 mmol) of 3-(2-methoxyphenyl)-piperidine was added 0.448 g (4 mmol) of (S)-2-(trifluoromethyl)oxirane dropwise over 10 minutes (exothermic). After 60 minutes, the mixture was transferred to a larger flask, rinsing with acetonitrile, and then volatiles were removed under reduced pressure to give 0.582 g (96%) of 1,1,1-trifluoro-3-[3-(2-methoxy-phenyl)-piperidin-1-yl]-propan-2-ol as an off-white solid.

step 2

In a round bottomed reaction vessel, under argon, was placed 0.303 g (1 mmol) of 1,1,1-trifluoro-3-[3-(2-methoxyphenyl)-piperidin-1-yl]-propan-2-ol, 0.154 g (1 mmol) of 4-chlorophenyl isocyanate, and 0.3 mL of acetonitrile. The vessel was capped, set in a bath at 85 degrees and stirred. After 90 minutes, the mixture was cooled. The crude material was purified by chromatography on silica gel, eluting with hexanes-ethyl acetate (92:8) to give 0.287 g (63%) of (4-chlorophenyl)-carbamic acid 2,2,2-trifluoro-1-[3-(2-methoxyphenyl)-piperidin-1-ylmethyl]-ethyl ester as a white solid. LCMS MH+=457.

### Example 2

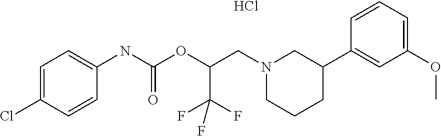 [SMILES: COc1cccc(C2CCCN(CC(OC(=O)Nc3ccc(Cl)cc3)C(F)(F)F)C2)c1 ; Cl]

### (4-Chlorophenyl)-carbamic acid 2,2,2-trifluoro-1-[3-(3-methoxyphenyl)-piperidin-1-ylmethyl]-ethyl ester hydrochloride

In a 15 mL round-bottomed flask, 3-(3-methoxyphenyl)piperidine (268 mg, 1.40 mmol, Eq: 1.00) was combined with acetonitrile (2.88 ml) to give a colorless solution. 2-(trifluoromethyl)oxirane (250 mg, 2.23 mmol, Eq: 1.59) was added and stirred at room temperature for 2 h. The crude reaction mixture was concentrated in vacuo to afford 1,1,1-trifluoro-3-(3-(3-methoxyphenyl)piperidin-1-yl)propan-2-ol as a off-white solid. (400 mg, 94%).

In a 10 mL round-bottomed flask, 1,1,1-trifluoro-3-(3-(3-methoxyphenyl)piperidin-1-yl)propan-2-ol (398 mg, 1.31 mmol, Eq: 1.00) was combined with acetonitrile (2.00 ml) and dichloromethane (0.3 ml) to give a colorless solution. 1-Chloro-4-isocyanatobenzene (202 mg, 1.31 mmol, Eq: 1.00) was added and the reaction mixture was stirred at room temperature for 2 hr. The crude reaction mixture was concentrated in vacuo and diluted with ether/hexane (2 ml/2 ml). A solution of 4N HCl in dioxane (1 ml) was added and the white precipitate formed. The solution was decanted and the solid was washed with hexane and dried to afford (4-chlorophenyl)-carbamic acid 2,2,2-trifluoro-1-[3-(3-methoxyphenyl)-piperidin-1-ylmethyl]-ethyl ester hydrochloride (515 mg, 92%) as an off-white solid. MH+=457.

### Example 3

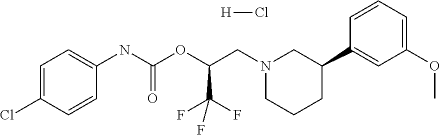

### (4-Chlorophenyl)-carbamic acid (S)-2,2,2-trifluoro-1-[(R)-3-(3-methoxyphenyl)-piperidin-1-ylmethyl]-ethyl ester hydrochloride

(R)-3-(3-methoxyphenyl)-piperidine was obtained by SFC separation of racemic 3-(3-methoxy-phenyl)-piperidine (commercially available from Oakwood). The stereochemistry of (R)-3-(3-methoxyphenyl)-piperidine was established by the comparisons of the SFC chiral analysis of (R)-3-(3-methoxyphenyl)-piperidine with the commercially available (S)-3-(3-methoxyphenyl)-piperidine and with the racemic 3-(3-methoxyphenyl)-piperidine. SFC separation condition (Cellucoat Kromasil OD 3×25 cm, 20% methanol/CO₂ at 70 ml/min, 100 bar, 30° C., 220 nM detection).

In a 15 mL round-bottomed flask, (R)-3-(3-methoxyphenyl)piperidine (160 mg, 837 μmol, Eq: 1.00) was combined with acetonitrile (6 ml) to give a colorless solution. (S)-2-(trifluoromethyl) oxirane (187 mg, 1.67 mmol, Eq: 2) was added and the reaction mixture was stirred at room temperature for 2 h. The crude reaction mixture was concentrated in vacuo and was purified by flash chromatography (silica gel, 30% ethyl acetate in methylene chloride eluent) to afford (S)-1,1,1-trifluoro-3-((R)-3-(3-methoxyphenyl)piperidin-1-yl)propan-2-ol (200 mg, 78%) as an oil. MH+=304.

In a 10 mL round-bottomed flask, (S)-1,1,1-trifluoro-3-((R)-3-(3-methoxyphenyl)piperidin-1-yl)propan-2-ol (200 mg, 659 μmol, Eq: 1.00) was combined with acetonitrile (4.00 ml) to give a colorless solution. 1-chloro-4-isocyanatobenzene (106 mg, 692 μmol, Eq: 1.05) was added and the reaction mixture was stirred at room temperature overnight. The crude reaction mixture was concentrated in vacuo and purified by flash chromatography (silica gel, 20% ethyl acetate in methylene chloride eluent) to give an oil. It was dissolved in ether and hexane and a 4N HCl solution (0.2 ml) was added and mixture concentrated to afford (4-chlorophenyl)-carbamic acid (S)-2,2,2-trifluoro-1-[(R)-3-(3-methoxyphenyl)-piperidin-1-ylmethyl]-ethyl ester hydrochloride (270 mg, 85%) as a white solid. MH+=457.

### Example 4

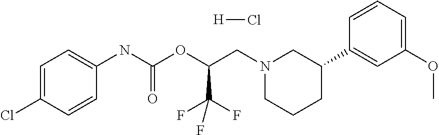

### (4-Chlorophenyl)-carbamic acid (S)-2,2,2-trifluoro-1-[(S)-3-(3-methoxyphenyl)-piperidin-1-ylmethyl]-ethyl ester hydrochloride

In a 15 mL round-bottomed flask, (S)-3-(3-methoxyphenyl)piperidine (88 mg, 460 μmol) was combined with acetonitrile (1 mL) to give a colorless suspension and warmed to make it become a solution. (S)-2-(trifluoromethyl)oxirane (155 mg, 1.38 mmol) was added and the reaction mixture was stirred at room temperature for 2 h. The crude reaction mixture was concentrated in vacuo to afford 135 mg (96%) of (S)-1,1,1-trifluoro-3-[(S)-3-(3-methoxy-phenyl)-piperidin-1-yl]-propan-2-ol as a white solid. LCMS MH+=304.

In a 10 mL round-bottomed flask, (S)-1,1,1-trifluoro-3-((S)-3-(3-methoxyphenyl)piperidin-1-yl)propan-2-ol (135 mg, 445 μmol) was combined with acetonitrile (2.00 ml) to give a colorless solution. 1-chloro-4-isocyanatobenzene (71.8 mg, 467 μmol) was added and the reaction mixture was stirred at room temperature overnight. The crude reaction mixture was concentrated in vacuo and diluted with ether/hexane. A solution of 4N HCl in dioxane (1 ml) was added and a white precipitate formed. The solution was decanted and the precipitate was dried to afford 35 mg (16%) of (4-chlorophenyl)-carbamic acid (S)-2,2,2-trifluoro-1-[(S)-3-(3-methoxyphenyl)-piperidin-1-ylmethyl]-ethyl ester hydrochloride as a white solid. LCMS MH+=457.

### Example 5

(4-Chlorophenyl)-carbamic acid (S)-2,2,2-trifluoro-1-((R)-3-phenylpiperidin-1-ylmethyl)-ethyl ester hydrochloride

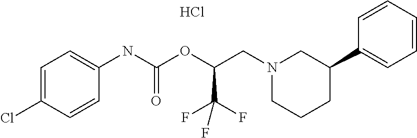 [SMILES: O=C(Nc1ccc(Cl)cc1)O[C@@H](CN1CCC[C@H](c2ccccc2)C1)C(F)(F)F ; Cl]

### Step 1

In a 15 mL round-bottomed flask, (R)-3-phenylpiperidine (100 mg, 0.62 mmol) was combined with acetonitrile (500 μl) to give a colorless suspension and warmed to make it become a solution. (S)-2-(trifluoromethyl)oxirane (208 mg, 1.86 mmol) was added and the reaction mixture was stirred at room temperature for 2 h. The crude reaction mixture was concentrated in vacuo to afford 160 mg (94%) of (S)-1,1,1-trifluoro-3-((R)-3-phenylpiperidin-1-yl)propan-2-ol as a white solid. LCMS MH+=274.

### Step 2

In a 10 mL round-bottomed flask, (S)-1,1,1-trifluoro-3-((R)-3-phenylpiperidin-1-yl)propan-2-ol (160 mg, 585 μmol) was combined with acetonitrile (2 mL) to give a colorless solution. 1-chloro-4-isocyanatobenzene (94.4 mg, 615 μmol) was added and the reaction mixture was stirred at room temperature overnight. The crude reaction mixture was concentrated in vacuo and diluted with ether/hexane. A solution of 4N HCl in dioxane (1 ml) was added and a white precipitate formed. The solution was decanted and the precipitate was dried to afford 112 mg (41%) of (4-chlorophenyl)-carbamic acid (S)-2,2,2-trifluoro-1-((R)-3-phenylpiperidin-1-ylmethyl)-ethyl ester hydrochloride as an off-white solid. LCMS MH+=427.

### Example 6

### (4-Chlorophenyl)-carbamic acid (S)-2,2,2-trifluoro-1-((S)-3-phenylpiperidin-1-ylmethyl)-ethyl ester hydrochloride

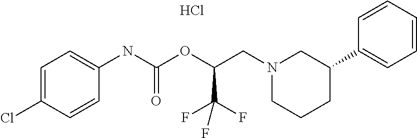 [SMILES: O=C(Nc1ccc(Cl)cc1)O[C@@H](CN1CCC[C@@H](c2ccccc2)C1)C(F)(F)F ; Cl]

Prepared by a similar procedure to example 5 except substituting (R)-3-phenylpiperidine for (S)-3-phenylpiperidine afforded 115 mg of (4-chlorophenyl)-carbamic acid (S)-2,2,2-trifluoro-1-((S)-3-phenylpiperidin-1-ylmethyl)-ethyl ester hydrochloride as an off-white solid. LCMS MH+=427.

### Example 7

### (4-Chlorophenyl)-carbamic acid (R)-2,2,2-trifluoro-1-((R)-3-phenylpiperidin-1-ylmethyl)-ethyl ester hydrochloride

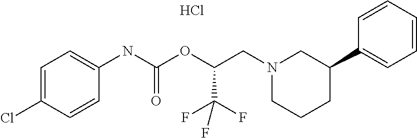 [SMILES: O=C(Nc1ccc(Cl)cc1)O[C@H](CN1CCC[C@H](c2ccccc2)C1)C(F)(F)F ; Cl]

Prepared by a similar procedure to example 5 except substituting (R)-2-(trifluoromethyl)oxirane for (S)-2-(trifluoromethyl)oxirane afforded 150 mg of (4-chlorophenyl)-carbamic acid (R)-2,2,2-trifluoro-1-((R)-3-phenylpiperidin-1-ylmethyl)-ethyl ester hydrochloride as an off-white solid. LCMS MH+=427.

### Example 8

### (4-Chlorophenyl)-carbamic acid (R)-2,2,2-trifluoro-1-((S)-3-phenylpiperidin-1-ylmethyl)-ethyl ester hydrochloride

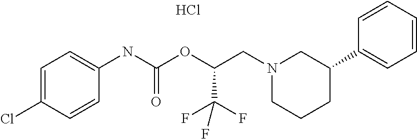 [SMILES: O=C(Nc1ccc(Cl)cc1)O[C@H](CN1CCC[C@@H](c2ccccc2)C1)C(F)(F)F ; Cl]

Prepared by a similar procedure to example 6 except substituting (R)-2-(trifluoromethyl)oxirane for (S)-2-(trifluoromethyl)oxirane afforded 85 mg of (4-chlorophenyl)-carbamic acid (R)-2,2,2-trifluoro-1-((S)-3-phenylpiperidin-1-ylmethyl)-ethyl ester hydrochloride as an off-white solid. LCMS MH+=427.

### Example 9

### (4-Chlorophenyl)-carbamic acid 1-(3-benzylpiperidin-1-ylmethyl)-2,2,2-trifluoroethyl ester hydrochloride salt (1:1)

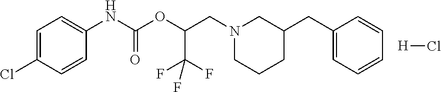

Prepared by a similar procedure to example 1 except substituting 3-benzyl piperidine for 3-(2-methoxyphenyl)-piperidine afforded 0.346 g (78%) of (4-chlorophenyl)-carbamic acid 1-(3-benzylpiperidin-1-ylmethyl)-2,2,2-trifluoroethyl ester as a clear oil.

This material was dissolved in 1 mL of dioxane, treated with 1 mL of a solution of 4 M hydrogen chloride in dioxane. Volatiles were removed under reduced pressure to give (4-chlorophenyl)-carbamic acid 1-(3-benzylpiperidin-1-ylmethyl)-2,2,2-trifluoro-ethyl ester hydrochloride salt (1:1) as a white foam. LCMS MH+=441.

### Example 10

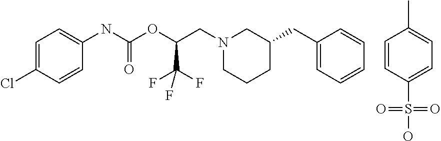

### (4-Chlorophenyl)-carbamic acid (S)-1-((S)-3-benzylpiperidin-1-ylmethyl)-2,2,2-trifluoro-ethyl ester toluenesulfonic acid salt

### Step 1

To a round bottomed flask containing 0.342 g (1.95 mmol) of 3-benzyl piperidine was added 0.219 g (8.9 mmol) of (S)-2-(trifluoromethyl)oxirane (exothermic). After 90 minutes, volatiles were removed under reduced pressure to give (S)-3-(3-benzylpiperidin-1-yl)-1,1,1-trifluoropropan-2-ol as an oil. Intermediate was carried on as is to the next step.

### Step 2

To the (S)-3-(3-benzylpiperidin-1-yl)-1,1,1-trifluoropropan-2-ol from step 1, was added 0.30 g (1.95 mmol) of 4-chlorophenyl isocyanate and ca 0.25 mL of acetonitrile. The mixture was heated at 85 degrees for 90 minutes, cooled, and volatiles removed under reduced pressure. The crude material was purified by chromatography on silica gel, eluting with hexanes-ethyl acetate (90:10) to give 0.717 g (83%) of a mixture of (4-chlorophenyl)-carbamic acid (S)-1-((S)-3-benzylpiperidin-1-ylmethyl)-2,2,2-trifluoro-ethyl ester and (4-chlorophenyl)-carbamic acid (S)-1-((R)-3-benzylpiperidin-1-ylmethyl)-2,2,2-trifluoro-ethyl ester. Diastereomer separation was done by super critical fluid chromatography, (solvent modifier was methanol-ethanol-isopropanol 1:1:1) to give 0.294 g (34%) of (4-chlorophenyl)-carbamic acid (S)-1-((S)-3-benzylpiperidin-1-ylmethyl)-2,2,2-trifluoro-ethyl ester as the first eluting peak and 0.318 g (37%) of (4-chloro-phenyl)-carbamic acid (S)-1-((R)-3-benzyl-piperidin-1-ylmethyl)-2,2,2-trifluoro-ethyl ester as the later eluting peak.

### Step 3

A solution of 0.102 g of toluenesulfonic acid monohydrate in 1 mL of methanol was added to a solution of (4-chlorophenyl)-carbamic acid (S)-1-((S)-3-benzylpiperidin-1-ylmethyl)-2,2,2-trifluoroethyl ester (0.294 g) in 1 mL of methanol. Volatiles were removed under reduced pressure, and the residue was crystallized from acetonitrile to give 0.122 g of (4-chlorophenyl)-carbamic acid (S)-1-((S)-3-benzylpiperidin-1-ylmethyl)-2,2,2-trifluoro-ethyl ester toluenesulfonic acid salt (1:1) as a white solid. LCMS MH+=441.

### Example 11

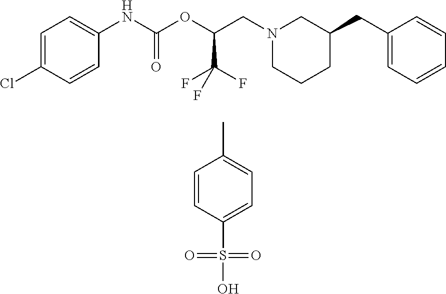

### (4-Chlorophenyl)-carbamic acid (S)-1-((R)-3-benzylpiperidin-1-ylmethyl)-2,2,2-trifluoroethyl ester toluenesulfonic acid salt

(4-chlorophenyl)-carbamic acid (S)-1-((R)-3-benzylpiperidin-1-ylmethyl)-2,2,2-trifluoroethyl ester was prepared as described in example 10 (steps 1-2).

A solution of 0.110 g of toluenesulfonic acid monohydrate in 1 mL of methanol was added to a solution of 0.267 g of (4-chlorophenyl)-carbamic acid (S)-1-((R)-3-benzylpiperidin-1-ylmethyl)-2,2,2-trifluoroethyl ester (0.2674 g) in 1 mL of methanol. Volatiles were removed under reduced pressure to give 0.381 g of (S)-1-((R)-3-benzylpiperidin-1-ylmethyl)-2,2,2-trifluoroethyl ester toluenesulfonic acid salt (1:1) as a white solid. LCMS MH+=441.

### Example 12

(4-Chlorophenyl)-carbamic acid (S)-2,2,2-trifluoro-1-[3-(3-methoxybenzyl)-piperidin-1-ylmethyl]-ethyl ester hydrochloride salt (1:1)

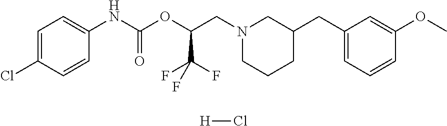 [SMILES: COc1cccc(CC2CCCN(C[C@H](OC(=O)Nc3ccc(Cl)cc3)C(F)(F)F)C2)c1 ; Cl]

To a round bottomed flask containing 0.437 g (2.1 mmol) of 3-(3-methoxybenzyl)-piperidine was added 0.239 g (2.1 mmol) of (S)-2-(trifluoromethyl)oxirane (exothermic). After 90 minutes, volatiles were removed under reduced pressure to give (S)-1,1,1-trifluoro-3-[3-(3-methoxybenzyl)-piperidin-1-yl]-propan-2-ol as a light amber oil.

To the (S)-1,1,1-trifluoro-3-[3-(3-methoxybenzyl)-piperidin-1-yl]-propan-2-ol, prepared above, was added 0.327 g (2.1 mmol) of 4-chlorophenyl isocyanate and ca 0.3 mL of acetonitrile. The mixture was heated at 85 degrees for 90 minutes, cooled, and volatiles removed under reduced pressure. The crude material was purified by chromatography on silica gel, eluting with hexanes-ethyl acetate (90:10) to give 0.570 g (57%) of a (4-chlorophenyl)-carbamic acid (S)-2,2,2-trifluoro-1-[3-(3-methoxybenzyl)-piperidin-1-ylmethyl]-ethyl ester. This material was dissolved in 4 mL dioxane and ca 0.5 mL of 4M HCl in dioxane was added. Volatiles were removed under reduced pressure, then under vacuum, 0.05 mm Hg, to give a 0.6613 g of (4-chlorophenyl)-carbamic acid (S)-2,2,2-trifluoro-1-[3-(3-methoxybenzyl)-piperidin-1-ylmethyl]-ethyl ester hydrochloride salt (1:1) as a white foam. LCMS MH+=471.

### Example 13

### (4-Chlorophenyl)-carbamic acid 1-(3-cyanopiperidin-1-ylmethyl)-2,2,2-trifluoroethyl ester

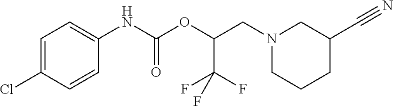 [SMILES: N#CC1CCCN(CC(OC(=O)Nc2ccc(Cl)cc2)C(F)(F)F)C1]

A mixture of 0.200 g (1.8 mmol) of piperidine-3-carbonitrile, 0.306 g (2.7 mmol) of (S)-2-(trifluoromethyl)oxirane and 5 mL of dichloromethane was stirred for 16 hours at room temperature and then concentrated under reduced pressure. The crude material was purified by chromatography on silica gel, eluting with dichloromethane-methanol (95:5) to give 0.180 g (45%) of 1-(3,3,3-trifluoro-2-hydroxy-propyl)-piperidine-3-carbonitrile as an oil.

A solution of 0.180 g (0.81 mmole) of 1-(3,3,3-trifluoro-2-hydroxypropyl)-piperidine-3-carbonitrile from above, 0.137 g (0.86 mmol) of 4-chlorophenyl isocyanate, 0.15 mL of triethylamine and 5 mL of dichloromethane was stirred at room temperature for 16 hours and then concentrated under reduced pressure. The crude material was purified by chromatography on silica gel, to give 0.110 g (36%) of (4-chlorophenyl)-carbamic acid 1-(3-cyanopiperidin-1-ylmethyl)-2,2,2-trifluoroethyl ester as an oil. LCMS MH+=376.

### Example 14

### (4-Chlorophenyl)-carbamic acid 1-(3-benzyloxypiperidin-1-ylmethyl)-2,2,2-trifluoroethyl ester

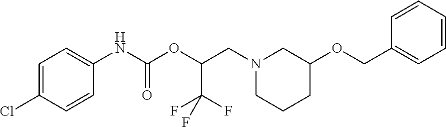 [SMILES: O=C(Nc1ccc(Cl)cc1)OC(CN1CCCC(OCc2ccccc2)C1)C(F)(F)F]

A stirred mixture of 0.200 g (0.88 mmol) of 3-benzyloxypiperidine hydrochloride, 0.856 g (2.6 mmol) of cesium carbonate, 0.147 g (1.3 mmol) of (S)-2-(trifluoromethyl)oxirane and 25 mL of tetrahydrofuran was heated in a sealed tube at 80 degrees for 16 hours. The mixture was filtered and then concentrated under reduced pressure. The crude material was purified by chromatography on silica gel, eluting with dichloromethane-methanol (gradient 98:2-95:5) to give 0.190 g (71%) of 3-(3-benzyloxypiperidinl-yl)-1,1,1-trifluoro-propan-2-ol as a colorless liquid.

A solution of 0.190 g (0.63 mmole) of 3-(3-benzyloxypiperidinl-yl)-1,1,1-trifluoro-propan-2-ol, 0.106 g (0.69 mmol) of 4-chlorophenyl isocyanate, 0.11 mL of triethylamine and 5 mL of dichloromethane was stirred at room temperature for 16 hours and then concentrated under reduced pressure. The crude material was purified by chromatography on silica gel, to give 0.110 g (38%) of (4-chlorophenyl)-carbamic acid 1-(3-benzyloxypiperidin-1-ylmethyl)-2,2,2-trifluoroethyl ester as a light yellow solid. LCMS MH+=457.

### Example 15

### (4-chlorophenyl)-carbamic acid 2,2,2-trifluoro-1-(3-trifluoromethylpiperidin-1-ylmethyl)-ethyl ester

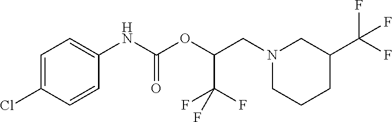 [SMILES: O=C(Nc1ccc(Cl)cc1)OC(CN1CCCC(C(F)(F)F)C1)C(F)(F)F]

Prepared by a similar procedure to example 13 except substituting 3-trifluoromethylpiperidine for piperidine-3-carbonitrile afforded 0.060 g (38%) of (4-chlorophenyl)-carbamic acid 2,2,2-trifluoro-1-(3-trifluoromethylpiperidin-1-ylmethyl)-ethyl ester as a white sticky solid. LCMS MH+=419.

### Example 16

### (4-Chlorophenyl)-carbamic acid 2,2,2-trifluoro-1-(3-methoxypiperidin-1-ylmethyl)-ethyl ester

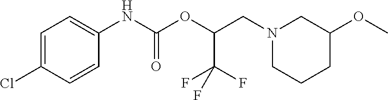 [SMILES: COC1CCCN(CC(OC(=O)Nc2ccc(Cl)cc2)C(F)(F)F)C1]

Prepared by a similar procedure to example 14 except substituting 3-methoxypiperidine hydrochloride for 3-benzyloxypiperidine hydrochloride afforded 0.160 g (48%) of (4-chlorophenyl)-carbamic acid 2,2,2-trifluoro-1-(3-methoxypiperidin-1-ylmethyl)-ethyl ester as a white sticky solid. LCMS MH+=381.

### Example 17

### (4-Chlorophenyl)-carbamic acid 1-(3-ethylpiperidin-1-ylmethyl)-2,2,2-trifluoroethyl ester

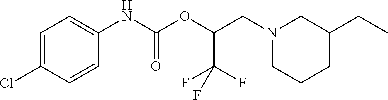 [SMILES: CCC1CCCN(CC(OC(=O)Nc2ccc(Cl)cc2)C(F)(F)F)C1]

Prepared by a similar procedure to example 14 except substituting 3-ethylpiperidine hydrochloride for 3-benzyloxypiperidine hydrochloride afforded 0.185 g (55%) of (4-chlorophenyl)-carbamic acid 1-(3-ethylpiperidin-1-ylmethyl)-2,2,2-trifluoroethyl ester as a white sticky solid. LCMS MH+=379.

### Example 18

### (4-Chlorophenyl)-carbamic acid 1-(3-cyclohexylpiperidin-1-ylmethyl)-2,2,2-trifluoroethyl ester

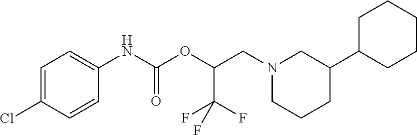 [SMILES: O=C(Nc1ccc(Cl)cc1)OC(CN1CCCC(C2CCCCC2)C1)C(F)(F)F]

Prepared by a similar procedure to example 14 except substituting 3-cyclohexylpiperidine hydrochloride for 3-benzyloxypiperidine hydrochloride afforded 0.120 g (52%) of (4-chlorophenyl)-carbamic acid 1-(3-cyclohexylpiperidin-1-ylmethyl)-2,2,2-trifluoroethyl ester as a yellow sticky solid. LCMS MH+=433.

### Example 19

### (4-Chlorophenyl)-carbamic acid 2,2,2-trifluoro-1-[3-(3-methoxypropyl)-piperidin-1-ylmethyl]-ethyl ester

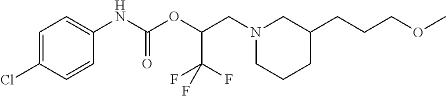 [SMILES: COCCCC1CCCN(CC(OC(=O)Nc2ccc(Cl)cc2)C(F)(F)F)C1]

A mixture of 0.300 g (1.9 mmol) of 3-(3-methoxypropyl)-piperidine, 0.321 g (2.87 mmol) of 2-(trifluoromethyl)oxirane and 25 mL of tetrahydrofuran was stirred for 16 hours at room temperature and then concentrated under reduced pressure. The crude material was purified by chromatography on silica gel, eluting with dichloromethane-methanol (gradient 98:2-95:5) to give 0.400 g (78%) of 1,1,1-trifluoro-3-[3-(3-methoxy-propyl)-piperidin-1-yl]-propan-2-ol as a colorless liquid.

A solution of 0.200 g (0.74 mmole) of 1,1,1-trifluoro-3-[3-(3-methoxypropyl)-piperidin-1-yl]-propan-2-ol, 0.126 g (0.82 mmol) of 4-chlorophenyl isocyanate, 0.13 mL of triethylamine and 5 mL of dichloromethane was stirred at room temperature for 16 hours and then concentrated under reduced pressure. The crude material was purified by chromatography on silica gel, to give 0.230 g (73%) of (4-chlorophenyl)-carbamic acid 2,2,2-trifluoro-1-[3-(3-methoxypropyl)-piperidin-1-ylmethyl]-ethyl ester as a sticky yellow solid. LCMS MH+=423.

### Example 20

### (4-Chlorophenyl)-carbamic acid 2,2,2-trifluoro-1-[3-(4-methoxybenzyl)-piperidin-1-ylmethyl]-ethyl ester

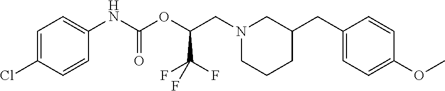 [SMILES: COc1ccc(CC2CCCN(C[C@H](OC(=O)Nc3ccc(Cl)cc3)C(F)(F)F)C2)cc1]

To a round bottomed flask containing 0.616 g (3 mmol) of 3-(4-methoxybenzyl)-piperidine was added ca. 0.336 g (3 mmol) of (S)-2-(trifluoromethyl)oxirane, dropwise over 1 minute (exothermic). After 90 minutes, the mixture was dissolved in acetonitrile and volatiles were removed under reduced pressure to give 1.003 g of (S)-1,1,1-trifluoro-3-[3-(4-methoxybenzyl)-piperidin-1-yl]-propan-2-ol as a light amber oil.

To the (S)-1,1,1-trifluoro-3-[3-(4-methoxybenzyl)-piperidin-1-yl]-propan-2-ol, prepared above, was added 0.461 g (3 mmol) of 4-chlorophenyl isocyanate and ca 0.3 mL of acetonitrile. The mixture was heated at 85 degrees for 90 minutes, cooled, and volatiles removed under reduced pressure. The crude material was purified by chromatography on silica gel, eluting with hexanes-ethyl acetate (90:10) to give 1.142 g (81%) of (4-chlorophenyl)-carbamic acid (S)-2,2,2-trifluoro-1-[3-(4-methoxybenzyl)-piperidin-1-ylmethyl]-ethyl ester as a white foam. LCMS MH+=471.

### Example 21

### (4-Chlorophenyl)-carbamic acid (S)-1-[3-(3,5-dimethoxyphenyl)-piperidin-1-ylmethyl]-2,2,2-trifluoroethyl ester

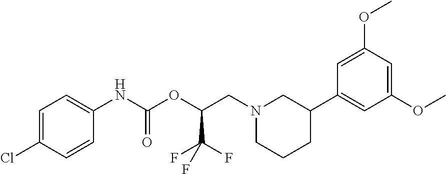 [SMILES: COc1cc(OC)cc(C2CCCN(C[C@H](OC(=O)Nc3ccc(Cl)cc3)C(F)(F)F)C2)c1]

### Step 1

A solution of 2.989 g (15 mmol) of N-Boc-3-piperidone in 20 mL of tetrahydrofuran was added dropwise over 10 minutes to an ice bath cooled solution of 18 mL of a 1M of 3,5-dimethoxyphenyl magnesium bromide in tetrahydrofuran. The mixture was stirred for 4.5 hours, during which time the bath temperature was allowed to warm to ambient. The mixture was poured onto ice cold saturated ammonium chloride solution, extracted twice with ethyl acetate. The combined ethyl acetate layers were washed with water, and then brine, dried over anhydrous sodium sulfate, filtered and concentrated under reduced pressure. Purification by chromatography on silica gel, eluting with hexanes-ethyl acetate (80:20), gave 3.841 g (75%) of 3-(3,5-dimethoxyphenyl)-3-hydroxypiperidine-1-carboxylic acid tert-butyl ester as an oil.

### Step 2

To an ice bath cooled mixture of 3.841 g (11.4 mmol) of 3-(3,5-dimethoxyphenyl)-3-hydroxy-piperidine-1-carboxylic acid tert-butyl ester, 5.885 g (46 mmol) ethyldiisopropylamine and 75 mL of dichloromethane was added ca. 2.608 g of methanesulfonyl chloride. After 2 hours the mixture was poured into water, and extracted twice with ethyl acetate. The combined ethyl acetate layers were washed successively with saturated sodium bicarbonate, water, and brine, dried over anhydrous sodium sulfate, filtered and concentrated under reduced pressure. Purification by chromatography on silica gel, eluting with hexanes-ethyl acetate (90:10), gave 2.014 g (55%) of an oil which was a 1:1 mixture of 5-(3,5-dimethoxyphenyl)-3,6-dihydro-2H-pyridine-1-carboxylic acid tert-butyl ester and 5-(3,5-dimethoxyphenyl)-3,4-dihydro-2H-pyridine-1-carboxylic acid tert-butyl ester.

### Step 3

A solution of 2.014 g (6.3 mmol) of the 1:1 mixture of 5-(3,5-dimethoxyphenyl)-3,6-dihydro-2H-pyridine-1-carboxylic acid tert-butyl ester and 5-(3,5-dimethoxyphenyl)-3,4-dihydro-2H-pyridine-1-carboxylic acid tert-butyl ester from step 2, 0.4 g of 5% palladium on carbon and 50 mL of ethyl acetate was stirred under an atmosphere of hydrogen. The catalyst was removed by filtration and the filtrate concentrated under reduced pressure. Purification by chromatography on silica gel, eluting with hexanes-ethyl acetate (90:10), gave 0.639 g (32%) of 3-(3,5-dimethoxyphenyl)-piperidine-1-carboxylic acid tert-butyl ester as an oil.

### Step 4

To a solution of 0.634 g (1.97 mmol) of 3-(3,5-dimethoxyphenyl)-piperidine-1-carboxylic acid tert-butyl ester in 3 mL of dichloromethane, was added 3 mL of dichloromethane-trifluoroacetic acid (1:2). After 8.5 hours, volatiles were removed under reduced pressure. The residue was mixed with 50 mL of 2 M sodium hydroxide, and extracted with dichloromethane. The dichloromethane layer was dried over anhydrous sodium sulfate, filtered and concentrated under reduced pressure to give 0.407 g of 3-(3,5-dimethoxyphenyl)-piperidine as an oil.

### Step 5

To a round bottomed flask containing 0.402 g (1.8 mmol) of 3-(3,5-dimethoxyphenyl)-piperidine was added ca. 0.244 g (2.2 mmol) of (S)-2-(trifluoromethyl)oxirane dropwise over 15 seconds (exothermic). After 90 minutes, the mixture was dissolved in acetonitrile and volatiles were removed under reduced pressure to give 0.600 g (99%) of (S)-3-[3-(3,5-dimethoxyphenyl)-piperidin-1-yl]-1,1,1-trifluoropropan-2-ol as a light amber oil.

### Step 6

To 0.599 g (1.8 mmol) of (S)-3-[3-(3,5-dimethoxyphenyl)-piperidin-1-yl]-1,1,1-trifluoropropan-2-ol, was added 0.276 g (1.8 mmol) of 4-chlorophenyl isocyanate and ca 0.3 mL of acetonitrile. The mixture was heated at 85 degrees for 90 minutes, cooled, and volatiles removed under reduced pressure. The crude material was purified by chromatography on silica gel, eluting with hexanes-ethyl acetate (80:20) to give 0.736 g (84%) of (4-chlorophenyl)-carbamic acid (S)-1-[3-(3,5-dimethoxyphenyl)-piperidin-1-ylmethyl]-2,2,2-trifluoroethyl ester as a light amber foam. LCMS MH+=487.

### Example 22

### Racemic (4-Chlorophenyl)-carbamic acid 2,2,2-trifluoro-1-[(S)-3-(pyridin-4-yloxy)-piperidin-1-ylmethyl]-ethyl ester and Racemic (4-Chlorophenyl)-carbamic acid 2,2,2-trifluoro-1-[(R)-3-(pyridin-4-yloxy)-piperidin-1-ylmethyl]-ethyl ester

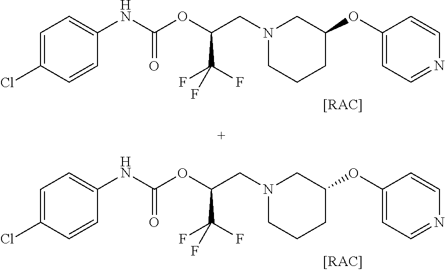 [SMILES: O=C(Nc1ccc(Cl)cc1)O[C@@H](CN1CCC[C@H](Oc2ccncc2)C1)C(F)(F)F ; O=C(Nc1ccc(Cl)cc1)O[C@@H](CN1CCC[C@@H](Oc2ccncc2)C1)C(F)(F)F]

### Step 1

A stirred mixture of 0.200 g (0.79 mmol) of 4-(piperidin-3-yloxy)-pyridine dihydrochloride, 1.03 g (3.2 mmol) of cesium carbonate, 0.133 g (1.2 mmol) of 2-(trifluoromethyl)oxirane and 25 mL of tetrahydrofuran was heated in a sealed tube at 80 degrees for 16 hours. The mixture was filtered and then concentrated under reduced pressure to give 0.200 g (87%) of 1,1,1-trifluoro-3-[3-(pyridin-4-yloxy)-piperidin-1-yl]-propan-2-ol as a yellow liquid.

### Step 2

A solution of 0.100 g (0.35 mmole) of 1,1,1-trifluoro-3-[3-(pyridin-4-yloxy)-piperidin-1-yl]-propan-2-ol, prepared above, 0.058 g (0.38 mmol) of 4-chlorophenyl isocyanate, 0.06 mL of triethylamine and 5 mL of dichloromethane was stirred at room temperature for 16 hours and then concentrated under reduced pressure. The crude material was purified by chromatography on silica gel, to give 0.025 g of racemic (4-chlorophenyl)-carbamic acid 2,2,2-trifluoro-1-[(S)-3-(pyridin-4-yloxy)-piperidin-1-ylmethyl]-ethyl ester (Example 22b) as a white solid (LCMS MH+=444) and 0.015 g racemic (4-chloro-phenyl)-carbamic acid 2,2,2-trifluoro-1-[(R)-3-(pyridin-4-yloxy)-piperidin-1-ylmethyl]-ethyl ester (Example 22a) as a white solid (LCMS MH+=444).

### Example 23

### (4-Chlorophenyl)-carbamic acid (S)-1-[(R)-3-(3,5-bis-trifluoromethylphenyl)-piperidin-1ylmethyl]-2,2,2-trifluoroethyl ester

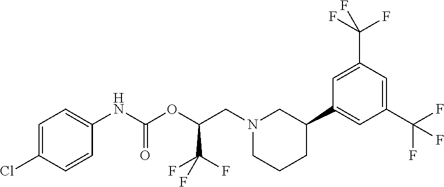 [SMILES: O=C(Nc1ccc(Cl)cc1)O[C@@H](CN1CCC[C@H](c2cc(C(F)(F)F)cc(C(F)(F)F)c2)C1)C(F)(F)F]

### Step 1

Under an atmosphere of argon, a mixture of 1.758 g (6 mmol) of 3,5-bis(trifluoromethyl)bromobenzene, 1.908 g (18 mmol) of sodium carbonate, 0.505 g (0.7 mmol) of bis triphenylphosphine palladium dichloride, 0.885 g (7.2 mmol) of 3-pyridine boronic acid and 120 mL of dioxane-water (10:1) was stirred and heated at 95 degrees for 14 hours. The mixture was cooled, taken up in hexanes-ethyl acetate (1:1) and washed with saturated sodium bicarbonate, brine, and then dried over anhydrous sodium sulfate, filtered and concentrated under reduced pressure. Purification by chromatography on silica gel, eluting with hexanes-ethyl acetate (75:25), gave 1.006 g (58%) of 3-(3,5-bis-trifluoromethylphenyl)-pyridine as a white solid.

### Step 2

A mixture of 0.990 g (3.4 mmol) of 3-(3,5-bis-trifluoromethylphenyl)-pyridine, 0.232 g platinum (IV) oxide, 3.6 mL of hydrochloric acid and 31.4 mL of methanol was hydrogenated in a Parr shaker apparatus at 48 psi for 8 hours. The catalyst was removed by filtration and the filtrate concentrated under reduced pressure. The residue was made basic by the addition of 2M sodium hydroxide, and the mixture extracted twice with dichloromethane. The combined dichloromethane layers were washed with brine, dried over anhydrous sodium sulfate, filtered and concentrated under reduced pressure to give 1.031 g (100%) of 3-(3,5-bis-trifluoromethylphenyl)-piperidine as an oil.

### Step 3

To a round bottomed flask containing 0.535 g (1.8 mmol) of 3-(3,5-bis-trifluoromethylphenyl)-piperidine was added ca. 0.242 g (2.2 mmol) of (S)-2-(trifluoromethyl)oxirane, dropwise over 15 seconds (exothermic). After 90 minutes, the mixture was dissolved in acetonitrile and volatiles were removed under reduced pressure to give 0.738 g (100%) of (S)-3-[3-(3,5-bis-trifluoromethyl-phenyl)-piperidin-1-yl]-1,1,1-trifluoro-propan-2-ol as an off white solid.

### Step 4

To 0.491 g (1.2 mmol) of (S)-3-[3-(3,5-bis-trifluoromethyl-phenyl)-piperidin-1-yl]-1,1,1-trifluoro-propan-2-ol, was added 0.184 g (1.2 mmol) of 4-chlorophenyl isocyanate and ca 0.3 mL of acetonitrile. The mixture was heated at 85 degrees for 90 minutes, cooled, and volatiles removed under reduced pressure. The crude material was purified by chromatography on silica gel, eluting with hexanes-ethyl acetate (90:10) and collecting the front-running peak, to afford 0.229 g (34%) of (4-chlorophenyl)-carbamic acid (S)-1-[(R)-3-(3,5-bis-trifluoromethylphenyl)-piperidin-1ylmethyl]-2,2,2-trifluoroethyl ester as a white solid. LCMS MH+=563.

### Example 24

### (4-Chlorophenyl)-carbamic acid (S)-1-[(S)-3-(3,5-bis-trifluoromethyl-phenyl)-piperidin-1ylmethyl]-2,2,2-trifluoroethyl ester

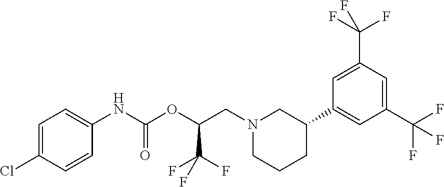 [SMILES: O=C(Nc1ccc(Cl)cc1)O[C@@H](CN1CCC[C@@H](c2cc(C(F)(F)F)cc(C(F)(F)F)c2)C1)C(F)(F)F]

(4-Chlorophenyl)-carbamic acid (S)-1-[(S)-3-(3,5-bis-trifluoromethylphenyl)-piperidin-1ylmethyl]-2,2,2-trifluoroethyl ester (0.193 g, 29%) was prepared according to the methods described for Example 23 except isolating the late-running peak from the flash chromatography as a white foam. LCMS MH+=563.

### Example 25

### Racemic (S)—N-(4-Chlorophenyl)-4,4,4-trifluoro-3-[(R)-3-(3-trifluoromethylphenyl)-piperidin-1-ylmethyl]-butyramide

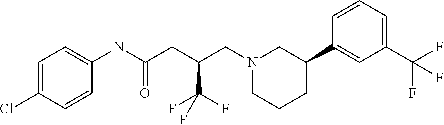 [SMILES: O=C(C[C@@H](CN1CCC[C@H](c2cccc(C(F)(F)F)c2)C1)C(F)(F)F)Nc1ccc(Cl)cc1]

### Step 1

A mixture of 4.203 g (25 mmol) of ethyl 4,4,4-trifluorocrotonate, 20 mL of nitromethane and ca. 0.576 g (5 mmol) of tetramethyl guanidine was stirred for 13 hours at room temperature, and then diluted with water and acidified by the addition of 0.5 M sulfuric acid. The mixture was extracted three times with diethyl ether. The combined ether extracts were washed with water, and then brine, dried over anhydrous sodium sulfate, filtered and concentrated under reduced pressure to give 5.601 g (98%) of 4,4,4-trifluoro-3-nitromethyl-butyric acid ethyl ester as an amber oil.

### Step 2

A mixture of 0.309 g (5.5 mmol) of potassium hydroxide and 50 mL of water was cooled in an ice bath and 1.246 g (5 mmol) of 4,4,4-trifluoro-3-nitromethyl-butyric acid ethyl ester in 10 mL of tetrahydrofuran was added over 5 minutes. The mixture was stirred for 20 minutes, then 0.692 (5.75 mmol) g of magnesium sulfate in 10 mL of water was added, followed by 0.632 g (4 mmol) of potassium permanganate in 50 mL of water was added over 8 minutes. The mixture was stirred for 20 minutes, filtered through celite, washing with dichloromethane. The dichloromethane layer of the filtrate was washed with brine, dried over anhydrous sodium sulfate, filtered and concentrated under reduced pressure. Distillation of the residue at 8 mm Hg, (bath temperature 25-65 degrees) gave 0.231 g (23%) of 4,4,4-trifluoro-3-formyl-butyric acid ethyl ester.

### Step 3

A mixture of 0.352 g (1.78 mmol) of 4,4,4-trifluoro-3-formyl-butyric acid ethyl ester, 0.611 g (2.67 mmol) of 3-(3-(trifluoromethyl)phenyl)piperidine, ca. 0.213 g (3.55 mmol) of acetic acid, 0.753 g (3.55 mmol) of sodium triacetoxyborohydride and 6 mL of dichloromethane was stirred at room temperature for 13 hours. The reaction mixture was taken up in ethyl acetate, washed successively with 0.5 M sodium carbonate, water, and brine. The ethyl acetate layer was dried over anhydrous sodium sulfate, filtered and concentrated under reduced pressure. The crude material was purified by chromatography on silica gel, eluting with hexanes-ethyl acetate (92:8) to give 0.354 g (48%) of 4,4,4-trifluoro-3-[3-(3-trifluoromethyl-phenyl)-piperidin-1-ylmethyl]-butyric acid ethyl ester as an oil.

### Step 4

A mixture of 0.353 g (0.86 mmol) of 4,4,4-trifluoro-3-[3-(3-trifluoromethylphenyl)-piperidin-1-ylmethyl]-butyric acid ethyl ester, 8 mL ethanol and ca. 0.644 mL of 2 M sodium hydroxide solution was heated at reflux and stirred. After 25 minutes, volatiles were removed under reduced pressure. The mixture was treated with 40 mL of saturated sodium dihydrogen phosphate solution, and extracted twice with ethyl acetate. The combined ethyl acetate layers were washed with brine, dried over anhydrous sodium sulfate, filtered and concentrated under reduced pressure to give 0.314 g (95%) of 4,4,4-trifluoro-3-[3-(3-trifluoromethylphenyl)-piperidin-1-ylmethyl]-butyric acid as a glass.

### Step 5

To a solution of 0.312 g (0.81 mmol) of 4,4,4-trifluoro-3-[3-(3-trifluoromethylphenyl)-piperidin-1-ylmethyl]-butyric acid, 0.002 g of dimethylformamide and 6 mL of dichloromethane was added solution of 0.310 g (2.4 mmol) of oxalyl chloride. After 90 minutes the mixture was concentrated under reduced pressure to give the corresponding acid chloride as a yellow foam. To this residue was added 0.130 g (1 mmol) of 4-chloro-aniline, 5 mL of dichloromethane, followed by a solution of ca. 0.514 g (6.5 mmol) of pyridine in 3 mL of dichloromethane. The mixture was stirred at room temperature for 5 hours, and then diluted with ethyl acetate, washed successively with saturated sodium bicarbonate, water, brine, dried over anhydrous sodium sulfate, filtered and concentrated under reduced pressure. The crude material was purified by chromatography on silica gel, eluting with hexanes-ethyl acetate (82:18) and collecting the front-running peak, to afford 0.104 g (26%) of racemic (S)—N-(4-chlorophenyl)-4,4,4-trifluoro-3-[(R)-3-(3-trifluoromethylphenyl)-piperidin-1-ylmethyl]-butyramide as a yellow foam. LCMS MH+=493.

### Example 26

Racemic (S)—N-(4-Chloro-phenyl)-4,4,4-trifluoro-3-[(S)-3-(3-trifluoromethyl-phenyl)-piperidin-1-ylmethyl]-butyramide

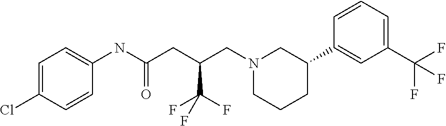 [SMILES: O=C(C[C@@H](CN1CCC[C@@H](c2cccc(C(F)(F)F)c2)C1)C(F)(F)F)Nc1ccc(Cl)cc1]

Racemic (S)—N-(4-Chloro-phenyl)-4,4,4-trifluoro-3-[(S)-3-(3-trifluoromethyl-phenyl)-piperidin-1-ylmethyl]-butyramide (0.111 g, 28%) was prepared according to the methods described for Example 25 except isolating the late-running peak from the flash chromatography as a glass. LCMS MH+=493.

### Example 27

### (4-Chlorophenyl)-carbamic acid 2,2,2-trifluoro-1-(4-phenyl-azepan-1-ylmethyl)-ethyl ester

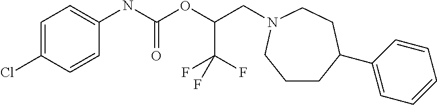 [SMILES: O=C(Nc1ccc(Cl)cc1)OC(CN1CCCC(c2ccccc2)CC1)C(F)(F)F]

Prepared by a similar procedure to example 1 except substituting 4-phenyl-azepane for 3-(2-methoxyphenyl)-piperidine afforded 0.578 g (87%) of (4-chlorophenyl)-carbamic acid 2,2,2-trifluoro-1-(4-phenyl-azepan-1-ylmethyl)-ethyl ester as a white solid. LCMS MH+=441.

### Example 28

### (4-Chlorophenyl)-carbamic acid (S)-2,2,2-trifluoro-1-[3-(3-methyl-[1,2,4]oxadiazol-5-yl)-piperidin-1-ylmethyl]-ethyl ester hydrochloride

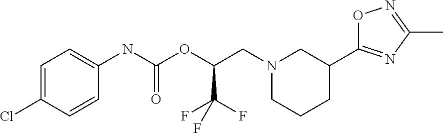 [SMILES: Cc1noc(C2CCCN(C[C@H](OC(=O)Nc3ccc(Cl)cc3)C(F)(F)F)C2)n1]

In a 25 mL round-bottom flask was placed (S)-2-(trifluoromethyl)oxirane (350 mg, 3.12 mmol), followed by 3-methyl-5-(piperidin-3-yl)-1,2,4-oxadiazole (Alfa, 501 mg, 3.00 mmol). Dichloromethane (3 mL) was added. The resultant slurry was stirred at room temperature for 30 minutes. Diisopropylethylamine (DIPEA) (404 mg, 546 μl, 3.12 mmol) was added and the suspension was stirred at room temperature for 2.5 h, at which point the reaction had gone into solution. No starting piperidine remained by LCMS. The crude mixture was concentrated and purified by flash chromatography (silica gel, 24 g, 5% to 15% ethyl acetate in hexanes eluent) to afford 560 mg (64%) of (S)-1,1,1-trifluoro-3-[3-(3-methyl-[1,2,4]oxadiazol-5yl)-piperidin-1-yl]-propan-2-ol, which was used as is in the next step. LCMS MH+=280.

In a 50 mL round-bottom flask, (2S)-1,1,1-trifluoro-3-(3-(3-methyl-1,2,4-oxadiazol-5-yl)piperidin-1-yl)propan-2-ol (559 mg, 2.00 mmol) was combined with acetonitrile (15.0 ml) to give a colorless solution. 1-Chloro-4-isocyanatobenzene (307 mg, 2.00 mmol) was added. The reaction mixture was warmed at 85° C. for 3 h and then concentrated. The crude material was purified by flash chromatography (silica gel, 40 g, 10%, then 10% to 15% ethyl acetate in hexanes eluent) to give 746 mg (87%) of (4-chlorophenyl)carbamic acid (S)-2,2,2-trifluoro-1-[3-(3-methyl-[1,2,4]oxadiazol-5-yl)-piperidin-1-ylmethyl]-ethyl ester as a 1:1 mixture of epimers. This material was taken up in 30 mL of ether. To that was added 3 mL of 1 M HCl in ether. The resultant white sticky solid was broken up, filtered, washed with ether and dried under vacuum to afford 760 mg of (4-chlorophenyl)carbamic acid (S)-2,2,2-trifluoro-1-[3-(3-methyl-[1,2,4]oxadiazol-5-yl)-piperidin-1-ylmethyl]-ethyl ester hydrochloride as a white solid. LCMS MH+=433.

### Example 29

### (4-Chlorophenyl)-carbamic acid (S)-2,2,2-trifluoro-1-[3-(3-trifluoromethylphenyl)-piperidin-1-ylmethyl]-ethyl ester hydrochloride

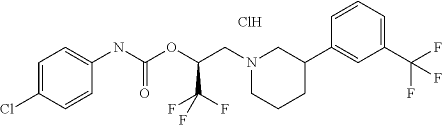 [SMILES: O=C(Nc1ccc(Cl)cc1)O[C@@H](CN1CCCC(c2cccc(C(F)(F)F)c2)C1)C(F)(F)F ; Cl]

To a stirred solution of 3-(3-trifluoromethylphenyl)-piperidine hydrochloride (250 mg, 0.94 mmol) in dry acetonitrile (3 mL) was added diisoproylethyl amine (365 mg, 2.82 mmol). After stirring for 10 minutes, (S)-2-(trifluoromethyl)oxirane (316 mg, 2.82 mmol) was added. The mixture was stirred for 3 h and then concentrated in vacuo. The residue was purified by flash chromatography (10% ethyl acetate/dichloromethane eluent) to provide (S)-1,1,1-trifluoro-3-[3-(3-trifluoromethylphenyl)-piperidin-1-yl]-propan-2-ol as a colorless oil (279 mg, 87%). MH+=342.

A mixture of (S)-1,1,1-trifluoro-3-[3-(3-trifluoromethylphenyl)-piperidin-1-yl]-propan-2-ol, prepared above, (279 mg, 0.82 mmol) and 1-chloro-4-isocyanatobenzene (138 mg, 0.90 mmol) in acetonitrile (3 mL) was stirred overnight. The reaction mixture was concentrated in vacuo. The residue was purified by flash chromatography (30/1 dichloromethane/ethyl acetate eluent) to provide (4-chlorophenyl)-carbamic acid (S)-2,2,2-trifluoro-1-[3-(3-trifluoromethylphenyl)-piperidin-1-ylmethyl]-ethyl ester as a sticky oil. 1N HCl in ethyl ether (1 ml) was added to provide the corresponding HCl salt (200 mg, 49%) as a white solid. LCMS MH+=495.

### Example 30

### (4-Chlorophenyl)-carbamic acid (S)-2,2,2-trifluoro-1-[(R)-3-(3-trifluoromethylphenyl)-piperidin-1-ylmethyl]-ethyl ester hydrochloride

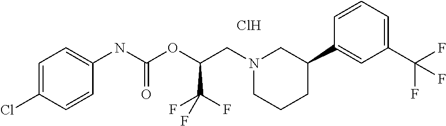 [SMILES: O=C(Nc1ccc(Cl)cc1)O[C@@H](CN1CCC[C@H](c2cccc(C(F)(F)F)c2)C1)C(F)(F)F ; Cl]

The diastereomeric mixture of (4-chlorophenyl)-carbamic acid (S)-2,2,2-trifluoro-1-[3-(3-trifluoromethyl-phenyl)-piperidin-1-ylmethyl]-ethyl ester (225 mg, 453 μmol), prepared in example 29, was separated by SFC chromatography (Pirkel's Whelk chiral column) to provide (4-chlorophenyl)carbamic acid (S)-2,2,2-trifluoro-1-[(R)-3-(3-trifluoromethyl-phenyl)-piperidin-1-ylmethyl]-ethyl ester (34%) as the late-running peak. 1M HCl in ether was added to provide the corresponding HCl salt (87 mg) as a white solid. LCMS MH+=495.

### Example 31

### (4-Chlorophenyl)-carbamic acid (S)-2,2,2-trifluoro-1-[3-(4-fluoro-3-methoxyphenyl)-piperidin-1-ylmethyl]-ethyl ester hydrochloride

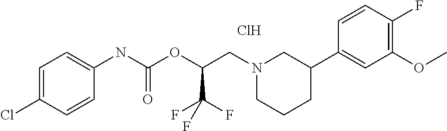 [SMILES: COc1cc(C2CCCN(C[C@H](OC(=O)Nc3ccc(Cl)cc3)C(F)(F)F)C2)ccc1F ; Cl]

Prepared by a similar procedure to example 29 except substituting 3-(4-fluoro-3-methoxyphenyl)-piperidine hydrochloride for 3-(3-trifluoromethylphenyl)-piperidine hydrochloride afforded (4-chlorophenyl)-carbamic acid (S)-2,2,2-trifluoro-1-[3-(4-fluoro-3-methoxyphenyl)-piperidin-1-ylmethyl]-ethyl ester hydrochloride as a white solid. LCMS MH+=475.

### Example 32

### (4-Chloro-phenyl)-carbamic acid (S)-2,2,2-trifluoro-1-[(R)-3-(4-fluoro-3-methoxy-phenyl)-piperidin-1-ylmethyl]-ethyl ester hydrochloride

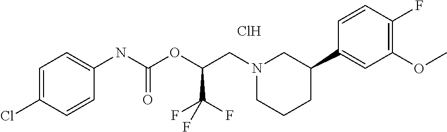 [SMILES: COc1cc([C@H]2CCCN(C[C@H](OC(=O)Nc3ccc(Cl)cc3)C(F)(F)F)C2)ccc1F ; Cl]

Prepared by a similar procedure to example 30 except substituting (4-chlorophenyl)-carbamic acid (S)-2,2,2-trifluoro-1-[3-(4-fluoro-3-methoxyphenyl)-piperidin-1-ylmethyl]-ethyl ester for (4-chlorophenyl)-carbamic acid (S)-2,2,2-trifluoro-1-[3-(3-trifluoromethylphenyl)-piperidin-1-ylmethyl]-ethyl ester afforded (4-chlorophenyl)-carbamic acid (S)-2,2,2-trifluoro-1-[(R)-3-(4-fluoro-3-methoxy-phenyl)-piperidin-1-ylmethyl]-ethyl ester hydrochloride as a white solid. LCMS MH+=475.

### Example 33

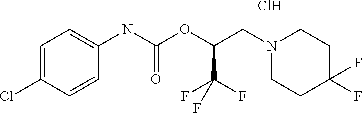 [SMILES: Cl ; O=C(Nc1ccc(Cl)cc1)O[C@@H](CN1CCC(F)(F)CC1)C(F)(F)F]

### (4-Chlorophenyl)-carbamic acid (S)-1-(4,4-difluoropiperidin-1-ylmethyl)-2,2,2-trifluoroethyl ester hydrochloride

In a 2 ml vial, 4,4-difluoropiperidine (150 mg, 1.24 mmol) was combined with acetonitrile (10.0 ml) to give a colorless solution. (S)-2-(trifluoromethyl)oxirane (278 mg, 215 μl, 2.48 mmol) was added and the resulting reaction mixture was stirred at room temperature for 2 hr. The crude reaction mixture was concentrated in vacuo to afford (S)-3-(4,4-difluoropiperidin-1-yl)-1,1,1-trifluoropropan-2-ol (280 mg) as an oil.

In a 10 mL round-bottomed flask, (S)-3-(4,4-difluoropiperidin-1-yl)-1,1,1-trifluoropropan-2-ol (280 mg, 1.2 mmol) was combined with acetonitrile (5.00 ml) to give a colorless solution. 1-chloro-4-isocyanatobenzene (184 mg, 1.2 mmol) was added and the resulting reaction mixture was stirred at room temperature overnight. The crude reaction mixture was concentrated in vacuo and purified on a silica column (hexane to 60% EtOAc/hexane) to afford an oil (300 mg). The oil was dissolved in ether and hexane and 4N HCl solution (0.2 ml) was added. The mixture was concentrated to afford (4-chlorophenyl)-carbamic acid (S)-1-(4,4-difluoropiperidin-1-ylmethyl)-2,2,2-trifluoroethyl ester hydrochloride (155 mg) as a yellow solid. (M+H)⁺=387 m/e.

### Example 34

### (4-Chlorophenyl)-carbamic acid (S)-2,2,2-trifluoro-1-{3-[4-fluoro-3-(2-methoxyethoxy)-phenyl]-piperidin-1-ylmethyl}-ethyl ester

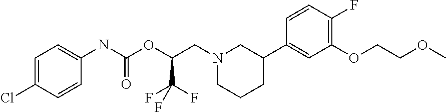 [SMILES: COCCOc1cc(C2CCCN(C[C@H](OC(=O)Nc3ccc(Cl)cc3)C(F)(F)F)C2)ccc1F]

To a stirred solution of 3-[4-fluoro-3-(2-methoxyethoxy)-phenyl]-piperidine hydrochloride (250 mg, 0.987 mmol) in dry acetonitrile (3 mL) was added diisoproylethyl amine (255 mg, 1.97 mmol). After stirring for 10 minutes, (S)-2-(trifluoromethyl)oxirane (332 mg, 2.96 mmol) was added. The mixture was stirred for 3 h and then concentrated in vacuo. The residue was purified by flash chromatography (2.5% methanol/dichloromethane eluent) to provide (S)-1,1,1-trifluoro-3-{3-[4-fluoro-3-(2-methoxyethoxy)-phenyl]-piperidine-1-yl}-propan-2-ol was obtained as an oil (240 mg, 66%). MH+=366.

A mixture of (S)-1,1,1-trifluoro-3-{3-[4-fluoro-3-(2-methoxyethoxy)-phenyl]-piperidine-1-yl}-propan-2-ol prepared above, (240 mg, 0.657 mmol) and 1-chloro-4-isocyanatobenzene (111 mg, 0.723 mmol) in acetonitrile (3 mL) was stirred overnight. The reaction mixture was concentrated in vacuo. The residue was purified by flash chromatography (30/1 dichloromethane/ethyl acetate eluent) to provide (4-chlorophenyl)-carbamic acid (S)-2,2,2-trifluoro-1-{3-[4-fluoro-3-(2-methoxy-ethoxy)-phenyl]-piperidin-1-ylmethyl}-ethyl ester was obtained as a white solid (330 mg, 97%). MH+=519.

### Example 35

### (4-Chlorophenyl)-carbamic acid (S)-2,2,2-trifluoro-1-{(R)-3-[4-fluoro-3-(2-methoxyethoxy)-phenyl]-piperidin-1-ylmethyl}-ethyl ester hydrochloride

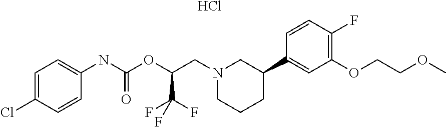 [SMILES: Cl ; COCCOc1cc([C@H]2CCCN(C[C@H](OC(=O)Nc3ccc(Cl)cc3)C(F)(F)F)C2)ccc1F]

Prepared by a similar procedure to example 30 except substituting (4-chlorophenyl)-carbamic acid (S)-2,2,2-trifluoro-1-{3-[4-fluoro-3-(2-methoxy-ethoxy)-phenyl]-piperidin-1-ylmethyl}-ethyl ester for (4-chloro-phenyl)-carbamic acid (S)-2,2,2-trifluoro-1-[3-(3-trifluoromethyl-phenyl)-piperidin-1-ylmethyl]-ethyl ester and using a Daicel AD column instead of a Pirkel's Whelk chiral column afforded 80 mg of (4-chlorophenyl)-carbamic acid (S)-2,2,2-trifluoro-1-{(R)-3-[4-fluoro-3-(2-methoxyethoxy)-phenyl]-piperidin-1-ylmethyl}-ethyl ester hydrochloride as a white solid. LCMS MH+=519.

### Example 36

### (4-Chlorophenyl)-carbamic acid 2,2,2-trifluoro-1-(3-phenylpiperidin-1-ylmethyl)-ethyl ester hydrochloride

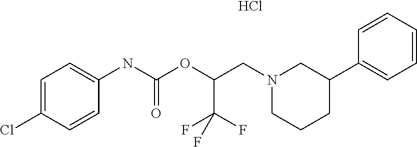 [SMILES: Cl ; O=C(Nc1ccc(Cl)cc1)OC(CN1CCCC(c2ccccc2)C1)C(F)(F)F]

Prepared by a similar procedure to example 2 except substituting 3-phenylpiperidine for 3-(3-methoxyphenyl)piperidine afforded 610 mg of (4-chlorophenyl)-carbamic acid 2,2,2-trifluoro-1-(3-phenylpiperidin-1-ylmethyl)-ethyl ester hydrochloride as a white solid. LCMS MH+=427.

### Example 37

### (4-Chlorophenyl)-carbamic acid 1-[3-(4-chlorophenyl)-piperidin-1-ylmethyl]-2,2,2-trifluoro ethyl ester

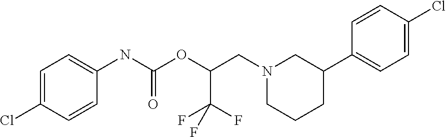 [SMILES: O=C(Nc1ccc(Cl)cc1)OC(CN1CCCC(c2ccc(Cl)cc2)C1)C(F)(F)F]

In a 15 mL round-bottomed flask, 3-(4-chlorophenyl)piperidine (390 mg, 1.99 mmol) was combined with acetonitrile (2.88 ml) and dichloromethane (2.88 mL) to give a colorless solution. 2-(Trifluoromethyl)oxirane (447 mg, 3.99 mmol) was added and the resultant mixture was stirred at room temperature overnight and then concentrated in vacuo to afford 590 mg (96%) of 3-(3-(4-chlorophenyl)piperidin-1-yl)-1,1,1-trifluoropropan-2-ol as a colorless oil. LCMS MH+=308.

In a 20 mL round-bottomed flask, 3-(3-(4-chlorophenyl)piperidin-1-yl)-1,1,1-trifluoropropan-2-ol (590 mg, 1.92 mmol), prepared above, was combined with acetonitrile (8 ml) and dichloromethane (0.3 ml) to give a colorless solution. 1-Chloro-4-isocyanatobenzene (294 mg, 1.92 mmol) was added. The resultant mixture was stirred at room temperature for 60 min and then concentrated in vacuo and triturated with hexane to afford 620 mg (67%) of (4-chlorophenyl)carbamic acid 1-[3-(4-chlorophenyl)-piperidin-1-ylmethyl]-2,2,2-trifluoroethyl ester as a white solid. LCMS MH+=461, 463.

### Example 38

### (4-Chlorophenyl)-carbamic acid (S)-2,2,2-trifluoro-1-[3-(4-methoxyphenyl)-piperidin-1-ylmethyl]-ethyl ester

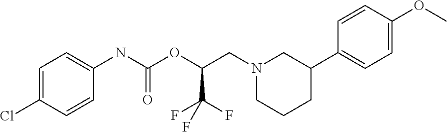 [SMILES: COc1ccc(C2CCCN(C[C@H](OC(=O)Nc3ccc(Cl)cc3)C(F)(F)F)C2)cc1]

In a 1 mL round-bottomed flask, 3-(4-methoxyphenyl)piperidine (200 mg, 1.05 mmol) was combined with acetonitrile (1.00 ml) to give a colorless solution. (S)-2-(Trifluoromethyl)oxirane (200 mg, 1.78 mmol) was added. The resultant reaction mixture was stirred at room temperature overnight and then concentrated in vacuo to afford 310 mg (98%) of (2S)-1,1,1-trifluoro-3-(3-(4-methoxyphenyl)piperidin-1-yl)propan-2-ol as an oil. LCMS MH+=304.

In a 10 mL round-bottomed flask, (2S)-1,1,1-trifluoro-3-(3-(4-methoxyphenyl)piperidin-1-yl)propan-2-ol (305 mg, 1.01 mmol) was combined with acetonitrile (2.00 ml) to give a colorless solution. 1-Chloro-4-isocyanatobenzene (162 mg, 1.06 mmol) was added and the reaction mixture was stirred at room temperature overnight. The crude reaction mixture was concentrated in vacuo and purified by flash chromatography (silica, dichloromethane to 20% ethyl acetate/dichloromethane) to afford 170 mg (35%) of (4-chlorophenyl)-carbamic acid (S)-2,2,2-trifluoro-1-[3-(4-methoxyphenyl)-piperidin-1-ylmethyl]-ethyl ester as a white solid. LCMS MH+=457.

### Example 39

### (4-Chlorophenyl)-carbamic acid (S)-1-(3-benzoylpiperidin-1-ylmethyl)-2,2,2-trifluoroethyl ester hydrochloride

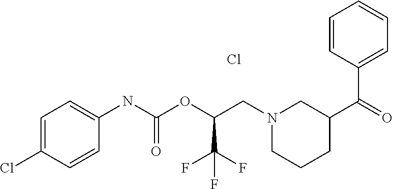 [SMILES: O=C(Nc1ccc(Cl)cc1)O[C@@H](CN1CCCC(C(=O)c2ccccc2)C1)C(F)(F)F ; Cl]

In a 15 mL round-bottomed flask, phenyl(piperidin-3-yl)methanone (100 mg, 528 μmol) was combined with acetonitrile (1.00 ml) to give a colorless suspension and warmed to make it become a solution. (S)-2-(Trifluoromethyl)oxirane (118 mg, 91.8 μl, 1.06 mmol) was added and the reaction was stirred at room temperature for 2 h. The crude reaction mixture was concentrated in vacuo to afford 150 mg (94%) of phenyl(1-((S)-3,3,3-trifluoro-2-hydroxypropyl)piperidin-3-yl)methanone as an oil. LCMS MH+=302.

In a 10 mL round-bottomed flask, phenyl(1-((S)-3,3,3-trifluoro-2-hydroxypropyl)piperidin-3-yl)methanone (150 mg, 0.498 mmol), prepared above, was combined with acetonitrile (2.00 ml) to give a colorless solution. 1-Chloro-4-isocyanatobenzene (80.3 mg, 523 μmol) was added and the reaction was stirred at room temperature overnight. The crude reaction mixture was concentrated in vacuo and purified by flash chromatography (silica, hexane to 20% ethyl acetate/hexane) to give an oil. This oil was dissolved in ether and hexane. A solution of 4N HCl (0.2 ml) was added. The solvent was evaporated to afford (4-chlorophenyl)carbamic acid (S)-1-(3-benzoylpiperidin-1-ylmethyl)-2,2,2-trifluoroethyl ester hydrochloride 163 mg (67%) as a white solid. LCMS MH+=455.

### Example 40

### (4-Chlorophenyl)-carbamic acid (S)-2,2,2-trifluoro-1-(3-phenoxypiperidin-1-ylmethyl)-ethyl ester hydrochloride

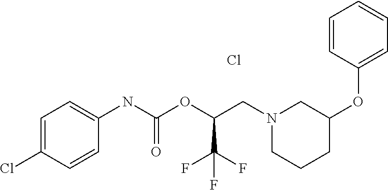 [SMILES: O=C(Nc1ccc(Cl)cc1)O[C@@H](CN1CCCC(Oc2ccccc2)C1)C(F)(F)F ; Cl]

Prepared by a similar procedure to example 39 except substituting 3-phenoxypiperidine for phenyl-(piperidin-3-yl)methanone afforded 380 mg of (4-chlorophenyl)-carbamic acid (S)-2,2,2-trifluoro-1-(3-phenoxypiperidin-1-ylmethyl)-ethyl ester hydrochloride as a white solid. LCMS MH+=443.

### Example 41

### (4-Chlorophenyl)-carbamic acid (S)-2,2,2-trifluoro-1-[3-(3-fluorophenyl)-piperidin-1-ylmethyl]-ethyl ester hydrochloride

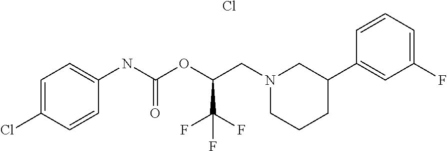 [SMILES: Cl ; O=C(Nc1ccc(Cl)cc1)O[C@@H](CN1CCCC(c2cccc(F)c2)C1)C(F)(F)F]

Prepared by a similar procedure to example 39 except substituting 3-(3-fluorophenyl)piperidine for phenyl-(piperidin-3-yl)methanone afforded 158 mg of (4-chlorophenyl)-carbamic acid (S)-2,2,2-trifluoro-1-[3-(3-fluorophenyl)-piperidin-1-ylmethyl]-ethyl ester hydrochloride as a white solid. LCMS MH+=445.

### Example 42

### (4-Chlorophenyl)-carbamic acid (S)-2,2,2-trifluoro-1-[3-(4-fluorophenyl)-piperidin-1-ylmethyl]-ethyl ester hydrochloride

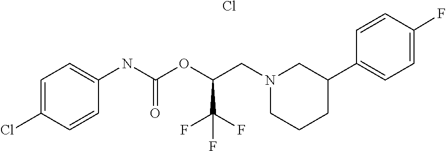 [SMILES: Cl ; O=C(Nc1ccc(Cl)cc1)O[C@@H](CN1CCCC(c2ccc(F)cc2)C1)C(F)(F)F]

Prepared by a similar procedure to example 39 except substituting 3-(4-fluorophenyl)piperidine for phenyl-(piperidin-3-yl)methanone afforded 178 mg of (4-chlorophenyl)-carbamic acid (S)-2,2,2-trifluoro-1-[3-(4-fluorophenyl)-piperidin-1-ylmethyl]-ethyl ester hydrochloride as a white solid. LCMS MH+=445.

### Example 43

### (4-Chlorophenyl)-carbamic acid (S)-1-[3-(3-ethoxyphenyl)-piperidin-1-ylmethyl]-2,2,2-trifluoroethyl ester hydrochloride

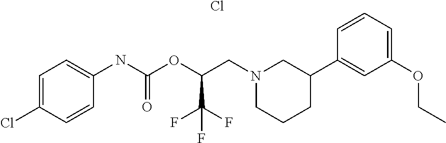 [SMILES: Cl ; CCOc1cccc(C2CCCN(C[C@H](OC(=O)Nc3ccc(Cl)cc3)C(F)(F)F)C2)c1]

### Step 1

In a 100 mL round-bottomed flask (dried), tert-butyl 3-oxopiperidine-1-carboxylate (900 mg, 4.52 mmol) was combined with anhydrous ether (30.0 ml) to give a colorless solution. The reaction was cooled to 0° C. and (3-ethoxyphenyl)magnesium bromide (10.8 ml, 5.42 mmol) was added dropwise. After addition, the reaction mixture was allowed to warm to room temperature and was stirred at this temperature for 2 hr. Reaction mixture was quenched with NH₄Cl (saturated solution) and extracted with ethyl acetate (2×30 ml). The combined organics were washed with brine and water, dried and concentrated in vacuo to give an oil which was purified by flash chromatography (silica, dichloromethane to 50% ethyl acetate/dichloromethane) to afford 560 mg (39%) of tert-butyl 3-(3-ethoxyphenyl)-3-hydroxypiperidine-1-carboxylate as an oil which was used as is for the next step.

### Step 2

In a 15 mL pear-shaped flask, tert-butyl 3-(3-ethoxyphenyl)-3-hydroxypiperidine-1-carboxylate (500 mg, 1.56 mmol) was combined with TFA (4.44 g, 3 mL, 38.9 mmol) to give an orange solution. The reaction mixture was heated at 80° C. for 7 hr, concentrated in vacuo and partitioned between chloroform and saturated NaHCO₃ (10 ml each). The aqueous phase was extracted with chloroform (10 ml). The combined organic layers were dried and concentrated in vacuo to afford 230 mg (51%) of 5-(3-ethoxyphenyl)-1,2,3,6-tetrahydropyridine as an oil. LCMS MH+=204.

### Step 3

In a 200 mL pressure bottle, 5-(3-ethoxyphenyl)-1,2,3,6-tetrahydropyridine (230 mg, 1.13 mmol) and Pd—C (30 mg) were combined with methanol (20 ml) to give a black suspension. The mixture was hydrogenated at room temperature under pressure (30 psi) for 6 hr. The reaction mixture was filtered through celite and washed with methanol. The combined filtrate and washes were concentrated. The crude material was purified by flash chromatography (silica, dichloromethane to 10% methanol/dichloromethane with 1% Et₃N eluent) to afford 220 mg of 3-(3-ethoxyphenyl)piperidine as an orange oil. LCMS MH+=206.

### Step 4

In a 15 mL round-bottomed flask, 3-(3-ethoxyphenyl)piperidine (220 mg, 1.07 mmol) was combined with acetonitrile (1.00 ml) to give a colorless suspension and warmed to make it become a solution. (S)-2-(trifluoromethyl)oxirane (240 mg, 186 μl, 2.14 mmol) was added and the reaction mixture was stirred at room temperature for 2 hr. After stirring at room temperature over the weekend, the reaction was concentrated and purified by flash chromatography (silica, dichloromethane to 60% ethyl acetate/dichloromethane eluent) to afford 70 mg (20%) of (2S)-3-(3-(3-ethoxyphenyl)piperidin-1-yl)-1,1,1-trifluoropropan-2-ol as a colorless oil. LCMS MH+=318.

### Step 5

In a 5 mL pear-shaped flask, (2S)-3-(3-(3-ethoxyphenyl)piperidin-1-yl)-1,1,1-trifluoropropan-2-ol (68 mg, 214 μmol) was combined with acetonitrile (2 ml) to give a colorless solution. 1-Chloro-4-isocyanatobenzene (32.9 mg, 214 μmol) was added and the reaction mixture was stirred at room temperature for 3 hr. The crude reaction mixture was concentrated in vacuo and purified by flash chromatography (silica, dichloromethane to 30% ethyl acetate/dichloromethane eluent) to give the desired product. This material was dissolved in ether/hexane and treated with a solution of 4N HCl in dioxane (0.2 ml). The suspension was concentrated in vacuo to afford 70 mg (52%) of (4-chlorophenyl)-carbamic acid (S)-1-[3-(3-ethoxyphenyl)-piperidin-1-ylmethyl]-2,2,2-trifluoroethyl ester hydrochloride as a white solid. LCMS MH+=471.

### Example 44

### (4-Chlorophenyl)-carbamic acid (S)-2,2,2-trifluoro-1-(3-phenylsulfanyl-piperidin-1-ylmethyl)-ethyl ester hydrochloride

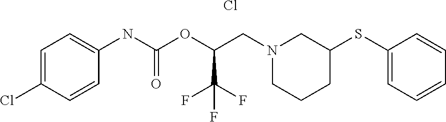 [SMILES: Cl ; O=C(Nc1ccc(Cl)cc1)O[C@@H](CN1CCCC(Sc2ccccc2)C1)C(F)(F)F]

Prepared by a similar procedure to example 39 except substituting 3-(phenylthio)piperidine for phenyl-(piperidin-3-yl)methanone afforded 70 mg of (4-chlorophenyl)-carbamic acid (S)-2,2,2-trifluoro-1-(3-phenylsulfanyl-piperidin-1-ylmethyl)-ethyl ester hydrochloride as a white solid. LCMS MH+=459.

### Example 45

### (4-Chlorophenyl)-carbamic acid (S)-1-[3-(4-chlorophenoxy)-piperidin-1-ylmethyl]-2,2,2-trifluoroethyl ester hydrochloride

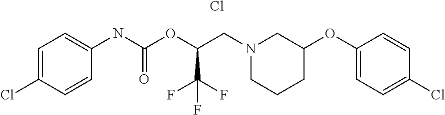 [SMILES: Cl ; O=C(Nc1ccc(Cl)cc1)O[C@@H](CN1CCCC(Oc2ccc(Cl)cc2)C1)C(F)(F)F]

Prepared by a similar procedure to example 39 except substituting 3-(4-chlorophenoxyl)piperidine for phenyl-(piperidin-3-yl)methanone afforded 140 mg of (4-chloro-phenyl)-carbamic acid (S)-1-[3-(4-chlorophenoxy)-piperidin-1-ylmethyl]-2,2,2-trifluoroethyl ester hydrochloride as a white solid. LCMS MH+=477, 479.

### Example 46

### (4-Chlorophenyl)-carbamic acid (S)-1-[3-(4-cyanophenoxy)-piperidin-1-ylmethyl]-2,2,2-trifluoroethyl ester hydrochloride

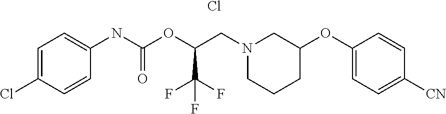 [SMILES: Cl ; N#Cc1ccc(OC2CCCN(C[C@H](OC(=O)Nc3ccc(Cl)cc3)C(F)(F)F)C2)cc1]

Prepared by a similar procedure to example 39 except substituting 3-(4-cyanophenoxyl)piperidine for phenyl-(piperidin-3-yl)methanone afforded 110 mg of (4-chlorophenyl)-carbamic acid (S)-1-[3-(4-cyanophenoxy)-piperidin-1-ylmethyl]-2,2,2-trifluoroethyl ester hydrochloride as a white solid. LCMS MH+=468.

### Example 47

### (4-Chlorophenyl)-carbamic acid (S)-1-[3-(3-cyanophenoxy)-piperidin-1-ylmethyl]-2,2,2-trifluoroethyl ester hydrochloride

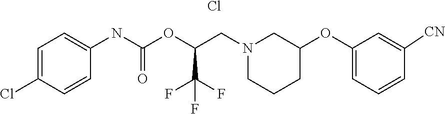 [SMILES: Cl ; N#Cc1cccc(OC2CCCN(C[C@H](OC(=O)Nc3ccc(Cl)cc3)C(F)(F)F)C2)c1]

Prepared by a similar procedure to example 39 except substituting 3-(3-cyanophenoxyl)piperidine for phenyl-(piperidin-3-yl)methanone afforded 85 mg of (4-chlorophenyl)-carb amic acid (S)-1-[3-(3-cyanophenoxy)-piperidin-1-ylmethyl]-2,2,2-trifluoroethyl ester hydrochloride as a white solid. LCMS MH+=468.

### Example 48

### (4-Chlorophenyl)-carbamic acid (S)-1-[3-(4-fluorophenoxy)-piperidin-1-ylmethyl]-2,2,2-trifluoroethyl ester hydrochloride

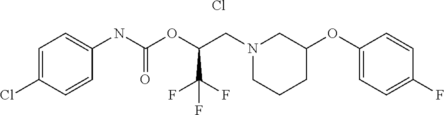 [SMILES: Cl ; O=C(Nc1ccc(Cl)cc1)O[C@@H](CN1CCCC(Oc2ccc(F)cc2)C1)C(F)(F)F]

Prepared by a similar procedure to example 39 except substituting 3-(4-fluorophenoxyl)piperidine for phenyl-(piperidin-3-yl)methanone afforded 188 mg of (4-chlorophenyl)-carbamic acid (S)-1-[3-(4-fluorophenoxy)-piperidin-1-ylmethyl]-2,2,2-trifluoroethyl ester hydrochloride as a white solid. LCMS MH+=461.

### Example 49

### (4-Chlorophenyl)-carbamic acid (S)-1-[3-(3-methoxyphenoxy)-piperidin-1-ylmethyl]-2,2,2-trifluoroethyl ester hydrochloride

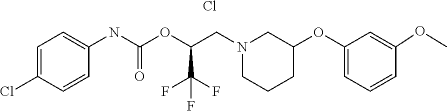 [SMILES: Cl ; COc1cccc(OC2CCCN(C[C@H](OC(=O)Nc3ccc(Cl)cc3)C(F)(F)F)C2)c1]

Prepared by a similar procedure to example 39 except substituting 3-(3-methoxyphenoxyl)piperidine for phenyl-(piperidin-3-yl)methanone afforded 180 mg of (4-chlorophenyl)-carbamic acid (S)-1-[3-(3-methoxyphenoxy)-piperidin-1-ylmethyl]-2,2,2-trifluoroethyl ester hydrochloride as a white solid. LCMS MH+=473.

### Example 50

(4-Chlorophenyl)-carbamic acid (S)-2,2,2-trifluoro-1-[3-(3-trifluoromethoxyphenoxyl)piperidin-1-ylmethyl]-ethyl ester hydrochloride

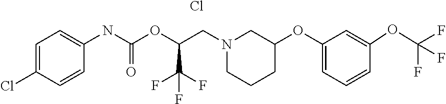 [SMILES: Cl ; O=C(Nc1ccc(Cl)cc1)O[C@@H](CN1CCCC(Oc2cccc(OC(F)(F)F)c2)C1)C(F)(F)F]

Prepared by a similar procedure to example 39 except substituting 3-(3-trifluoromethoxy-phenoxy)-piperidine for phenyl-(piperidin-3-yl)methanone afforded 80 mg of (4-chlorophenyl)-carbamic acid (S)-2,2,2-trifluoro-1-[3-(3-trifluoromethoxyphenoxyl)piperidin-1-ylmethyl]-ethyl ester hydrochloride as a white solid. LCMS MH+=511.

### Example 51

### (4-Chlorophenyl)-carbamic acid (S)-2,2,2-trifluoro-1-[3-(4-fluorophenoxymethyl)-piperidin-1-ylmethyl]-ethyl ester hydrochloride

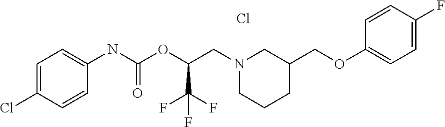 [SMILES: O=C(Nc1ccc(Cl)cc1)O[C@@H](CN1CCCC(COc2ccc(F)cc2)C1)C(F)(F)F ; Cl]

Prepared by a similar procedure to example 39 except substituting 3-((4-fluorophenoxy)methyl)-piperidine for phenyl-(piperidin-3-yl)methanone afforded 180 mg of (4-chlorophenyl)-carbamic acid (S)-2,2,2-trifluoro-1-[3-(4-fluorophenoxymethyl)-piperidin-1-ylmethyl]-ethyl ester hydrochloride as a white solid. LCMS MH+=475.

### Example 52

### (4-Chlorophenyl)-carbamic acid (S)-1-[(S)-3-(4-chlorophenoxy)-piperidin-1-ylmethyl]-2,2,2-trifluoroethyl ester hydrochloride (example 52a)

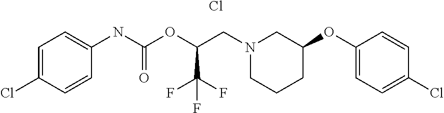 [SMILES: Cl ; O=C(Nc1ccc(Cl)cc1)O[C@@H](CN1CCC[C@H](Oc2ccc(Cl)cc2)C1)C(F)(F)F]

### and

### (4-Chlorophenyl)-carbamic acid (S)-1-[(R)-3-(4-chlorophenoxy)-piperidin-1-ylmethyl]-2,2,2-trifluoroethyl ester hydrochloride (example 52b)

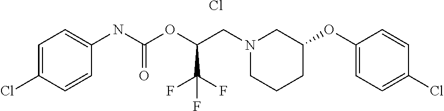 [SMILES: Cl ; O=C(Nc1ccc(Cl)cc1)O[C@@H](CN1CCC[C@@H](Oc2ccc(Cl)cc2)C1)C(F)(F)F]

The diastereomeric mixture of (4-chloro-phenyl)-carbamic acid (S)-1-[3-(4-chloro-phenoxy)-piperidin-1-ylmethyl]-2,2,2-trifluoroethyl ester, prepared in example 45, was separated by SFC chromatography (Diacel AD column, 3×25 cm, 20% methanol/CO₂ eluent) to provide 2 peaks which provided (4-chlorophenyl)-carbamic acid (S)-1-[(S)-3-(4-chlorophenoxy)-piperidin-1-ylmethyl]-2,2,2-trifluoroethyl ester and (4-chlorophenyl)-carbamic acid (S)-1-[(R)-3-(4-chlorophenoxy)-piperidin-1-ylmethyl]-2,2,2-trifluoroethyl ester, which were converted to the corresponding HCl salts to afford 30 mg of (4-chlorophenyl)-carbamic acid (S)-1-[(S)-3-(4-chlorophenoxy)-piperidin-1-ylmethyl]-2,2,2-trifluoroethyl ester hydrochloride as an off-white solid (LCMS MH+=477) and 30 mg of (4-chlorophenyl)-carbamic acid (S)-1-[(R)-3-(4-chlorophenoxy)-piperidin-1-ylmethyl]-2,2,2-trifluoroethyl ester hydrochloride as a white solid (LCMS MH+=477). Stereochemical assignments were based on analogy to examples 6 and 5 respectively.

### Example 53

(4-Chlorophenyl)-carbamic acid (S)-2,2,2-trifluoro-1-(3′,4′,5′,6′-tetrahydro-2′H-[2,3′]bipyridinyl-1′-ylmethyl)-ethyl ester hydrochloride

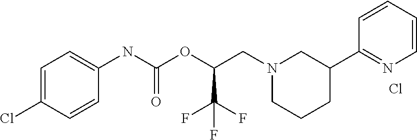 [SMILES: O=C(Nc1ccc(Cl)cc1)O[C@@H](CN1CCCC(c2ccccn2)C1)C(F)(F)F ; Cl]

Prepared by a similar procedure to example 39 except substituting 1′,2′,3′,4′,5′,6′-hexahydro-[2,3′]bipyridinyl for phenyl-(piperidin-3-yl)methanone afforded 110 mg of (4-chlorophenyl)-carbamic acid (S)-2,2,2-trifluoro-1-(3′,4′,5′,6′-tetrahydro-2′H-[2,3]bipyridinyl-1′-ylmethyl)-ethyl ester hydrochloride as a white solid. LCMS MH+=428.

### Example 54

### (4-Chlorophenyl)-carbamic acid (S)-1-[3-(4-cyanopyridin-2-yloxy)-piperidin-1-ylmethyl]-2,2,2-trifluoroethyl ester hydrochloride

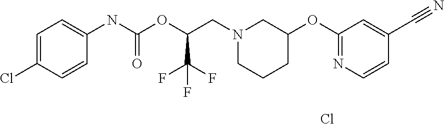 [SMILES: N#Cc1ccnc(OC2CCCN(C[C@H](OC(=O)Nc3ccc(Cl)cc3)C(F)(F)F)C2)c1 ; Cl]

In a 5 mL round-bottomed flask, tert-butyl 3-(4-cyanopyridin-2-yloxy)piperidine-1-carboxylate (200 mg, 659 μmol) was combined with dichloromethane (1 ml) to give a colorless solution. TFA (752 mg, 508 μl, 6.59 mmol) was added and the reaction mixture was stirred at room temperature for 2 h. The mixture was concentrated in vacuo and treated with triethylamine (0.3 ml). The resulting 2-(piperidin-3-yloxy)-isonicotinonitrile (130 mg, 640 μmol) was combined with acetonitrile (10.0 ml) to give a colorless solution. (S)-2-(Trifluoromethyl)oxirane (143 mg, 111 μl, 1.28 mmol, Eq: 2) was added and the reaction mixture was stirred at room temperature for 2 h. The crude reaction mixture was concentrated in vacuo to afford 190 mg (94%) of 2-(1-((S)-3,3,3-trifluoro-2-hydroxypropyl)piperidin-3-yloxy)isonicotinonitrile as an oil and used as is in the next reaction.

In a 10 mL round-bottomed flask, 2-(1-((S)-3,3,3-trifluoro-2-hydroxypropyl)piperidin-3-yloxy)isonicotinonitrile (190 mg, 603 μmol) was combined with acetonitrile (2.00 ml) to give a colorless solution. 1-Chloro-4-isocyanatobenzene (97.2 mg, 633 μmol) was added and the resultant mixture was stirred at room temperature overnight. The crude reaction mixture was concentrated in vacuo and purified by flash chromatography (silica, hexane to 60% ethyl acetate/hexane) to give an oil (300 mg). This oil was dissolved in ether and hexane. A solution of 4N HCl (0.2 ml) was added and the mixture was concentrated to afford 136 mg (45%) of (4-chloro-phenyl)-carbamic acid (S)-1-[3-(4-cyanopyridin-2-yloxy)-piperidin-1-ylmethyl]-2,2,2-trifluoroethyl ester hydrochloride as a white solid. LCMS MH+=469.

### Example 55

### (4-Chlorophenyl)-carbamic acid (S)-2,2,2-trifluoro-1-[3-(pyridin-3-ylmethoxy)-piperidin-1-ylmethyl]-ethyl ester hydrochloride

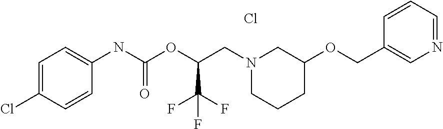 [SMILES: O=C(Nc1ccc(Cl)cc1)O[C@@H](CN1CCCC(OCc2cccnc2)C1)C(F)(F)F ; Cl]

Prepared by a similar procedure to example 39 except substituting 3-(piperidin-3-yloxymethyl)-pyridine for phenyl-(piperidin-3-yl)methanone afforded 185 mg of (4-chlorophenyl)-carbamic acid (S)-2,2,2-trifluoro-1-[3-(pyridin-3-ylmethoxy)-piperidin-1-ylmethyl]-ethyl ester hydrochloride as a yellow solid. LCMS MH+=458.

### Example 56

### (4-Chlorophenyl)-carbamic acid (S)-2,2,2-trifluoro-1-(6-trifluoromethyl-3′,4′,5′,6′-tetrahydro-2′H-[2,3′]bipyridinyl-1′-ylmethyl)-ethyl ester hydrochloride

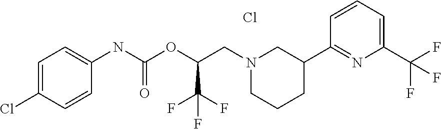 [SMILES: O=C(Nc1ccc(Cl)cc1)O[C@@H](CN1CCCC(c2cccc(C(F)(F)F)n2)C1)C(F)(F)F ; Cl]

Prepared by a similar procedure to example 39 except substituting 6-trifluoromethyl-1′,2′,3′,4′,5′,6′-hexahydro-[2,3′]bipyridine for phenyl-(piperidin-3-yl)methanone afforded 46 mg of (4-chlorophenyl)-carbamic acid (S)-2,2,2-trifluoro-1-(6-trifluoromethyl-3′,4′,5′,6′-tetrahydro-2′H-[2,3′]bipyridinyl-1′-ylmethyl)-ethyl ester hydrochloride as a white solid. LCMS MH+=496.

### Example 57

### (4-Chlorophenyl)-carbamic acid (S)-2,2,2-trifluoro-1-(6′-trifluoromethyl-3,4,5,6-tetrahydro-2H-[3,3′]bipyridinyl-1-ylmethyl)-ethyl ester hydrochloride

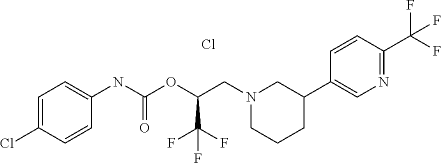 [SMILES: O=C(Nc1ccc(Cl)cc1)O[C@@H](CN1CCCC(c2ccc(C(F)(F)F)nc2)C1)C(F)(F)F ; Cl]

In a 2 mL vial, 5-(piperidin-3-yl)-2-(trifluoromethyl)pyridine (120 mg, 521 μmol) was combined with acetonitrile (1 mL) to give a colorless solution. (S)-2-(Trifluoromethyl)oxirane (292 mg, 2.61 mmol) was added and the reaction mixture was stirred at room temperature overnight and then concentrated in vacuo to give 160 mg of (2S)-1,1,1-trifluoro-3-(3-(6-(trifluoromethyl)pyridin-3-yl)piperidin-1-yl)propan-2-ol as a viscous oil. LCMS MH+=343.

In a 10 mL pear-shaped flask, (2S)-1,1,1-trifluoro-3-(3-(6-(trifluoromethyl)pyridin-3-yl)piperidin-1-yl)propan-2-ol (146 mg, 427 μmol) and 1-chloro-4-isocyanatobenzene (65.5 mg, 427 μmol) were combined with acetonitrile (5 ml) to give a colorless solution. The reaction mixture was stirred at room temperature for 2 h and then concentrated in vacuo. The resulting residue was purified by column chromatography (dichloromethane to 40% ethyl acetate/dichloromethane eluent) to afford a waxy solid. This solid was dissolved in ether/hexane, treated with a solution of 4N HCl in dioxane (0.2 ml) and then concentrated to afford 180 (79%) of (4-chlorophenyl)-carbamic acid (S)-2,2,2-trifluoro-1-(6′-trifluoromethyl-3,4,5,6-tetrahydro-2H-[3,3′]bipyridinyl-1-ylmethyl)-ethyl ester hydrochloride as a white solid. LCMS MH+=496.

### Example 58

### (5-Chloropyridin-2-yl)-carbamic acid (S)-2,2,2-trifluoro-1-[(R)-3-(3-trifluoromethyl-phenyl)-piperidin-1-ylmethyl]-ethyl ester hydrochloride

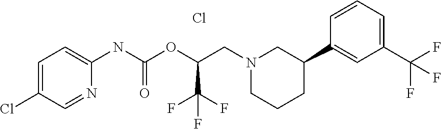 [SMILES: O=C(Nc1ccc(Cl)cn1)O[C@@H](CN1CCC[C@H](c2cccc(C(F)(F)F)c2)C1)C(F)(F)F ; Cl]

5-Chloropicolinic acid (82 mg, 516 μmol), (2S)-1,1,1-trifluoro-3-(3-(3-(trifluoromethyl)phenyl)-piperidin-1-yl)propan-2-ol (176 mg, 516 μmol), DPPA (156 mg, 567 μmol) and TEA (0.1 mL, 712 μmol) were combined in a reaction tube containing toluene (2.5 mL). The clear solution was stirred at room temperature for 30 minutes and then put into an oil bath preheated to 80° C. The mixture was stirred at 80° C. for 3 hrs. Solvents were evaporated and the residue was extracted with ethyl acetate (containing 50% hexanes) and water. The organic layer was washed sodium bicarbonate solution, dried and concentrated. The residue was purified through ISCO flash column chromatography (0% to 20% ethyl acetate in hexanes, 12 g silica gel). The first UV active fraction was pooled and concentrated. The oily residue was dissolved into ether and treated with 0.2 mL of 1N HCl in ether. Solvents were evaporated and the residue was treated with dry ether. The white solid was filtered to give 36 mg (13%) of (5-chloropyridin-2-yl)-carbamic acid (S)-2,2,2-trifluoro-1-[(R)-3-(3-trifluoromethylphenyl)-piperidin-1-ylmethyl]-ethyl ester hydrochloride as a white solid. LCMS MH+=496.

### Example 59

### (5-Chloropyridin-2-yl)-carbamic acid (S)-2,2,2-trifluoro-1-[3-(3-trifluoromethylphenyl)-piperidin-1-ylmethyl]-ethyl ester hydrochloride

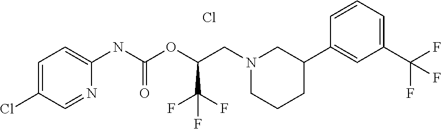 [SMILES: O=C(Nc1ccc(Cl)cn1)O[C@@H](CN1CCCC(c2cccc(C(F)(F)F)c2)C1)C(F)(F)F ; Cl]

(5-chloropyridin-2-yl)-carbamic acid (S)-2,2,2-trifluoro-1-[3-(3-trifluoromethyl-phenyl)-piperidin-1-ylmethyl]-ethyl ester hydrochloride (18 mg, 6%) was prepared according to the methods described for Example 58 except that the mixed fraction containing both diastereomers were combined from the flash chromatography. LCMS MH+=496.

### Example 60

### (4-Bromophenyl)-carbamic acid (S)-2,2,2-trifluoro-1-[3-(3-methoxyphenyl)-piperidin-1-ylmethyl]-ethyl ester hydrochloride

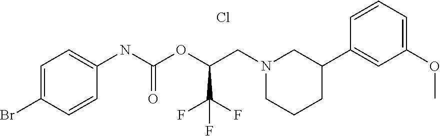 [SMILES: COc1cccc(C2CCCN(C[C@H](OC(=O)Nc3ccc(Br)cc3)C(F)(F)F)C2)c1 ; Cl]

To a solution of 3-(3-methoxyphenyl)piperidine hydrochloride (500 mg, 2.2 mmol) and DIPEA (1.15 mL, 6.59 mmol) in acetonitrile was added (S)-2-trifluoromethyloxirane (298 mg, 2.63 mmol) at room temperature. The mixture was stirred at room temperature over the weekend. The solvent was removed by evaporation to dryness to afford 666 mg (99%) of (S)-1,1,1-trifluoro-3-[3-(3-methoxyphenyl)-piperidin-1-yl]-propan-2-ol as an oil, which was used directly for next step.

(S)-1,1,1-Trifluoro-3-[3-(3-methoxyphenyl)-piperidin-1-yl]-propan-2-ol (41 mg, 135 μmole) and 1-bromo-4-isocyanatobenzene (26.7 mg, 135 μmol) were combined in acetonitrile (2 mL). The reaction mixture was stirred at room temperature for 24 h. The solution was separated by reverse-phase column chromatography (50-100% acetonitrile in water). The desired fractions were collected, diluted with more water, treated with 5 drops of concentrated HCl, and lyophilized to give 38 mg (54%) of (4-bromophenyl)-carbamic acid (S)-2,2,2-trifluoro-1-[3-(3-methoxyphenyl)-piperidin-1-ylmethyl]-ethyl ester hydrochloride as a white solid. LCMS MH+=500, 502.

### Example 61

### (4-Fluorophenyl)-carbamic acid (S)-2,2,2-trifluoro-1-[3-(3-methoxyphenyl)-piperidin-1-ylmethyl]-ethyl ester hydrochloride

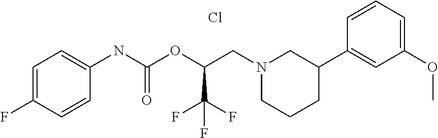 [SMILES: COc1cccc(C2CCCN(C[C@H](OC(=O)Nc3ccc(F)cc3)C(F)(F)F)C2)c1 ; Cl]

Prepared by a similar procedure to example 60 except substituting 1-fluoro-4-isocyanatobenzene for 1-bromo-4-isocyanatobenzene afforded 18 mg (28%) of (4-fluorophenyl)-carbamic acid (S)-2,2,2-trifluoro-1-[3-(3-methoxyphenyl)-piperidin-1-ylmethyl]-ethyl ester hydrochloride as a white solid. LCMS MH+=441.

### Example 62

### (4-Chlorophenyl)-carbamic acid (S)-2,2,2-trifluoro-1-[3-(3-methanesulfonylphenyl)-piperidin-1-ylmethyl]-ethyl ester hydrochloride

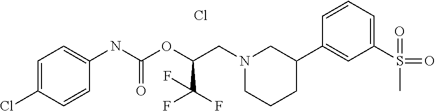 [SMILES: CS(=O)(=O)c1cccc(C2CCCN(C[C@H](OC(=O)Nc3ccc(Cl)cc3)C(F)(F)F)C2)c1 ; Cl]

### Step 1

To a mixture of 3-bromopyridine (2.53 g, 16 mmol), 3-(methylsulfonyl)phenylboronic acid (3.2 g, 16 mmol), cesium carbonate (10.4 g, 32 mmol), DMF (60 mL), t-butanol (5 mL) and water (15 mL) was bubbled nitrogen gas for 1 hour at room temperature, followed by addition of PdCl₂(DPPF)-dichloromethane adduct (585 mg, 0.8 mmol). The reaction vessel was then sealed and heated to 88° C. for 12 h. The mixture was cooled to room temperature and filtered through Celite. The filtrate was evaporated to dryness under reduced pressure. The residue was stirred with ethyl acetate (300 mL) overnight, and then filtered. The filtrate was evaporated and the residue was purified by flash chromatography (silica gel, 0-5% methanol in dichloromethane) to afford 3.85 g (100%) of 3-(3-methanesulfonylphenyl)-pyridine as an oily pure product. LCMS MH+=234.

### Step 2

A mixture of 3-(3-methanesulfonylphenyl)-pyridine (3.85 g, 16.5 mmol), platinum (IV) oxide hydrate (404 mg, 1.65 mmol) and concentrated HCl (10 mL) in methanol (40 mL) and water (8 ml) were subjected to hydrogenation at room temperature and 50 psi on a Parr Shaker for 12 h. The mixture was then filtered and the filtrate was hydrogenated under same procedure two more times. The mixture was then filtered and filtrate was made basic with 10% sodium carbonate and extracted with ethyl acetate (3×). The combined ethyl acetate phase was washed with brine, dried over sodium sulfate, filtered, and evaporated to give 777 mg (20%) of 3-(3-methanesulfonylphenyl)piperidine, which was used as is for the next step. LCMS MH+=240.

### Step 3

3-(3-Methanesulfonylphenyl)piperidine (215 mg, 898 μmol) and (S)-2-trifluoromethyloxirane (151 mg, 1.35 mmol) were combined in acetonitrile (3 mL). The resultant reaction mixture was stirred at room temperature for 12 h and concentrated in vacuo to afford 321 mg (100%) of (S)-1,1,1-trifluoro-3-[3-(3-methanesulfonylphenyl)-piperidin-1-yl]-propan-2-ol, which was used as is in the next step. LCMS MH+=352.

### Step 4

(S)-1,1,1-trifluoro-3-[3-(3-methanesulfonylphenyl)-piperidin-1-yl]-propan-2-ol (50 mg, 142 μmole) and 1-chloro-4-isocyanatobenzene (21.9 mg, 142 μmol) were combined in acetonitrile (2 mL). The reaction mixture was stirred at room temperature for 3 days. To the reaction mixture was added several drops of DMSO to make a clear solution. The solution was separated by reverse-phase column chromatography (50-100% acetonitrile in water). The desired fractions were collected, diluted with more water, treated with 5 drops of concentrated HCl, and lyophilized to give 51 mg (66%) of (4-chlorophenyl)-carbamic acid (S)-2,2,2-trifluoro-1-[3-(3-methanesulfonylphenyl)-piperidin-1-ylmethyl]-ethyl ester hydrochloride as a white solid. LCMS MH+=505.

### Example 63

### (4-Fluorophenyl)-carbamic acid (S)-2,2,2-trifluoro-1-[3-(3-methanesulfonylphenyl)-piperidin-1-ylmethyl]-ethyl ester hydrochloride

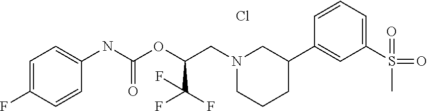 [SMILES: CS(=O)(=O)c1cccc(C2CCCN(C[C@H](OC(=O)Nc3ccc(F)cc3)C(F)(F)F)C2)c1 ; Cl]

Prepared by a similar procedure to example 62 except substituting 1-fluoro-4-isocyanatobenzene for 1-chloro-4-isocyanatobenzene afforded 57 mg (76%) of (4-fluorophenyl)-carbamic acid (S)-2,2,2-trifluoro-1-[3-(3-methanesulfonylphenyl)-piperidin-1-ylmethyl]-ethyl ester hydrochloride as a white solid. LCMS MH+=489.

### Example 64

### (4-Chlorophenyl)-carbamic acid (S)-1-(3-benzylpyrrolidin-1-ylmethyl)-2,2,2-trifluoro-ethyl ester

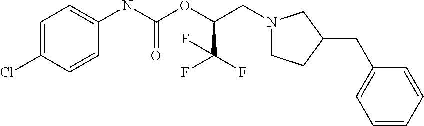 [SMILES: O=C(Nc1ccc(Cl)cc1)O[C@@H](CN1CCC(Cc2ccccc2)C1)C(F)(F)F]

To a solution of (S)-2-(trifluoromethyl)oxirane (50 mg, 446 μmole) in acetonitrile (1 mL) at room temperature was added a solution of 3-benzylpyrrolidine (72 mg, 446 μmole) in acetonitrile (1 mL). The solution was stirred at room temperature for 5 h followed by evaporation in vacuo to afford (2S)-3-(3-benzylpyrrolidin-1-yl)-1,1,1-trifluoropropan-2-ol that was used directly for next step.

(2S)-3-(3-benzylpyrrolidin-1-yl)-1,1,1-trifluoropropan-2-ol (122 mg, 446 μmole) and 1-chloro-4-isocyanatobenzene (68.5 mg, 446 μmol) were combined in acetonitrile (2 mL). The reaction mixture was stirred at room temperature for 24 h. To the reaction mixture was added several drops of DMSO to make a clear solution. The solution was purified by reverse-phase column chromatography (35-100% acetonitrile in water) to give 117 mg (60%) of (4-chlorophenyl)-carbamic acid (S)-1-(3-benzylpyrrolidin-1-ylmethyl)-2,2,2-trifluoro-ethyl ester as an off-white solid after lyophilzation, 115 mg (60%) of an off-white solid. MH+=427.

### Example 65

### (3-Chloro-4-fluoro-phenyl)-carbamic acid (S)-2,2,2-trifluoro-1-[3-(3-trifluoromethyl-phenyl)-piperidin-1-ylmethyl]-ethyl ester

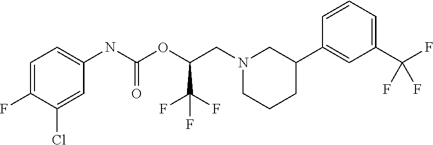 [SMILES: O=C(Nc1ccc(F)c(Cl)c1)O[C@@H](CN1CCCC(c2cccc(C(F)(F)F)c2)C1)C(F)(F)F]

In a 50 mL round bottle, 2-chloro-1-fluoro-4-isocyanatobenzene (20 mg, 117 μmol) was combined with dichloromethane (93.3 ml) to give a colorless solution. (S)-1,1,1-Trifluoro-3-[3-(3-trifluoromethylphenyl)-piperidin-1-yl]-propan-2-ol (40.0 mg, 117 μmol) was added. The reaction was stirred at room temperature for 1 hour. The product was extracted with dichloromethane, dried and purified by column chromatography (hexanes/EtOAc=90/10 eluant) to afford 27 mg (45%) of (3-chloro-4-fluoro-phenyl)-carbamic acid (S)-2,2,2-trifluoro-1-[3-(3-trifluoromethylphenyl)-piperidin-1-ylmethyl]-ethyl ester. LCMS MH+=513.2.

### Example 66

### (3,4-Dichlorophenyl)-carbamic acid(S)-2,2,2-trifluoro-1-[3-(3-trifluoromethyl-phenyl)piperidin-1-ylmethyl]-ethyl ester

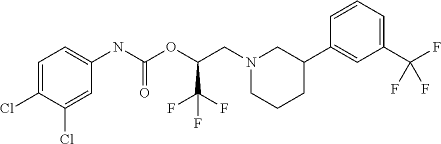 [SMILES: O=C(Nc1ccc(Cl)c(Cl)c1)O[C@@H](CN1CCCC(c2cccc(C(F)(F)F)c2)C1)C(F)(F)F]

Prepared by a similar procedure to example 65 except substituting 1,2-dichloro-4-isocyanatobenzene for 2-chloro-1-fluoro-4-isocyanatobenzene afforded 33 mg (58%) of (3,4-dichlorophenyl)-carbamic acid (S)-2,2,2-trifluoro-1-[3-(3-trifluoromethylphenyl)piperidin-1-ylmethyl]-ethyl ester LCMS MH+=529.

### Example 67

### (3,4-Difluorophenyl)-carbamic acid (S)-2,2,2-trifluoro-1-[3-(3-trifluoromethylphenyl)piperidin-1-ylmethyl]-ethyl ester

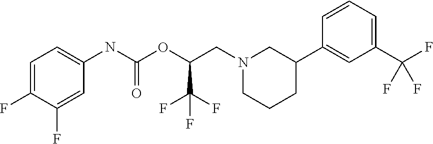 [SMILES: O=C(Nc1ccc(F)c(F)c1)O[C@@H](CN1CCCC(c2cccc(C(F)(F)F)c2)C1)C(F)(F)F]

Prepared by a similar procedure to example 65 except substituting 1,2-difluoro-4-isocyanatobenzene for 2-chloro-1-fluoro-4-isocyanatobenzene afforded 50 mg (53%) of (3,4-difluorophenyl)-carbamic acid (S)-2,2,2-trifluoro-1-[3-(3-trifluoromethylphenyl)piperidin-1-ylmethyl]-ethyl ester. LCMS MH+=497.

### Example 68

### (6-Chloropyridin-3-yl)-carbamic acid (S)-2,2,2-trifluoro-1-[3-(3-trifluoromethylphenyl)-piperidin-1-ylmethyl]-ethyl ester

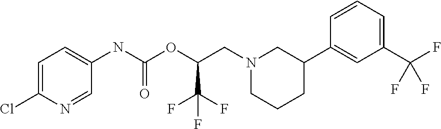 [SMILES: O=C(Nc1ccc(Cl)nc1)O[C@@H](CN1CCCC(c2cccc(C(F)(F)F)c2)C1)C(F)(F)F]

In a 50 mL round bottle, 2-chloro-5-isocyanatopyridine (30 mg, 194 μmol) was combined with acetonitrile (5 ml) to give a colorless solution. (S)-1,1,1-Trifluoro-3-[3-(3-trifluoromethylphenyl)-piperidin-1-yl]-propan-2-ol (66.6 mg, 194 μmol) was added. The reaction was stirred at room temperature for 1 hour. The product was extracted with dichloromethane, dried and purified by column chromatography (hexanes/EtOAc=90/10 eluant) to afford 20 mg (21%) of (6-chloropyridin-3-yl)-carbamic acid (S)-2,2,2-trifluoro-1-[3-(3-trifluoromethylphenyl)-piperidin-1-ylmethyl]-ethyl ester. LCMS MH+=496.1.

### Example 69

### IC₅₀ Determination of Exemplified Compounds Dose Response Assay: ChanTest hTRPA1-CHO Stably Transfected Cell Line

Cell Culture and Assay Reagents:

| | |
|---|---|
| Ham's F12 | (GIBCO #11765-047) |
| Tetracycline-free Fetal Bovine Serum | (ClonTech#631106, Lot |
| | A301097018) |
| Blasticidin (10 mg/ml stock) | (GIBCO #A11139-02) |
| Zeocin (100 mg/ml stock) | (GIBCO #R250-01) |
| Doxycycline | (SIGMA #D9891) |
| Penicillin-Spreptomycin solution (100X) | (GIBCO #15140-122) |
| GlutaMAX (100X) | (GIBCO #35050) |
| Trypsin-EDTA | (GIBCO #25200-056) |
| PBS (without Calcium and Magnesium) | (GIBCO #14190) |
| HBSS | (GIBCO #14025) |
| Hepes | (GIBCO #15630) |
| BSA (fatty acid free, low endotoxin) | (SIGMA #A8806-5G) |
| DMSO | (SIGMA #D2650) |
| AP-18 | (SIGMA #A7232) |
| Cinnamaldehyde | (SIGMA #W228613) |
| ATP | (SIGMA #A-6419) |
| 2-Aminoethyl diphenylborinate | (SIGMA #D9754) |
| Menthol | (Sigma #M2772) |
| FLIPR Calcium 3 Assay Kit | (Molecular Devices #R8108) |
| Probenecid | (INVITROGEN #36400) |
| Plates | (BD #35-3962) |

### CHO-K1 Tet-On_HOMSA_TRPA1_Clone_{—}20

Chinese Hamster Ovary cells, inducible expression
Clone #20, received at passage #26 Channel expression in this cell line has been shown to be stable for at least 80 passages
Verified Mycoplasma free with MycoAlert Kit
Cell line expanded and banked

### Growth Conditions:

Growth Media for CHO-K1 Tet-On_HOMSA_TRPA1_Clone_{—}20
Ham's F-12 with 10% tetracycline-free FBS
1× penicillin-streptomycin
1× glutamax
0.01 mg/ml Blasticidin
0.40 mg/ml Zeocin
The cell line doubling rate was ˜15 hours. The culture plates did not exceed 80% confluency.
To induce expression, tetracycline was added to blasticidin/zeocin-free media at a final concentration of 1 ug/ml. Experiments were run at 24 hours post induction.

Plating Conditions CHOK1/TRPA1 Cells:
Harvested cells with 0.025% trypsin/EDTA.
Resuspended cells in growth media without selection antibiotics.
Measured cell density and diluted to 2.4×10⁵ cells/ml in media containing 1 ug/ml Doxycycline Plate 25 ul/well into 384 well black/clear tissue culture-treated plates.
Incubated overnight at 37° C.

### Calcium Flux Assay:

### Day of Assay:

Reagents:
Replacement Buffer: Hank's Balanced Salt Solution, 20 mM HEPES along with 0.005% BSA and 2× Probenecid
Dye Loading Buffer: Cal-3 NW Calcium dye was prepared by dissolving the contents of one vial with 500 ml Hank's Balanced Salt Solution containing 20 mM HEPES.
Control compounds for CHOK1/TRPA1 cells:
   AP-18, stock 10 mM, prepare 3.5× compound dilution in a Compound Buffer (HBSS/20 mM HEPES/0.005% BSA)—final concentration 10 uM.

Preparation of Cinnamaldehyde (Agonist Addition):

### FW=132.16

Specific gravity=1.046 gm/cc
1.32 gm/1.046 gm/cc=1.26 ml of stock
Add 1.74 ml DMSO=3.3 M stock
Working solution 4.5× (final 100 uM in Compound Buffer: HBSS/20 mM HEPES/0.005% BSA)

Compounds dilutions were prepared from 5 or 10 mM stock (100% DMSO):
Adjustments of volumes and concentrations were made at time of titration to reflect desired final assay concentrations.
Compounds were tested at either 20 μM three folds dilution 11 steps out or 30 μM two folds dilution 11 steps out.
3 μl of diluted compound were transferred into Weidmann 384-well plate in duplicates side-by-side.

Compound plates were resuspended with 100 ul of HBSS/20 mM HEPES/0.005% BSA buffer (Compound Buffer):

column 1A-H: buffer/DMSO (bk)

column 2A-H: AP-18 (control antagonist for CHOK1 TRPA1 cells)

column 1I-P: ATP (control for CHOK1 teton cells)

column 2 I-P: 2APB (control antagonist for CHOK1/TRPM8 cells).

Growth media was removed from the cell plates (20 ul) and 20 ul of the Replacement Buffer was added followed by addition of 25 ul of diluted dye. All three steps were performed using a Plate Washer BioTek 407. The plates were then incubated for 30′ at RT.

After incubation, both the cell and compound plates were brought to the FLIPR and 20 ul of the diluted compounds/antagonist/bk were transferred to the cell plates by the FLIPR. Plates were then incubated for 30′ at room temperature. After 30′ incubation, plates were returned to the FLIPR and 20 ul of 4.5× Cinnamaldehyde was added to the cell plates. During the compound addition as well as agonist addition, fluorescence readings were taken simultaneously from all 384 wells of the cell plate every 1.5 seconds. Five readings were taken to establish a stable baseline, then 20 ul of sample was rapidly (30 ul/sec) and simultaneously added to each well of the cell plate. The fluorescence was continuously monitored before, during and after sample/agonist addition for a total elapsed time of 100 seconds (compound addition) and 120 seconds (agonist addition). Responses (increase in peak fluorescence) in each well following agonist addition was determined. The initial fluorescence reading from each well, prior to ligand stimulation, was used a zero baseline value for the data from that well. The responses were expressed as % inhibition of the inhibitor control as shown in Table 1 below:

TABLE 1

| Example | hTRPA1: |
|---|---|
| No. | IC50 μM |
| 1 | 4.971 |
| 2 | 0.345 |
| 3 | 0.1215 |
| 4 | 7.7 |
| 5 | 0.333 |
| 6 | 1.496 |
| 7 | 6.4 |
| 8 | 6.2 |
| 9 | 0.339 |
| 10 | 1.336 |
| 11 | 0.08715 |
| 12 | 0.55 |
| 13 | 0.277 |
| 14 | 0.137 |
| 15 | 0.5635 |
| 16 | 4.1625 |
| 17 | 0.8995 |
| 18 | 0.8835 |
| 19 | 0.2885 |
| 20 | 0.165 |
| 21 | 0.203 |
| 22a | 4.147 |
| 22b | 1.7645 |
| 23 | 0.02153 |
| 24 | 0.3115 |
| 25 | 0.135 |
| 26 | 1.401 |
| 27 | 2.26 |
| 28 | 1.8535 |
| 29 | 0.03537 |
| 30 | 0.0104 |
| 31 | 0.1685 |
| 32 | 0.1128 |
| 33 | 0.0783 |
| 34 | 0.33925 |
| 35 | 0.3335 |
| 36 | 0.625 |
| 37 | 0.5217 |
| 38 | 0.622 |
| 39 | 0.865 |
| 40 | 0.1905 |
| 41 | 0.32 |
| 42 | 0.4435 |
| 43 | 0.298 |
| 44 | 0.172 |
| 45 | 0.139 |
| 46 | 0.411 |
| 47 | 0.2195 |
| 48 | 0.14725 |
| 49 | 0.40825 |
| 50 | 0.05513 |
| 51 | 0.1775 |
| 52a | 0.114 |
| 52b | 0.961 |
| 53 | 1.924 |
| 54 | 0.596 |
| 55 | 0.803 |
| 56 | 0.246 |
| 57 | 0.342 |
| 58 | 0.174 |
| 59 | 0.27 |
| 60 | 0.365 |
| 61 | 0.2725 |
| 62 | 1.1625 |
| 63 | 2.914 |
| 64 | 4.992 |
| 65 | 0.257 |
| 66 | 0.2 |
| 67 | 0.1765 |
| 68 | 0.1915 |

It is to be understood that the invention is not limited to the particular embodiments of the invention described above, as variations of the particular embodiments may be made and still fall within the scope of the appended claims.

1. A compound of formula (I): 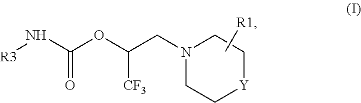
wherein:
Y is —(CH₂)ₙ— or —CF₂—;
n is 0, 1 or 2;
R1 is —X-R2, hydrogen, —CN, —CF₃, alkoxy, cycloalkyl, unsubstituted lower alkyl or lower alkyl substituted with alkoxy;
X is a single bond, —CH₂—, —O—, —C(O)—, S, —CH₂—O— or —O—CH₂—;
R2 is unsubstituted phenyl, phenyl mono- or bi-substituted independently with alkoxy, —CN, —CF₃, —OCF₃, halogen, —O(CH₂)₂OCH₃ or —SO₂CH₃, unsubstituted pyridinyl, pyridinyl substituted with —CN or —CF₃, or methyl-[1,2,4]oxadiaolyl; and
R3 is unsubstituted phenyl, unsubstituted pyridinyl, phenyl mono- or bi-substituted independently with halogen, or pyridinyl mono or bi-substituted independently with halogen,
or a pharmaceutically acceptable salt thereof,
with the proviso that said compound is not 1-piperidineethanol-a-(trifluoromethyl)-phenylcarbamate ester, carbamic acid (4-chlorophenyl)-2,2,2-trifluoro-1-(1-piperidinylmethyl)ethyl ester, carbamic acid (3-fluorophenyl)-2,2,2-trifluoro-1-(1-piperidinylmethyl)ethyl ester or carbamic acid (4-methoxyphenyl)-2,2,2-trifluoro-1-(1-piperidinylmethyl)ethyl ester.

2. The compound according to claim 1, wherein Y is —(CH₂)ₙ—.

3. The compound according to claim 1, wherein n is 1.

4. The compound according to claim 1, wherein R1 is —X-R2.

5. The compound according to claim 1, wherein R1 is —CN, —CF₃, alkoxy, cycloalkyl, unsubstituted lower alkyl or lower alkyl substituted with alkoxy.

6. The compound according to claim 1, wherein X is a single bond.

7. The compound according to claim 1, wherein X is —CH₂—.

8. The compound according to claim 1, wherein X is —O—.

9. The compound according to claim 1, wherein R2 is unsubstituted phenyl.

10. The compound according to claim 1, wherein R2 is phenyl mono- or bi-substituted independently with alkoxy, —CN, —CF₃, —OCF₃, halogen, —O(CH₂)₂OCH₃ or —SO₂CH₃.

11. The compound according to claim 1, wherein R2 is unsubstituted pyridinyl.

12. The compound according to claim 1, wherein R2 is pyridinyl substituted with —CN or —CF₃.

13. The compound according to claim 1, wherein R3 is phenyl mono- or bi-substituted independently with halogen.

14. The compound according to claim 1, wherein R3 is pyridinyl mono- or bi-substituted independently with halogen.

15. The compound according to claim 1, wherein said compound is:
(4-chlorophenyl)-carbamic acid 2,2,2-trifluoro-1-[3-(2-methoxyphenyl)-piperidin-1-ylmethyl]-ethyl ester;
(4-Chlorophenyl)-carbamic acid 2,2,2-trifluoro-1-[3-(3-methoxyphenyl)-piperidin-1-ylmethyl]-ethyl ester hydrochloride;
(4-Chlorophenyl)-carbamic acid (S)-2,2,2-trifluoro-1-[(R)-3-(3-methoxyphenyl)-piperidin-1-ylmethyl]-ethyl ester hydrochloride;
(4-Chlorophenyl)-carbamic acid (S)-2,2,2-trifluoro-1-[(S)-3-(3-methoxyphenyl)-piperidin-1-ylmethyl]-ethyl ester hydrochloride;
(4-Chlorophenyl)-carbamic acid (S)-2,2,2-trifluoro-1-((R)-3-phenylpiperidin-1-ylmethyl)-ethyl ester hydrochloride;
(4-Chlorophenyl)-carbamic acid (S)-2,2,2-trifluoro-1-((S)-3-phenylpiperidin-1-ylmethyl)-ethyl ester hydrochloride;
(4-Chlorophenyl)-carbamic acid (R)-2,2,2-trifluoro-1-((R)-3-phenylpiperidin-1-ylmethyl)-ethyl ester hydrochloride;
(4-Chlorophenyl)-carbamic acid (R)-2,2,2-trifluoro-1-((S)-3-phenylpiperidin-1-ylmethyl)-ethyl ester hydrochloride;
(4-chlorophenyl)-carbamic acid 1-(3-benzylpiperidin-1-ylmethyl)-2,2,2-trifluoro-ethyl ester hydrochloride salt (1:1);
(4-Chlorophenyl)-carbamic acid (S)-1-((S)-3-benzylpiperidin-1-ylmethyl)-2,2,2-trifluoroethyl ester toluenesulfonic acid salt;
(4-Chlorophenyl)-carbamic acid (S)-1-((R)-3-benzylpiperidin-1-ylmethyl)-2,2,2-trifluoroethyl ester toluenesulfonic acid salt;
(4-chlorophenyl)-carbamic acid (S)-2,2,2-trifluoro-1-[3-(3-methoxybenzyl)-piperidin-1-ylmethyl]-ethyl ester hydrochloride salt (1:1);
(4-chlorophenyl)-carbamic acid 1-(3-cyanopiperidin-1-ylmethyl)-2,2,2-trifluoroethyl ester;
(4-Chlorophenyl)-carbamic acid 1-(3-benzyloxypiperidin-1-ylmethyl)-2,2,2-trifluoroethyl ester;
(4-chlorophenyl)-carbamic acid 2,2,2-trifluoro-1-(3-trifluoromethylpiperidin-1-ylmethyl)-ethyl ester;
(4-Chlorophenyl)-carbamic acid 2,2,2-trifluoro-1-(3-methoxypiperidin-1-ylmethyl)-ethyl ester;
(4-Chlorophenyl)-carbamic acid 1-(3-ethylpiperidin-1-ylmethyl)-2,2,2-trifluoroethyl ester;
(4-Chlorophenyl)-carbamic acid 1-(3-cyclohexylpiperidin-1-ylmethyl)-2,2,2-trifluoroethyl ester;
(4-Chlorophenyl)-carbamic acid 2,2,2-trifluoro-1-[3-(3-methoxypropyl)-piperidin-1-ylmethyl]-ethyl ester;
(4-Chlorophenyl)-carbamic acid 2,2,2-trifluoro-1-[3-(4-methoxybenzyl)-piperidin-1-ylmethyl]-ethyl ester;
(4-Chlorophenyl)-carbamic acid (S)-1-[3-(3,5-dimethoxyphenyl)-piperidin-1-ylmethyl]-2,2,2-trifluoroethyl ester;
(4-Chlorophenyl)-carbamic acid 2,2,2-trifluoro-1-[(S)-3-(pyridin-4-yloxy)-piperidin-1-ylmethyl]-ethyl ester;
(4-Chlorophenyl)-carbamic acid 2,2,2-trifluoro-1-[(R)-3-(pyridin-4-yloxy)-piperidin-1-ylmethyl]-ethyl ester;
(4-Chlorophenyl)-carbamic acid (S)-1-[(R)-3-(3,5-bis-trifluoromethylphenyl)-piperidin-1ylmethyl]-2,2,2-trifluoroethyl ester;
(4-Chlorophenyl)-carbamic acid (S)-1-[(S)-3-(3,5-bis-trifluoromethyl-phenyl)-piperidin-1ylmethyl]-2,2,2-trifluoroethyl ester;
(S)—N-(4-Chlorophenyl)-4,4,4-trifluoro-3-[(R)-3-(3-trifluoromethylphenyl)-piperidin-1-ylmethyl]-butyramide;
(S)—N-(4-Chlorophenyl)-4,4,4-trifluoro-3-[(S)-3-(3-trifluoromethylphenyl)-piperidin-1-ylmethyl]-butyramide;
(4-Chlorophenyl)-carbamic acid 2,2,2-trifluoro-1-(4-phenyl-azepan-1-ylmethyl)-ethyl ester;
(4-Chlorophenyl)-carbamic acid (S)-2,2,2-trifluoro-1-[3-(3-methyl-[1,2,4]oxadiazol-5-yl)-piperidin-1-ylmethyl]-ethyl ester hydrochloride;
(4-Chlorophenyl)-carbamic acid (S)-2,2,2-trifluoro-1-[3-(3-trifluoromethylphenyl)-piperidin-1-ylmethyl]-ethyl ester hydrochloride;
(4-Chlorophenyl)-carbamic acid (S)-2,2,2-trifluoro-1-[(R)-3-(3-trifluoromethylphenyl)-piperidin-1-ylmethyl]-ethyl ester hydrochloride;
(4-Chlorophenyl)-carbamic acid (S)-2,2,2-trifluoro-1-[3-(4-fluoro-3-methoxyphenyl)-piperidin-1-ylmethyl]-ethyl ester hydrochloride;
(4-Chlorophenyl)-carbamic acid (S)-2,2,2-trifluoro-1-[(R)-3-(4-fluoro-3-methoxyphenyl)-piperidin-1-ylmethyl]-ethyl ester hydrochloride;
(4-Chlorophenyl)-carbamic acid (S)-1-(4,4-difluoropiperidin-1-ylmethyl)-2,2,2-trifluoroethyl ester hydrochloride;
(4-Chlorophenyl)-carbamic acid (S)-2,2,2-trifluoro-1-{3-[4-fluoro-3-(2-methoxyethoxy)-phenyl]-piperidin-1-ylmethyl}-ethyl ester;
(4-Chlorophenyl)-carbamic acid (S)-2,2,2-trifluoro-1-{(R)-3-[4-fluoro-3-(2-methoxyethoxy)-phenyl]-piperidin-1-ylmethyl}-ethyl ester hydrochloride;
(4-Chlorophenyl)-carbamic acid 2,2,2-trifluoro-1-(3-phenylpiperidin-1-ylmethyl)-ethyl ester hydrochloride;
(4-Chlorophenyl)-carbamic acid 1-[3-(4-chlorophenyl)-piperidin-1-ylmethyl]-2,2,2-trifluoro ethyl ester;
(4-Chlorophenyl)-carbamic acid (S)-2,2,2-trifluoro-1-[3-(4-methoxyphenyl)-piperidin-1-ylmethyl]-ethyl ester;
(4-Chlorophenyl)-carbamic acid (S)-1-(3-benzoylpiperidin-1-ylmethyl)-2,2,2-trifluoroethyl ester hydrochloride;
(4-Chlorophenyl)-carbamic acid (S)-2,2,2-trifluoro-1-(3-phenoxypiperidin-1-ylmethyl)-ethyl ester hydrochloride;
(4-Chlorophenyl)-carbamic acid (S)-2,2,2-trifluoro-1-[3-(3-fluorophenyl)-piperidin-1-ylmethyl]-ethyl ester hydrochloride;
(4-Chlorophenyl)-carbamic acid (S)-2,2,2-trifluoro-1-[3-(4-fluorophenyl)-piperidin-1-ylmethyl]-ethyl ester hydrochloride;
(4-Chlorophenyl)-carbamic acid (S)-1-[3-(3-ethoxyphenyl)-piperidin-1-ylmethyl]-2,2,2-trifluoroethyl ester hydrochloride;
(4-Chlorophenyl)-carbamic acid (S)-2,2,2-trifluoro-1-(3-phenylsulfanylpiperidin-1-ylmethyl)-ethyl ester hydrochloride;
(4-Chlorophenyl)-carbamic acid (S)-1-[3-(4-chlorophenoxy)-piperidin-1-ylmethyl]-2,2,2-trifluoroethyl ester hydrochloride;
(4-Chlorophenyl)-carbamic acid (S)-1-[3-(4-cyanophenoxy)-piperidin-1-ylmethyl]-2,2,2-trifluoroethyl ester hydrochloride;
(4-Chlorophenyl)-carbamic acid (S)-1-[3-(3-cyanophenoxy)-piperidin-1-ylmethyl]-2,2,2-trifluoroethyl ester hydrochloride;
(4-Chlorophenyl)-carbamic acid (S)-1-[3-(4-fluorophenoxy)-piperidin-1-ylmethyl]-2,2,2-trifluoroethyl ester hydrochloride;
(4-Chlorophenyl)-carbamic acid (S)-1-[3-(3-methoxyphenoxy)-piperidin-1-ylmethyl]-2,2,2-trifluoroethyl ester hydrochloride;
(4-Chlorophenyl)-carbamic acid (S)-2,2,2-trifluoro-1-[3-(3-trifluoromethoxyphenoxyl)piperidin-1-ylmethyl]-ethyl ester hydrochloride;
(4-Chlorophenyl)-carbamic acid (S)-2,2,2-trifluoro-1-[3-(4-fluorophenoxymethyl)-piperidin-1-ylmethyl]-ethyl ester hydrochloride;
(4-Chlorophenyl)-carbamic acid (S)-1-[(S)-3-(4-chlorophenoxy)-piperidin-1-ylmethyl]-2,2,2-trifluoroethyl ester hydrochloride;
(4-Chlorophenyl)-carbamic acid (S)-1-[(R)-3-(4-chlorophenoxy)-piperidin-1-ylmethyl]-2,2,2-trifluoroethyl ester hydrochloride;
(4-Chlorophenyl)-carbamic acid (S)-2,2,2-trifluoro-1-(3′,4′,5′,6′-tetrahydro-2′H-[2,3]bipyridinyl-1′-ylmethyl)-ethyl ester hydrochloride;
(4-Chlorophenyl)-carbamic acid (S)-1-[3-(4-cyanopyridin-2-yloxy)-piperidin-1-ylmethyl]-2,2,2-trifluoroethyl ester hydrochloride;
(4-Chlorophenyl)-carbamic acid (S)-2,2,2-trifluoro-1-[3-(pyridin-3-ylmethoxy)-piperidin-1-ylmethyl]-ethyl ester hydrochloride;
(4-Chlorophenyl)-carbamic acid (S)-2,2,2-trifluoro-1-(6-trifluoromethyl-3′,4′,5′,6′-tetrahydro-2′H-[2,3]bipyridinyl-1′-ylmethyl)-ethyl ester hydrochloride;
(4-Chlorophenyl)-carbamic acid (S)-2,2,2-trifluoro-1-(6′-trifluoromethyl-3,4,5,6-tetrahydro-2H-[3,3]bipyridinyl-1-ylmethyl)-ethyl ester hydrochloride;
(5-Chloropyridin-2-yl)-carbamic acid (S)-2,2,2-trifluoro-1-[(R)-3-(3-trifluoromethylphenyl)-piperidin-1-ylmethyl]-ethyl ester hydrochloride;
(5-Chloropyridin-2-yl)-carbamic acid (S)-2,2,2-trifluoro-1-[3-(3-trifluoromethylphenyl)-piperidin-1-ylmethyl]-ethyl ester hydrochloride;
(4-Bromophenyl)-carbamic acid (S)-2,2,2-trifluoro-1-[3-(3-methoxyphenyl)-piperidin-1-ylmethyl]-ethyl ester hydrochloride;
(4-Fluorophenyl)-carbamic acid (S)-2,2,2-trifluoro-1-[3-(3-methoxyphenyl)-piperidin-1-ylmethyl]-ethyl ester hydrochloride;
(4-Chlorophenyl)-carbamic acid (S)-2,2,2-trifluoro-1-[3-(3-methanesulfonylphenyl)-piperidin-1-ylmethyl]-ethyl ester hydrochloride;
(4-Fluorophenyl)-carbamic acid (S)-2,2,2-trifluoro-1-[3-(3-methanesulfonylphenyl)-piperidin-1-ylmethyl]-ethyl ester hydrochloride;
(4-Chlorophenyl)-carbamic acid (S)-1-(3-benzylpyrrolidin-1-ylmethyl)-2,2,2-trifluoroethyl ester;
(3-Chloro-4-fluorophenyl)-carbamic acid (S)-2,2,2-trifluoro-1-[3-(3-trifluoromethylphenyl)-piperidin-1-ylmethyl]-ethyl ester;
(3,4-Dichloro-phenyl)-carbamic acid(S)-2,2,2-trifluoro-1-[3-(3-trifluoromethyl-phenyl)piperidin-1-ylmethyl]-ethyl ester;
(3,4-Difluorophenyl)-carbamic acid (S)-2,2,2-trifluoro-1-[3-(3-trifluoromethylphenyl)piperidin-1-ylmethyl]-ethyl ester; or
(6-Chloropyridin-3-yl)-carbamic acid (S)-2,2,2-trifluoro-1-[3-(3-trifluoromethylphenyl)-piperidin-1-ylmethyl]-ethyl ester.

16. A pharmaceutical composition, comprising a therapeutically effective amount of a compound of claim 1 and a pharmaceutically acceptable carrier.

17. A method for the treatment or prophylaxis of a respiratory disorder, comprising the step of administering a therapeutically effective amount of a compound according to claim 1 to a subject in need thereof.

18. The method of claim 17, wherein the respiratory disorder is selected from the group consisting of chronic obstructive pulmonary disorder (COPD), asthma, allergic rhinitis and bronchospasm.
